(19) United States
(12) Patent Application Publication
(10) Pub. No.: US 2026/0258105 A1
(43) Pub. Date: Sep. 3, 2026

(54) METHODS AND COMPOSITIONS FOR TREATING INFLAMMATORY AND AUTOIMMUNE CONDITIONS

(71) Applicants: THE UNIVERSITY OF CHICAGO, Chicago, IL (US); NIPPI, INC., Tokyo (JP)

(72) Inventors: Jeffrey A. HUBBELL, Chicago, IL (US); Tomonori UENO, Tokyo (JP); Kazunori MIZUNO, Tokyo (JP); Jun ISHIHARA, Chicago, IL (US); Ako ISHIHARA, Chicago, IL (US); Erica BUDINA, Chicago, IL (US)

(73) Assignees: THE UNIVERSITY OF CHICAGO, Chicago, IL (US); NIPPI, INC., Tokyo (JP)

(21) Appl. No.: 18/878,250

(22) PCT Filed: Jun. 23, 2023
(86) PCT No.: PCT/US2023/068949

§ 371 Date: Dec. 23, 2024

Related U.S. Application Data
(60) Provisional application No. 63/355,446, filed on Jun. 24, 2022.

(51) Int. Cl. C07K 14/54; A61K 9/00; A61K 38/00; A61K 45/06; A61P 19/02; A61P 37/06; C07K 14/765; G01N 33/68

(52) U.S. Cl. CPC C07K 14/54; A61K 9/0019; A61K 45/06; A61P 19/02; A61P 37/06; C07K 14/765; G01N 33/6863; A61K 38/00; C07K 2319/30; C07K 2319/31; G01N 2333/52

(57) ABSTRACT

The disclosure relates to the engineering of IL-35 cytokine polypeptides to achieve improved therapies for inflammatory diseases and conditions. Accordingly, aspects of the disclosure relate to a polypeptide comprising SEQ ID NO:2, wherein the polypeptide comprises one or more amino acid substitutions relative to SEQ ID NO:2, wherein the one or more amino acid substitutions comprise K23, R74, R195, R197, or combinations thereof. Further aspects relate to a polypeptide comprising SEQ ID NO:4, wherein the polypeptide comprises one or more amino acid substitutions relative to SEQ ID NO:4, wherein the one or more amino acid substitutions comprise C74. Further aspects relate to a polypeptide comprising SEQ ID NO:6, wherein the polypeptide comprises one or more amino acid substitutions relative to SEQ ID NO:6, wherein the one or more amino acid substitutions comprise C34.

This application claims the benefit of priority to U.S. Provisional Patent Application Ser. No. 63/355,446, filed Jun. 24, 2022, hereby incorporated by reference in its entirety.

### SEQUENCE LISTING

The instant application contains a Sequence Listing which has been submitted in XML format and is hereby incorporated by reference in its entirety. Said XML copy, created on Jun. 24, 2023, is named “ARCDP0778WO.xml” and is 122,913 bytes in size.

### BACKGROUND

### I. Field of the Invention

The invention generally relates to the field of medicine. More particularly, it concerns compositions and methods involving engineered anti-inflammatory agents for treating autoinflammatory and inflammatory conditions.

### II. Background

Multiple sclerosis (MS) is a potentially disabling autoimmune disease that affects millions globally. Autoreactive immune cells home to the central nervous system (CNS) and cause demyelination and consequently focal damage to white matter. Lymphocytes and macrophages that have infiltrated into the CNS cause axonal damage. Recent studies have shown that Th17 cells, activated in the secondary lymphoid organs (SLOs), migrate to the spinal cord and brain and play a crucial role in the disease development and severity of MS. Thus, inhibition of lymphocyte migration to the CNS and inducing an immune-suppressive microenvironment in the SLOs would provide an effective therapy for MS. FTY720 (fingolimod) and anti-integrin α4 antibody (natalizumab) are used in the clinic for treating MS, sequestering lymphocytes in the LNs and preventing them from reacting with autoantigens in target tissues. Experimental autoimmune encephalomyelitis (EAE) is a widely accepted murine model of MS, reflecting many features of disease progression and developmental mechanism, including lymphocyte migration to the CNS and demyelination.

Rheumatoid arthritis (RA) is an autoimmune disease that is currently controlled through treatment with inhibitors of inflammatory pathways. Pathological features of RA are synovitis and joint destruction, which cause severe pain and joint dysfunction. Although the causal antigen for RA has not been fully elucidated, collagen recognition by immune cells plays a key role. During progression of RA, autoantigen-specific T cells, especially Th17 cells, are activated and produce inflammatory cytokines including IL-17. Inflammatory cytokines, such as TNF-a and IL-6, in the joint induce activation of macrophages and neutrophils as mediators of the inflammatory response. These inflammatory cells infiltrate the joints and cause various inflammatory responses including activation of osteoclasts that destroy the bones in the joint. The current strategy for RA treatment is symptomatic, and considering that many inflammatory cytokines are involved RA progression, various biological therapeutics, such as antibodies or soluble receptors for TNF-a have been developed and approved for clinical use.

Various strategies for treatment of autoimmune disorders, such as MS and RA, have been explored involving the use of anti-inflammatory cytokines. Thus far, such strategies have not been translated to clinical use. There remains a need for compositions and methods for treatment of autoimmune disorders, including MS and RA, involving effective delivery of anti-inflammatory cytokines.

### SUMMARY OF INVENTION

The disclosure relates to the engineering of IL-35 cytokine polypeptides to achieve improved therapies for inflammatory diseases and conditions. Accordingly, the disclosure describes a polypeptide the amino acid sequence of SEQ ID NO:2 with one or more amino acid substitutions relative to SEQ ID NO:2, wherein the one or more amino acid substitutions comprise K23, R74, R195, R197, or combinations thereof. Substitution of K23, R74, R195, and/or R197, with reference to SEQ ID NO:2 may also be excluded in the polypeptides of the disclosure. Also disclosed is a polypeptide comprising the amino acid sequence of SEQ ID NO: 4 with one or more amino acid substitutions relative to SEQ ID NO:4, wherein the one or more amino acid substitutions comprise C74. Polypeptides may exclude a polypeptide of SEQ ID NO: 4 with a C74 substitution. Also provided is a comprising the amino acid sequence of SEQ ID NO:6 with one or more amino acid substitutions relative to SEQ ID NO:6, wherein the one or more amino acid substitutions comprise C34. Polypeptides may exclude a polypeptide of SEQ ID NO:6 with a C34 substitution. Also described is a polypeptide comprising the amino acid sequence of SEQ ID NO:90 with one or more amino acid substitutions relative to SEQ ID NO: 90, wherein the one or more amino acid substitutions comprise K2, R53, R174, R176, or combinations thereof. Substitution of K2, R53, R174, and/or R176, with reference to SEQ ID NO: 90 may also be excluded in the polypeptides of the disclosure. Also described is a polypeptide comprising the amino acid sequence of SEQ ID NO:1 with one or more amino acid substitutions relative to SEQ ID NO:1, wherein the one or more amino acid substitutions comprise K23, R74, R195, R197, C320, C497, or combinations thereof. Substitution of K23, R74, R195, R197, C320, and/or C497, with reference to SEQ ID NO: 1 may also be excluded in the polypeptides of the disclosure. Also described is a polypeptide comprising an EBI3 subunit of IL-35, wherein the EBI3 subunit comprises fewer than 200 amino acids and comprises the amino acid sequence of SEQ ID NO:92 or an amino acid sequence having at least 70% sequence identity to SEQ ID NO:92. Also described is a polypeptide comprising the amino acid sequence of SEQ ID NO:91 with one or more amino acid substitutions relative to SEQ ID NO:91, wherein the one or more amino acid substitutions comprise K2, R53, R174, R176, C299, C476, or combinations thereof. Substitution of K2, R53, R174, R176, C299, and/or C476, with reference to SEQ ID NO:91 may also be excluded in the polypeptides of the disclosure.

Also described is a chimeric or multimeric polypeptide comprising (i) the amino acid sequence of SEQ ID NO:2 with one or more amino acid substitutions relative to SEQ ID NO: 2, wherein the one or more amino acid substitutions relative to SEQ ID NO:2 comprise K23, R74, R195, R197, or combinations thereof; and (ii) the polypeptide of SEQ ID NO:4, a polypeptide having at least 70% sequence identity to SEQ ID NO:4; or a polypeptide comprising SEQ ID NO:4 with one or more substitutes relative to SEQ ID NO:4. Also described is a chimeric or multimeric polypeptide comprising: (i) the amino acid sequence of SEQ ID NO:90 with one or more amino acid substitutions relative to SEQ ID NO:90, wherein the one or more amino acid substitutions relative to SEQ ID NO:90 comprise K2, R53, R174, R176, or combinations thereof; and (ii) the polypeptide of SEQ ID NO:4, a polypeptide having at least 70% sequence identity to SEQ ID NO:4; or a polypeptide comprising SEQ ID NO:4 with one or more substitutes relative to SEQ ID NO:4. Also provided is a chimeric or multimeric polypeptide comprising: (i) an EBI3 subunit of IL-35, wherein the EBI3 subunit comprises less than 200 amino acids and comprises the amino acid sequence of SEQ ID NO:92 or an amino acid sequence having at least 70% sequence identity to SEQ ID NO:92 and (ii) the polypeptide of SEQ ID NO:4, a polypeptide having at least 70% sequence identity to SEQ ID NO: 4; or a polypeptide comprising SEQ ID NO:4 with one or more substitutes relative to SEQ ID NO: 4

Also provided are pharmaceutical compositions and host cells comprising polypeptides of the disclosure. The disclosure also provides for nucleic acids encoding the polypeptides of the disclosure, expression vectors comprising the nucleic acids, and host cells comprising the nucleic acids and/or expression vectors of the disclosure. The compositions may also comprise the nucleic acids, expression vectors, and/or host cells of the disclosure.

Methods relate to a method of making a cell comprising transferring nucleic acid(s) or expression vector(s) of the disclosure into a cell. Also provided is a method for making a polypeptide comprising transferring expression vector(s) of the disclosure into a cell and incubating the cell under conditions sufficient for expression of the polypeptide encoded on the expression vector. Also described is a method for treating an autoimmune or inflammatory condition in a subject comprising administering a polypeptide or pharmaceutical composition of the disclosure to the subject. Also provided a method for reducing inflammation in a subject comprising administering a polypeptide or pharmaceutical composition of the disclosure to the subject. Methods also include methods for delivering cytokine therapy to lymph nodes and/or for increasing tolerogenic dendritic cells in the lymph nodes in a subject comprising administering a polypeptide or pharmaceutical composition of the disclosure to the subject.

The EBI3 polypeptide may have or have at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:92. The EBI3 subunit may comprise one or more amino acid substitutions and wherein the one or more amino acid substitutions comprise R33, R154, R156, or combinations thereof, relative to SEQ ID NO:92. Substitution of R33, R154, and/or R156, with reference to SEQ ID NO:92 may also be excluded in the polypeptides of the disclosure. The EBI3 subunit may consist of 189 amino acids. The EBI3 subunit may comprise, comprise at most, comprise at least, or consist of 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, or 209 amino acids, or any derivable range therein. The polypeptide may comprise substitutions of C6, C39, C49, or combinations thereof, relative to SEQ ID NO:92. Substitution of C6, C39, and/or C49, with reference to SEQ ID NO:92 may also be excluded in the polypeptides of the disclosure. The substitutions may be with a serine. The polypeptide may comprise 1, 2, or 3 of the substitutions C6, C39, and C49. The EBI3 subunit may be amino-proximal to the polypeptide with the amino acid sequence of SEQ ID NO:4, the polypeptide with an amino acid sequence having at least 70% sequence identity to SEQ ID NO:4, or the polypeptide comprising the substituted amino acid sequence of SEQ ID NO:4. The EBI3 subunit may be carboxy-proximal to the polypeptide with the amino acid sequence of SEQ ID NO:4, the polypeptide with an amino acid sequence having at least 70% sequence identity to SEQ ID NO: 4, or the polypeptide comprising the substituted amino acid sequence of SEQ ID NO:4. A first region is carboxy-proximal to a second region when the first region is attached to the carboxy terminus of the second region. There may be further intervening amino acid residues between the first and second regions. Thus, the regions need not be immediately adjacent, unless specifically specified as not having intervening amino acid residues. The term “amino-proximal” is similarly defined in that a first region is amino-proximal to a second region when the first region is attached to the amino terminus of the second region. Similarly, there may be further intervening amino acid residues between the first and second regions unless stated otherwise.

The polypeptide and/or polypeptide comprising the substituted amino acid sequence of SEQ ID NO:2 may comprise an amino acid sequence having at least 70% sequence identity to SEQ ID NO:2. The polypeptide may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to SEQ ID NO:2. The polypeptide may comprise substitutions of one or more of C36, C47, C80, and/or C90, relative to SEQ ID NO:2. Substitution of C36, C47, C80, and C90, with reference to SEQ ID NO:2 may also be excluded in the polypeptides of the disclosure. The substitutions may be a substitution with a serine. The polypeptide may comprise 1, 2, 3, or 4 of substitutions at C36, C47, C80, and C90.

The polypeptide and/or polypeptide comprising the substituted amino acid sequence of SEQ ID NO:90 may comprise an amino acid sequence having at least 70% sequence identity to SEQ ID NO:90. The polypeptide may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to SEQ ID NO:90. The polypeptide may comprise substitutions of one or more of C15, C26, C59, and C69, relative to SEQ ID NO:90. Substitution of C15, C26, C59, and/or C69, with reference to SEQ ID NO:90 may also be excluded in the polypeptides of the disclosure. The substitutions may be a substitution with a serine. The polypeptide may comprise 1, 2, 3, or 4 of substitutions at C15, C26, C59, and C69.

The polypeptide may comprise an amino acid sequence that has at least 70% sequence identity to SEQ ID NO:4. The polypeptide may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to SEQ ID NO:4. The polypeptide comprising one or more amino acid substitutions relative to SEQ ID NO:4 may comprise a substitution of C74. Substitution of C74, with reference to SEQ ID NO:4 may also be excluded in the polypeptides of the disclosure.

The polypeptide may comprise an amino acid sequence that has at least 70% sequence identity to SEQ ID NO:6. The polypeptide may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to SEQ ID NO:6.

The polypeptide may comprise an amino acid sequence that has at least 70% sequence identity to SEQ ID NO:1. The polypeptide may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to SEQ ID NO:1. The one or more amino acid substitutions may comprise K23, C320, and/or C497, relative to SEQ ID NO:1.

The polypeptide may comprise an amino acid sequence that has at least 70% sequence identity to SEQ ID NO:91. The polypeptide may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to SEQ ID NO:91. The one or more amino acid substitutions may comprise K2, C299, and/or C476, relative to SEQ ID NO:91. The polypeptide may comprise substitutions of C15, C26, C59, C69, or combinations thereof, relative to SEQ ID NO:91. Substitution of C15, C26, C59, and/or C69, with reference to SEQ ID NO: 91 may also be excluded in the polypeptides of the disclosure. The substitutions may be a substitution with a serine. The polypeptide may comprise 1, 2, 3, or 4 of substitutions at C15, C26, C59, and C69.

The polypeptide may further comprise or may exclude an ECM-affinity peptide, serum protein, albumin binding protein, and/or Fc peptide. ECM-affinity peptides are described throughout the disclosure. The ECM-affinity peptide may comprise or exclude an amino acid sequence of one of SEQ ID NOs: 9-27 or 88 or an amino acid sequence having at least 70% sequence identity to one of SEQ ID NOs: 9-27 or 88. The ECM-affinity peptide may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to one of SEQ ID NOs: 9-27 or 88. The Fc peptide may comprise or exclude an amino acid sequence of SEQ ID NO:86 or 87 or an amino acid sequence having at least 70% sequence identity to SEQ ID NO: 86 or 87. The Fc peptide may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to SEQ ID NO:86 or 87. The ABP may comprise or exclude the amino acid sequence of SEQ ID NO:93 or an amino acid sequence having at least 70% sequence identity to SEQ ID NO:93. The ABP may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to SEQ ID NO:93. The ECM-affinity peptide, serum protein, ABP, and/or Fc peptide may be amino-proximal to the polypeptide comprising the substituted amino acid sequence of one of SEQ ID NO:1, 2, 4, 6, 90, 91 or the EBI3 subunit. The ECM-affinity peptide, serum protein, and/or Fc peptide may be carboxy-proximal to the polypeptide comprising the substituted amino acid sequence of one of SEQ ID NO:1, 2, 4, 6, 90, 91 or the EBI3 subunit. The ECM-affinity peptide, serum protein, ABP, and/or Fc peptide may be amino-proximal to (i) and/or (ii), as defined herein.

The serum protein may comprise a polypeptide having the amino acid sequence of SEQ ID NO:6 or a polypeptide with at least 70% sequence identity to a polypeptide having the amino acid sequence of SEQ ID NO:6. The serum protein may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to SEQ ID NO:6. The polypeptide may comprise one or more amino acid substitutions relative to SEQ ID NO:6, wherein the one or more amino acid substitutions comprise C34. The substitution at C34 may be with an alanine or serine amino acid residue.

The one or more amino acid substitutions relative to SEQ ID NO:2 may comprise K23 and the one or more amino acid substitutions relative to SEQ ID NO:4 may comprise C74. The K23 and/or C74 substitutions may be with an alanine or serine amino acid residues. The K23 and C74 substitutions may comprise a K23A and/or C74S substitution(s).

The polypeptide comprising the substituted amino acid sequence of SEQ ID NO:2 may be amino-proximal to the polypeptide with the amino acid sequence of SEQ ID NO:4, the polypeptide with an amino acid sequence having at least 70% sequence identity to SEQ ID NO: 4, or the polypeptide comprising the substituted amino acid sequence of SEQ ID NO:4. The polypeptide comprising the substituted amino acid sequence of SEQ ID NO:2 may be carboxy-proximal to the polypeptide with the amino acid sequence of SEQ ID NO:4, the polypeptide with an amino acid sequence having at least 70% sequence identity to SEQ ID NO: 4, or the polypeptide comprising the substituted amino acid sequence of SEQ ID NO:4.

The polypeptide comprising the substituted amino acid sequence of SEQ ID NO:90 may be amino-proximal to the polypeptide with the amino acid sequence of SEQ ID NO:4, the polypeptide with an amino acid sequence having at least 70% sequence identity to SEQ ID NO: 4, or the polypeptide comprising the substituted amino acid sequence of SEQ ID NO:4. The polypeptide comprising the substituted amino acid sequence of SEQ ID NO:90 may be carboxy-proximal to the polypeptide with the amino acid sequence of SEQ ID NO:4, the polypeptide with an amino acid sequence having at least 70% sequence identity to SEQ ID NO: 4, or the polypeptide comprising the substituted amino acid sequence of SEQ ID NO:4.

The one or more amino acid substitutions relative to SEQ ID NO:90 may comprise or consist of K2. The one or more amino acid substitutions relative to SEQ ID NO:4 may comprise or consist of C74. The polypeptide may comprise or consist of substitutions at K2 and C74 of SEQ ID NOs: 90 and 4, respectively. The substitutions may comprise a K2A and C74S.

Linkers, such as amino acid or peptidomimetic sequences may be inserted in the polypeptides of the disclosure. Linkers may have one or more properties that include a flexible conformation, an inability to form an ordered secondary structure or a hydrophobic or charged character which could promote or interact with either domain. Examples of amino acids typically found in flexible protein regions may include Gly, Asn and Ser. For example, a suitable peptide linker may be GGGSGGGS (SEQ ID NO:7) or (GGGS) n (SEQ ID NO:8), wherein n=1, 2, 3, 4, 5, 6, 7, 8, 9, or 10 (or any range derivable therein). Other near neutral amino acids, such as Thr and Ala, may also be used in the linker sequence. The length of the linker sequence may vary without significantly affecting the function or activity of the fusion protein (see, e.g., U.S. Pat. No. 6,087,329). A peptide and an antibody heavy or light chain may be joined by a peptide sequence having from about 1 to 25 amino acid residues. Examples of linkers may also include chemical moieties and conjugating agents, such as sulfosuccinimidyl derivatives (sulfo-SMCC, sulfo-SMPB), disuccinimidyl suberate (DSS), disuccinimidyl glutarate (DSG) and disuccinimidyl tartrate (DST). Examples of linkers further comprise a linear carbon chain, such as C_{N} (where N=1-100 carbon atoms, e.g., N=2, 3, 4, 5, 6, 7, 8, 9, or 10). The linker can be a dipeptide linker, such as a valine-citrulline (val-cit), a phenylalanine-lysine (phe-lys) or linker, maleimidocapronic-valine-citruline-p-aminobenzyloxycarbonyl (vc) linker. The linker may be sulfosuccinimidyl-4-[N-maleimidomethyl]cyclohexane-1-carboxylate (smcc). Sulfo-smcc conjugation occurs via a maleimide group which reacts with sulfhydryls (thiols, —SH), while its sulfo-NHS ester is reactive toward primary amines (as found in lysine and the protein or peptide N-terminus).

Further, the linker may be maleimidocaproyl (mc). One or more of these linkers may be specifically excluded from an aspect.

The chimeric polypeptide may comprise a linker between (i) and (ii), as defined herein. There may also be linkers between (i) and (ii) and a serum protein, Fc peptide, ABP, HSA, and/or ECM-affinity peptide. The linker may comprise or consist of the amino acid sequence of one of SEQ ID NOs: 3, 5, 7, 8, or 75-85. The linker may comprise glycine and serine residues. The linker may comprise a (G3S) n linker, wherein n is an integer from 1 to 10. The linker may comprise or consist of the amino acid sequence of SEQ ID NO:3. The linker may comprise or consist of the amino acid sequence of SEQ ID NO:5. The linker may comprise or consist of the amino acid sequence of SEQ ID NO:75. Linkers between, amino-proximal to, carboxy-proximal to, or flanking one or more of (i), (ii), a serum protein, Fc peptide, ABP, and/or ECM-affinity peptide may be excluded in the polypeptides of the disclosure.

The polypeptide may comprise or further comprise a serum albumin (SA) polypeptide. The SA polypeptide may be further defined as a human SA polypeptide. The SA polypeptide may comprise a polypeptide having the amino acid sequence of SEQ ID NO:6 or a polypeptide with at least 70% sequence identity to a polypeptide with the amino acid sequence of SEQ ID NO:6. The SA polypeptide may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to SEQ ID NO:6. The polypeptide may comprise one or more amino acid substitutions relative to SEQ ID NO:6, wherein the one or more amino acid substitutions comprise C34. The substitution at C34 may be a substitution of a cysteine for any other amino acid. The substitution at C34 may be with an alanine or serine amino acid residue. The SA polypeptide may be carboxy-proximal (i) and/or (ii), as defined herein. The SA polypeptide may be amino-proximal to (i) and/or (ii), as defined herein . . .

The substitution may be a substitution with a nonpolar amino acid. The substitution at position K23, R74, R195, R197 relative to SEQ ID NO:2, position C74 relative to SEQ ID NO: 4, position C34 relative to SEQ ID NO:6, or positions K23, R74, R195, R197, C320, C497 relative to SEQ ID NO:1 may be a substitution with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine amino acid residue. The substitution may be a substitution with an alanine, leucine, isoleucine, proline, tryptophan, valine, phenylalanine, or methionine. The substitutions described herein may exclude or include a substitution with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine amino acid residue. The substitution may be a substitution with an alanine, leucine, isoleucine, proline, tryptophan, valine, phenylalanine, or methionine. The substitution at position K23, R74, R195, R197 relative to SEQ ID NO:2, position C74 relative to SEQ ID NO:4, position C34 relative to SEQ ID NO: 6, or positions K23, R74, R195, R197, C320, C497 relative to SEQ ID NO: 1 may be a substitution with an alanine, leucine, isoleucine, proline, tryptophan, valine, phenylalanine, or methionine. The substitution may be with an alanine. The substitution may be with a serine or glycine. Substitutions with lysine or arginine may be excluded in the polypeptides of the disclosure. Substitutions with any other amino acid may also be excluded in any polypeptide of the disclosure.

Polypeptides of the disclosure may comprise a signal peptide. The signal peptide may comprise SEQ ID NO:89. Polypeptides of the disclosure may comprise or consist of a polypeptide comprising the amino acid sequence of one or more of SEQ ID NOS: 1-93, or an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to one or more of SEQ ID NOS: 1-93. Any of SEQ ID NOS: 1-93 may be excluded in the polypeptides of the disclosure.

The inflammation that is treated by the polypeptides and compositions of the disclosure, may be inflammation associated with an autoimmune or inflammatory condition. The autoimmune or inflammatory condition may comprise or exclude cytokine release syndrome, cytokine storm, arthritis, inflammatory bowel disease, scleroderma, inflammatory bowel disease, idiopathic pulmonary fibrosis, multiple sclerosis, type 1 diabetes, Crohn's disease, psoriasis, acute inflammation, chronic inflammation, neuroinflammation, acute respiratory distress syndrome, rheumatoid arthritis, fibrosis, infection, allergy, inflammatory therapy-related adverse events, ischemic stroke, pulmonary hypertension, ischemia, cardiac stroke, brain stroke, atherosclerosis, wound healing, peripheral artery disease, cerebral ischemia-reperfusion (I/R) injury, myocardial infarction, and related inflammatory illness. The inflammation treated may be inflammation associated with a viral infection, such as the flu, coronavirus, or SARS-COV-2 infection. The inflammation treated may be inflammation associated with administration of an immunotherapy, such as administration of checkpoint inhibitor therapy, administration of CAR-T cells, or administration of engineered TCR-T cells. The method may be for treating cytokine release syndrome or cytokine storm. The autoimmune or inflammatory condition may comprise multiple sclerosis. The autoimmune or inflammatory condition may comprise rheumatoid arthritis.

The composition or polypeptide may be administered by any route of administration, which are described throughout the disclosure. The composition or polypeptide may be administered systemically. The composition or polypeptide may be administered by intravenous injection. The composition may be administered locally. The composition may be administered to or adjacent to a site of inflammation.

The subject may be one that has been previously treated with an anti-inflammatory agent, anti-inflammatory therapy, or autoimmune therapy. The subject may be one that has been determined to be non-responsive to the previous treatment. The subject may be one that has not been treated previously for the inflammatory or autoimmune disease. The method may further comprise administration of an additional inflammatory or autoimmune therapy. The additional inflammatory or autoimmune therapy may comprise or exclude an anti-cytokine agent or a steroid. The additional inflammatory or autoimmune therapy may comprise or exclude one or more of steroids, corticosteroids, anti-TNF-alpha therapy, anti-integrin therapy, infliximab, mesalamine, and vedolizumab, fingolimod, interferon-β, dimethyl fumarate, teriflunomide, integrin α4β1, an anti-αLβ2 antibody, an anti-IL-6R agent, an anti-IL-6 agent, and a Janus kinase inhibitor (e.g., tofacitinib, baricitinib, upadacitinib). The steroid may comprise or exclude dexamethasone, methylprednisolone, and/or prednisolone. The polypeptide or composition may be one that decreases a number of Th17 cells in the subject. The polypeptide or composition may be one that inhibits a function of Th17 cells in the subject. The polypeptide or composition may be administered to the subject via a pre-filled syringe.

The polypeptide or composition may be administered at a dose of between 0.1 mg/kg and 50 mg/kg. The polypeptide or composition may be administered at a dose of, of at least, or of at most 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 μg, ng, μg, mg, g, pg/kg, ng/kg, μg/kg, mg/kg, or g/kg (or any derivable range therein), wherein “/kg” refers to a dose administered per kg of the subject.

The method may comprise further comprising detecting the anti-inflammatory cytokine in a lymph node of the subject. The method may comprise obtaining a lymph sample from the subject. Detecting may comprise detecting the presence of the polypeptide of the disclosure in the lymph sample.

The terms “protein”, “polypeptide” and “peptide” are used interchangeably herein when referring to a gene product comprising a polymer of amino acids.

The terms “subject,” “mammal,” and “patient” are used interchangeably. The subject may be a mammal. The subject may be a human. The subject may be a mouse, rat, rabbit, dog, donkey, or a laboratory test animal such as fruit fly, zebrafish, etc.

It is contemplated that the methods and compositions include exclusion of any of the aspects described herein.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments or aspects described in the context of the term “comprising” may also be implemented in the context of the term “consisting of” or “consisting essentially of.”

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments or aspects discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments or aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Secretion of recombinant proteins expressed by CHO cells. Four sites (K22A, R194A, R73A, and R196A) were selected that were especially effective.

FIG. 2. Study of combinations of mutation sites

FIG. 3. Mutant strains cultured under conditions of increased antioxidant stress tolerance resulted in significantly reduced aggregate formation and increased secretion of IL-35-HSA.

FIG. 4. Optimization of linker sequence. We have made varions IL-35-HSA molecules with different linker domains between IL-35 and HSA. Linker ID: 1-12 are detailed in table 6. After protein production, we performed westernblotting to assess the IL35-HSA protein existence in the culture media.

FIG. 5. Expression vectors of the mutant, in which four cysteines in EBI3 were replaced with serine, were produced by CHO cells. The culture media were analyzed by western blotting to test IL-35 and image analysis of the obtained signal intensities was performed with ImageJ software to evaluate the amount of secreted proteins. The relative amount of secreted protein was calculated with the IL-35-HSA/WT as 1. Replacing cysteine with serine at four locations in EBI3 slightly increased IL-35-HSA secretion.

FIG. 6. IL-35-HSA was observed to multimerize in the culture medium and during the purification process. In order to monomerize the multimerized IL-35-HSA, additives were investigated. Culture supernatants of CHO cells stably expressing IL-35-HSA were concentrated by ultrafiltration membrane and replaced with 30 mM phosphate buffer (pH 7) and 50 mM Tris buffer (pH 9), respectively. After equal amounts were dispensed into each tube, each additive was added. The additives used were ascorbic acid (final 1 mM), methionine (final 1 mM), glutathione (final 1 mM), and cysteine (final 1, 10, 50 mM), respectively. The samples were then incubated at 37° C. for 15 minutes. Each secretion profile of IL-35-HSA were then analyzed by western blotting.

FIG. 7. HEK239-derived IL-35-HSA can become monomer after incubation with 5 mM DTT for 15 mins, analyzed by SDS-PAGE.

FIG. 8. The inventors made N-terminus deletion variants of IL-35-HSA and analyzed by SDS-PAGE (10 amino acids deletion: delta10 and 20 amino acids deletion: delta20). Culture supernatants of His6-tagged IL-35-HSA transfected HEK293 cells was purified using His-tag purification Ni2+ column and then further purified with size exclusion column in PBS. Non-Reducing condition of SDS-PAGE shows that 20 amino acid deletion, not 10 amino acid deletion solve the ultra-large aggregation. (FIG. 8)

FIG. 9. Mice were injected 40 μg of IL-35-HSA subcutaneously in the back skin. 4 hours and 8 days after injection, the inventors collected lymph nodes. After hominization of lymph node, they tested the existence of human IL-35 using a ELISA kit to detect human EBI3 protein. Signal of 450 nm shows that IL-35-HSA is detectable and existing in lymph nodes for long-term.

FIG. 10A-10D. (A) 10 μg IL-35-HSA protein was injected twice subcutaneously on day 0 and day 7 to C57B6 mice. On day 9, 2.5 μg TNF-alpha (TNFa) was injected i.p. Two hours after TNFa injection, the inventors collected blood plasma from mice. ELISA was performed to detect IL-6 in the plasma. (B-D) 10 μg IL-35 proteins were injected twice subcutaneously on day 0 and day 7 to C57B6 mice. On day 7, the inventors collected lymph nodes from mice and analyzed immune cell populations using a flow cytometry.

FIG. 11A-11C: Evaluation of SA IL-35 as a prophylactic treatment in collagen antibody induced arthritis. (A) Collagen antibody induced arthritis experiment timeline. (B) Arthritis clinical score results from day 3 to 12. (C) Arthritis clinical scores at endpoint. Mutated SA IL-35 treatment prevents the onset of severe disease.

FIG. 12A-12F. (A) T-cell immunophenotyping in the popliteal lymph nodes. Mutated SA IL-35 treatment elevates PD-1 expression in CD4⁺ and CD8⁺ T cells. (B) T-cell immunophenotyping in the spleen. Mutated SA IL-35 treatment elevates PD-1 expression in CD4⁺ T cells. (C) Myeloid cell immunophenotyping in the popliteal lymph nodes. Mutated SA IL-35 treatment reduces the expression of CD40 and CD86 on CD11b⁺ myeloid cells. Treatment also reduces the percentage of CD11b⁺F4/80⁺macrophages in the lymph nodes as well as the expression of CD86 on macrophages, while increasing the M2 to M1 macrophage ratio. (D) Myeloid cell immunophenotyping in the spleen. Mutated SA IL-35 treatment reduces the percentage of CD11b⁺myeloid cells in the spleen and the expression of CD86 on CD11b⁺ cells. Treatment also reduces the percentage of CD11b⁺F4/80⁺macrophages while increasing the percentage of Arg1⁺M2 macrophages. (E) Dendritic cell immunophenotyping in the lymph nodes. Mutated SA IL-35 treatment reduces the percentage of CD11c⁺dendritic cells and the expression of CD86 and CD40 on CD11c⁺ dendritic cells. Treatment also reduces the percentage of CD11c⁺CD11b⁺ dendritic cells and the expression of CD86, CD40 and MHC class II on these cells. (F) Myeloid-derived suppressor cell immunophenotyping in the lymph nodes. Mutated SA IL-35 treatment reduced the expression of CD86 and CD40 on CD11b⁺Ly6C^{hi} Ly6G⁻MDSCs.

### DETAILED DESCRIPTION

### I. ECM-Affinity Peptides

Collagen is an extracellular matrix (ECM)-protein that regulates a variety of cellular biological functions, such as proliferation, differentiation, and adhesion in both normal and tumor tissue (Ricard-Blum, Cold Spring Harb Perspect Biol 3: a004978, 2011). Collagen is the most abundant protein in the mammalian body and exists in almost all tissues in one or more of 28 isoforms (Ricard-Blum, Cold Spring Harb Perspect Biol 3: a004978, 2011). The blood vessel sub-endothelial space is rich in collagen. Because of its insolubility under physiological conditions, collagen barely exists within the blood (Dubois et al., Blood 107:3902-06, 2006; Bergmeier and Hynes, Cold Spring Harb Perspect Biol 4: a005132, 2012). Tumor vasculature is reported to be permeable due to an abnormal structure (Nagy et al., British journal of cancer 100:865, 2009). Thus, with its leaky vasculature, collagen is exposed in the tumor (Liang et al., Journal of controlled release 209:101-109, 2015; Liang et al., Sci Rep 6:18205, 2016; Yasunaga et al., Bioconjugate Chemistry 22:1776-83, 2011; Xu et al. The Journal of cell biology 154:1069-80, 2001; Swartz and Lund, Nat Rev Cancer 12:210-19). Also, tumor tissue contains increased amounts of collagen compared to normal tissues (Zhou et al. J Cancer 8:1466-76, 2017; Provenzano et al. BMC Med 6:11, 2008).

von Willebrand factor (vWF) is a blood coagulation factor and binds to both type I and type III collagen, and the adhesion receptor GPIb on blood platelets (Lenting et al., Journal of thrombosis and haemostasis: JTH 10:2428-37, 2012; Shahidi Advances in experimental medicine and biology 906:285-306, 2017). When injured, collagen beneath endothelial cells is exposed to blood plasma, and vWF-collagen binding initiates the thrombosis cascade (Shahidi Advances in experimental medicine and biology 906:285-306, 2017; Wu et al. Blood 99:3623-28, 2002). The vWF A domain has the highest affinity against collagen among reported non-bacterial origin proteins/peptides (Addi et al., Tissue Engineering Part B: Reviews, 2016). Particularly within the A domain, the A3 domain of vWF has been reported as a collagen binding domain (CBD) (Ribba et al. Thrombosis and Haemostasis 86:848-54, 2001). As described above, the inventors contemplated that a fusion protein with the vWF A3 CBD may achieve targeted cytokine immunotherapy even when injected systemically due to exposure of collagen via the leaky tumor vasculature.

The ECM-affinity peptide may comprise a collagen binding domain from decorin. The ECM-affinity peptide may comprise a decorin peptide such as LRELHLNNNC (SEQ ID NO: 9), which is derived from bovine or LRELHLDNNC (SEQ ID NO:10), which is derived from human.

The ECM-peptide may comprise a peptide fragment from human decorin, which is represented by the following amino acid sequence:

(SEQ ID NO: 11)
CGPFQQRGLFDFMLEDEASGIGPEVPDDRDFEPSLGPVCPFRCQCHLRV
VQCSDLGLDKVPKDLPPDTTLLDLQNNKITEIKDGDFKNLKNLHALILV
NNKISKVSPGAFTPLVKLERLYLSKNQLKELPEKMPKTLQELRAHENEI
TKVRKVTFNGLNQMIVIELGTNPLKSSGIENGAFQGMKKLSYIRIADTN
ITSIPQGLPPSLTELHLDGNKISRVDAASLKGLNNLAKLGLSFNSISAV
DNGSLANTPHLRELHLDNNKLTRVPGGLAEHKYIQVVYLHNNNISVVGS
SDFCPPGHNTKKASYSGVSLFSNPVQYWEIQPSTFRCVYVRSAIQLGNY
K.

The ECM-peptide may comprise a peptide fragment from vWF. The ECM-peptide may comprise vWF Al derived from human sequence, residues 1237-1458 (474-695 of mature VWF) or a fragment thereof, which is represented by the amino acid sequence

(SEQ ID NO: 12)
CQEPGGLVVPPTDAPVSPTTLYVEDISEPPLHDFYCSRLLDLVFLLDGS
SRLSEAEFEVLKAFVVDMMERLRISQKWVRVAVVEYHDGSHAYIGLKDR
KRPSELRRIASQVKYAGSQVASTSEVLKYTLFQIFSKIDRPEASRITLL
LMASQEPQRMSRNFVRYVQGLKKKKVIVIPVGIGPHANLKQIRLIEKQA
PENKAFVLSSVDELEQQRDEIVSYLC.

The ECM-peptide may comprise all or a fragment of vWF A3, which is represented by the following amino acid sequences:

(SEQ ID NO: 13)
CSQPLDVILLLDGSSSFPASYFDEMKSFAKAFISKANIGPRLTQVSVLQ
YGSITTIDVPWNVVPEKAHLLSLVDVMQREGGPSQIGDALGFAVRYLTS
EMHGARPGASKAVVILVTDVSVDSVDAAADAARSNRVTVFPIGIGDRYD
AAQLRILAGPAGDSNVVKLQRIEDLPTMVTLGNSFLHKLCSGFVRICTG
and
(SEQ ID NO: 14)
CSQPLDVILLLDGSSSFPASYFDEMKSFAKAFISKANIGPRLTQVSVLQ
YGSITTIDVPWNVVPEKAHLLSLVDVMQREGGPSQIGDALGFAVRYLTS
EMHGARPGASKAVVILVTDVSVDSVDAAADAARSNRVTVFPIGIGDRYD
AAQLRILAGPAGDSNVVKLQRIEDLPTMVTLGNSFLHKLCSGFVRI.

The ECM-peptide may comprise all or a fragment of vWF A3, which is represented by the amino acid following sequences:

(SEQ ID NO: 15)
CSQPLDVVLLLDGSSSLPESSFDKMKSFAKAFISKANIGPHLTQV
SVIQYGSINTIDVPWNVVQEKAHLQSLVDLMQQEGGPSQIGDALA
FAVRYVTSQIHGARPGASKAVVIIIMDTSLDPVDTAADAARSNRV
AVFPVGVGDRYDEAQLRILAGPGASSNVVKLQQVEDLSTMATLGN
SFFHKLCSGFSGV.

The ECM-affinity peptide may be a peptide from von Willebrand factor (vWF). The sequence of human VWF comprises the following:

(SEQ ID NO: 16)
MIPARFAGVLLALALILPGTLCAEGTRGRSSTARCSLFGSDFVNT
FDGSMYSFAGYCSYLLAGGCQKRSFSIIGDFQNGKRVSLSVYLGE
FFDIHLFVNGTVTQGDQRVSMPYASKGLYLETEAGYYKLSGEAYG
FVARIDGSGNFQVLLSDRYFNKTCGLCGNFNIFAEDDFMTQEGTL
TSDPYDFANSWALSSGEQWCERASPPSSSCNISSGEMQKGLWEQC
QLLKSTSVFARCHPLVDPEPFVALCEKTLCECAGGLECACPALLE
YARTCAQEGMVLYGWTDHSACSPVCPAGMEYRQCVSPCARTCQSL
HINEMCQERCVDGCSCPEGQLLDEGLCVESTECPCVHSGKRYPPG
TSLSRDCNTCICRNSQWICSNEECPGECLVTGQSHFKSFDNRYFT
FSGICQYLLARDCQDHSFSIVIETVQCADDRDAVCTRSVTVRLPG
LHNSLVKLKHGAGVAMDGQDVQLPLLKGDLRIQHTVTASVRLSYG
EDLQMDWDGRGRLLVKLSPVYAGKTCGLCGNYNGNQGDDFLTPSG
LAEPRVEDFGNAWKLHGDCQDLQKQHSDPCALNPRMTRFSEEACA
VLTSPTFEACHRAVSPLPYLRNCRYDVCSCSDGRECLCGALASYA
AACAGRGVRVAWREPGRCELNCPKGQVYLQCGTPCNLTCRSLSYP
DEECNEACLEGCFCPPGLYMDERGDCVPKAQCPCYYDGEIFQPED
IFSDHHTMCYCEDGFMHCTMSGVPGSLLPDAVLSSPLSHRSKRSL
SCRPPMVKLVCPADNLRAEGLECTKTCQNYDLECMSMGCVSGCLC
PPGMVRHENRCVALERCPCFHQGKEYAPGETVKIGCNTCVCRDRK
WNCTDHVCDATCSTIGMAHYLTFDGLKYLFPGECQYVLVQDYCGS
NPGTFRILVGNKGCSHPSVKCKKRVTILVEGGEIELFDGEVNVKR
PMKDETHFEVVESGRYIILLLGKALSVVWDRHLSISVVLKQTYQE
KVCGLCGNFDGIQNNDLTSSNLQVEEDPVDFGNSWKVSSQCADTR
KVPLDSSPATCHNNIMKQTMVDSSCRILTSDVFQDCNKLVDPEPY
LDVCIYDTCSCESIGDCACFCDTIAAYAHVCAQHGKVVTWRTATL
CPQSCEERNLRENGYECEWRYNSCAPACQVTCQHPEPLACPVQCV
EGCHAHCPPGKILDELLQTCVDPEDCPVCEVAGRRFASGKKVTLN
PSDPEHCQICHCDVVNLTCEACQEPGGLVVPPTDAPVSPTTLYVE
DISEPPLHDFYCSRLLDLVFLLDGSSRLSEAEFEVLKAFVVDMME
RLRISQKWVRVAVVEYHDGSHAYIGLKDRKRPSELRRIASQVKYA
GSQVASTSEVLKYTLFQIFSKIDRPEASRITLLLMASQEPQRMSR
NFVRYVQGLKKKKVIVIPVGIGPHANLKQIRLIEKQAPENKAFVL
SSVDELEQQRDEIVSYLCDLAPEAPPPTLPPDMAQVTVGPGLLGV
STLGPKRNSMVLDVAFVLEGSDKIGEADFNRSKEFMEEVIQRMDV
GQDSIHVTVLQYSYMVTVEYPFSEAQSKGDILQRVREIRYQGGNR
TNTGLALRYLSDHSFLVSQGDREQAPNLVYMVTGNPASDEIKRLP
GDIQVVPIGVGPNANVQELERIGWPNAPILIQDFETLPREAPDLV
LQRCCSGEGLQIPTLSPAPDCSQPLDVILLLDGSSSFPASYFDEM
KSFAKAFISKANIGPRLTQVSVLQYGSITTIDVPWNVVPEKAHLL
SLVDVMQREGGPSQIGDALGFAVRYLTSEMHGARPGASKAVVILV
TDVSVDSVDAAADAARSNRVTVFPIGIGDRYDAAQLRILAGPAGD
SNVVKLQRIEDLPTMVTLGNSFLHKLCSGFVRICMDEDGNEKRPG
DVWTLPDQCHTVTCQPDGQTLLKSHRVNCDRGLRPSCPNSQSPVK
VEETCGCRWTCPCVCTGSSTRHIVTFDGQNFKLTGSCSYVLFQNK
EQDLEVILHNGACSPGARQGCMKSIEVKHSALSVELHSDMEVTVN
GRLVSVPYVGGNMEVNVYGAIMHEVRFNHLGHIFTFTPQNNEFQL
QLSPKTFASKTYGLCGICDENGANDFMLRDGTVTTDWKTLVQEWT
VQRPGQTCQPILEEQCLVPDSSHCQVLLLPLFAECHKVLAPATFY
AICQQDSCHQEQVCEVIASYAHLCRTNGVCVDWRTPDFCAMSCPP
SLVYNHCEHGCPRHCDGNVSSCGDHPSEGCFCPPDKVMLEGSCVP
EEACTQCIGEDGVQHQFLEAWVPDHQPCQICTCLSGRKVNCTTQP
CPTAKAPTCGLCEVARLRQNADQCCPEYECVCDPVSCDLPPVPHC
ERGLQPTLTNPGECRPNFTCACRKEECKRVSPPSCPPHRLPTLRK
TQCCDEYECACNCVNSTVSCPLGYLASTATNDCGCTTTTCLPDKV
CVHRSTIYPVGQFWEEGCDVCTCTDMEDAVMGLRVAQCSQKPCED
SCRSGFTYVLHEGECCGRCLPSACEVVTGSPRGDSQSSWKSVGSQ
WASPENPCLINECVRVKEEVFIQQRNVSCPQLEVPVCPSGFQLSC
KTSACCPSCRCERMEACMLNGTVIGPGKTVMIDVCTTCRCMVQVG
VISGFKLECRKTTCNPCPLGYKEENNTGECCGRCLPTACTIQLRG
GQIMTLKRDETLQDGCDTHFCKVNERGEYFWEKRVTGCPPFDEHK
CLAEGGKIMKIPGTCCDTCEEPECNDITARLQYVKVGSCKSEVEV
DIHYCQGKCASKAMYSIDINDVQDQCSCCSPTRTEPMQVALHCTN
GSVVYHEVLNAMECKCSPRKCSK.

The peptide may be from the vWF A3 domain. The vWF A3 domain is derived from the human sequence, residues 1670-1874 (907-1111 of mature vWF) and has the following sequence:

(SEQ ID NO: 17)
CSGEGLQIPTLSPAPDCSQPLDVILLLDGSSSFPASYFDEMKSFAKAFI
SKANIGPRLTQVSVLQYGSITTIDVPWNVVPEKAHLLSLVDVMQREGGP
SQIGDALGFAVRYLTSEMHGARPGASKAVVILVTDVSVDSVDAAADAAR
SNRVTVFPIGIGDRYDAAQLRILAGPAGDSNVVKLQRIEDLPTMVTLGN
SFLHKLCSG.

The ECM-affinity peptide may comprise a peptide from PIGF-2. PIGF-2 has the following sequence:

(SEQ ID NO: 18)
MPVMRLFPCFLQLLAGLALPAVPPQQWALSAGNGSSEVEVVPFQEVWGR
SYCRALERLVDVVSEYPSEVEHMFSPSCVSLLRCTGCCGDENLHCVPVE
TANVTMQLLKIRSGDRPSYVELTFSQHVRCECRPLREKMKPERRRPKGR
GKRRREKQRPTDCHLCGDAVPRR.

Exemplary PIGF-2 ECM affinity peptides include:

(SEQ ID NO: 19)
RRRPKGRGKRRREKQRPTDCHLCGDAVPRR;
(SEQ ID NO: 20)
RRRPKGRGKRRREKQRPTDCHL;
(SEQ ID NO: 21)
RRPKGRGKRRREKQRPTD;
(SEQ ID NO: 22)
RRRPKGRGKRRREKQ;
(SEQ ID NO: 23)
GKRRREKQ;
(SEQ ID NO: 24)
RRRPKGRG;
and
(SEQ ID NO: 25)
RRKTKGKRKRSRNSQTEEPHP.

The ECM-affinity peptide may be a peptide from CXCL-12y. The sequence of CXCL-12Y is the following: CXCL-12y: KPVSLSYRCPCRFFESHVARANVKHLKILNTPNCALQIVARLKNNNRQVCIDPKLKW IQEYLEKALNKGRREEKVGKKEKIGKKKRQKKRKAAQKRKN (SEQ ID NO:26). An exemplary peptide includes all or part of SEQ ID NO:12 and the following peptide:

(SEQ ID NO: 27)
GRREEKVGKKEKIGKKKRQKKRKAAQKRKN.

The ECM-affinity peptide may be a peptide with 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to an ECM or CBD peptide or fragment of the peptides described above.

A linker sequence may be included in the anti-inflammatory agent-peptide construction. For example, a linker having at least, at most, or exactly 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids (or any derivable range therein) may separate that antibody and the peptide.

The ECM-affinity peptides of the disclosure may have affinity to one or more components of the extracellular matrix such as fibronectin, collagen, (collagen type I, collagen type III, and/or collagen type IV) tenascin C, fibrinogen, and fibrin. The ECM-affinity peptide may have an affinity for collagen. The ECM-affinity peptide may be one that does not bind fibronectin.

### II. Proteinaceous Compositions

The polypeptides or polynucleotides of the disclosure may include, may include at least, or may include at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOs: 1-27.

The polypeptides or polynucleotides of the disclosure may include, may include at least, or may include at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of SEQ ID NO:1-27.

A polypeptide of the disclosure may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) of SEQ ID NOs: 1-27.

A polypeptide of the disclosure may comprise, comprise at least, or comprise at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids of SEQ ID NOs: 1-27.

The polypeptides of the disclosure may comprise, may comprise at least, or may comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids of SEQ ID NOs: 1-27 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with any one of SEQ ID NOS: 1-27.

A polypeptide of the disclosure may have, have at least, or have at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any range derivable therein) sequence identity or homology with one of SEQ ID NOS: 1-27.

The disclosure includes a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 of any of SEQ ID NOS: 1-27 and comprising 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 contiguous nucleotides or amino acids of any of SEQ ID NOS: 1-27.

The polypeptides and nucleic acids of the disclosure may include, may include at least, or may include at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position or nucleic acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 of one of SEQ ID NO:1-27. One or more of these substitutions may be specifically excluded from an aspect.

The amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 of the peptide or polypeptide of one of SEQ ID NOS: 1-27 may be substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

Peptides, polypeptides, and proteins of the disclosure may have, may have at least, or may have at most 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity to any one of SEQ ID NOS: 1-27 and may includes a fragment or segment starting at amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any range derivable therein) and ending at amino acid 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, or 205 (or any range derivable therein).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa. One or more of these substitutions may be specifically excluded from an aspect.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other aspects, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific aspects, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One aspect includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### III. Nucleic Acids

In certain aspects, the current disclosure concerns recombinant polynucleotides encoding the polypeptides of the disclosure. Therefore, certain aspects relate to nucleotides encoding for polypeptides, chimeric polypeptides, or multimeric polypeptides of the disclosure.

As used in this application, the term “polynucleotide” refers to a nucleic acid molecule that either is recombinant or has been isolated free of total genomic nucleic acid. Included within the term “polynucleotide” are oligonucleotides (nucleic acids of 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence of: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs (or any range derivable therein), including all values and ranges there between, of a polynucleotide encoding one or more amino acid sequence described or referenced herein. It also is contemplated that a particular polypeptide may be encoded by nucleic acids containing variations having slightly different nucleic acid sequences but, nonetheless, encode the same or substantially similar protein.

In particular aspects, the invention concerns isolated nucleic acid segments and recombinant vectors incorporating nucleic acid sequences that encode a polypeptide or peptide of the disclosure. The term “recombinant” may be used in conjunction with a polynucleotide or polypeptide and generally refers to a polypeptide or polynucleotide produced and/or manipulated in vitro or that is a replication product of such a molecule.

In other aspects, the invention concerns isolated nucleic acid segments and recombinant vectors incorporating nucleic acid sequences that encode a polypeptide or peptide of the disclosure.

The nucleic acid segments used in the current disclosure can be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain aspects, the current disclosure provides polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence of this disclosure using the methods described herein (e.g., BLAST analysis using standard parameters).

The disclosure also contemplates the use of polynucleotides which are complementary to all the above described polynucleotides.

### A. Vectors

Polypeptides of the disclosure may be encoded by a nucleic acid molecule comprised in a vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). In addition to encoding a polypeptide of the disclosure, the vector can encode other polypeptide sequences such as a one or more other bacterial peptide, a tag, or an immunogenicity enhancing peptide. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

### B. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

Naturally, it may be important to employ a promoter and/or enhancer that effectively directs the expression of the DNA segment in the cell type or organism chosen for expression. Those of skill in the art of molecular biology generally know the use of promoters, enhancers, and cell type combinations for protein expression (see Sambrook et al., 2001, incorporated herein by reference). The promoters employed may be constitutive, tissue-specific, or inducible and in certain aspects may direct high level expression of the introduced DNA segment under specified conditions, such as large-scale production of recombinant proteins or peptides.

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

### C. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain aspects of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988; Macejak and Sarnow, 1991). IRES elements can be linked to heterologous open reading frames. Multiple open reading frames can be transcribed together, each separated by an IRES, creating polycistronic messages. Multiple genes can be efficiently expressed using a single promoter/enhancer to transcribe a single message (see U.S. Pat. Nos. 5,925,565 and 5,935,819, herein incorporated by reference).

### D. Selectable and Screenable Markers

In certain aspects of the invention, cells containing a nucleic acid construct of the current disclosure may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

### E. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org).

### F. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENER's COMPLETE CONTROL□ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### IV. Combination Therapy

The compositions and related methods of the present disclosure, particularly administration of the polypeptides of the disclosure may also be used in combination with the administration of additional therapies such as the additional therapeutics described herein or in combination with other traditional therapeutics known in the art for the treatment of autoimmune or inflammatory conditions.

The therapeutic compositions and treatments disclosed herein may precede, be co-current with and/or follow another treatment or agent by intervals ranging from minutes to weeks. In aspects where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

In some aspects, more than one course of therapy may be employed. It is contemplated that multiple courses may be implemented.

### V. Therapeutic Methods

The compositions of the disclosure may be used for in vivo, in vitro, or ex vivo administration. The route of administration of the composition may be, for example, intracutaneous, subcutaneous, intravenous, intradermal, intramuscular, local, topical, and/or intraperitoneal administrations. It is specifically contemplated that one or more of these routes of administration are excluded from certain aspects of the disclosure.

In some aspects, a composition of the disclosure is provided via subcutaneous administration (i.e., is provided subcutaneously). In some aspects, a composition of the disclosure is provided via intradermal administration (i.e., is provided intradermally). In some aspects, a composition of the disclosure is provided via intramuscular administration (i.e., is provided intramuscularly).

In some aspects, a therapeutic composition of the disclosure is administered during the cessation of one or more other therapies. For example, in some aspects, disclosed is a method comprising administering to a subject a composition of the disclosure during cessation of an additional anti-inflammatory therapeutic (e.g., fingolimod, interferon-β, dimethyl fumarate, teriflunomide, integrin α4β1, an anti-αLβ2 antibody, an anti-TNFa agent, an anti-IL-6R agent, an anti-IL-6 agent, or a Janus kinase inhibitor).

The compositions of the disclosure may be used for in vivo, in vitro, or ex vivo administration. The route of administration of the composition may be, for example, intracutaneous, subcutaneous, intravenous, local, topical, and intraperitoneal administrations.

Aspects of the present disclosure are directed to methods for treating autoimmune or inflammatory conditions. In some aspects, disclosed herein is a method for treating an autoimmune or inflammatory condition comprising administering to a subject a composition of the disclosure, where the subject has, is at risk for developing, or is suspected of having an autoimmune or inflammatory condition. Such methods may comprise administrating one or more additional anti-inflammatory agents. Such methods may exclude administering one or more additional anti-inflammatory agents. Additional anti-inflammatory agents include, for example, fingolimod, interferon-β, dimethyl fumarate, teriflunomide, integrin α4β1, an anti-αLβ2 antibody, an anti-TNFa agent, an anti-IL-6R agent, an anti-IL-6 agent, and a Janus kinase inhibitor (e.g., tofacitinib, baricitinib, upadacitinib).

The autoimmune condition or inflammatory condition amenable for treatment may include, but not be limited to conditions such as diabetes (e.g. type 1 diabetes), graft rejection, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and systemic juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes. Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, dementia, mental health disorder, depression, Alzheimer's disease, Parkinson's disease, multiple organ injury syndrome such as those secondary to septicemia, trauma or hemorrhage, antigen-antibody complex-mediated diseases, antiglomerular basement membrane disease, anti-phospholipid antibody syndrome, allergic neuritis, Bechet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopeniaurpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, liver fibrosis, kidney fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, contact hypersensitivity, asthmatic airway hyperreaction, ischemic stroke, pulmonary hypertension, adverse events of immunotherapies, atherosclerosis, wound healing, peripheral artery disease, cerebral ischemia-reperfusion (I/R) injury, myocardial infarction, and endometriosis. The compositions and polypeptides may also be used to treat inflammation associated with stroke, brain stroke, cardiac stroke, and/or acute respiratory distress syndrome. The compositions and polypeptides may also be used to treat inflammation and post-infection symptoms associated with viral infection, such as COVID, SARS, and MERS and/or administration of vaccines.

### VI. Pharmaceutical Compositions and Methods

In some aspects, pharmaceutical compositions are administered to a subject. Different aspects involve administering an effective amount of a composition to a subject. In some aspects, a composition comprising an anti-inflammatory agent may be administered to the subject or patient to treat inflammation and/or autoimmunity. Additionally, such compounds can be administered in combination with an additional treatment.

Compositions can be formulated for parenteral administration, e.g., formulated for injection via the intravenous, transcatheter injection, intraarterial injection, intramuscular, subcutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and, the preparations can also be emulsified. The preparation of such formulations will be known to those of skill in the art in light of the present disclosure. In some aspects, the administration is systemic.

Other routes of administration are also contemplated. For example, the constructs and agents may be administered in association with a carrier. In some aspects, the carrier is a nanoparticle or microparticle.

Particles can have a structure of variable dimension and known variously as a microsphere, microparticle, nanoparticle, nanosphere, or liposome. Such particulate formulations can be formed by covalent or non-covalent coupling of the construct to the particle. By “particle,” “microparticle,” “bead,” “microsphere,” and grammatical equivalents herein is meant small discrete particles that are administrable to a subject. In certain aspects, the particles are substantially spherical in shape. The term “substantially spherical,” as used herein, means that the shape of the particles does not deviate from a sphere by more than about 10%. The particles typically consist of a substantially spherical core and optionally one or more layers. The core may vary in size and composition. In addition to the core, the particle may have one or more layers to provide functionalities appropriate for the applications of interest. The thicknesses of layers, if present, may vary depending on the needs of the specific applications. For example, layers may impart useful optical properties.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. Pharmaceutically acceptable salts include the conventional non-toxic salts or the quaternary ammonium salts of the parent compound formed, for example, from non-toxic inorganic or organic acids. The pharmaceutically acceptable salts can be synthesized from the parent compound which contains a basic or acidic moiety by conventional chemical methods.

Some variation in dosage will necessarily occur depending on the condition of the subject. The person responsible for administration will, in any event, determine the appropriate dose for the individual subject. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the effects desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

Typically, for a human adult (weighing approximately 70 kilograms), from about 0.1 mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

In certain aspects, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or μg/kg or micrograms/kg/minute or mg/kg/min or micrograms/kg/hour or mg/kg/hour, or μM or mM of an agent discussed herein. Any range derivable therein is contemplated.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of a condition. In some aspects, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of the condition have disappeared or been reduced or after 6, 12, 18, or 24 hours or 1, 2, 3, 4, or 5 days after symptoms of an infection have disappeared or been reduced.

### VII. Sequences

| Description | Sequence | SEQ ID NO: |
|---|---|---|
| IL-35-HSA | RKGPPAALTLPRVQCRASRYPIAVDCSWTLPPAPNSTSPVSFIA | 91 |
| | TYRLGMAARGHSWPCLQQTPTSTSCTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESC | |
| | LNSRETSFITNGSCLASRKTSFMMALCLSSIYEDLKMYQVEFK | |
| | TMNAKLLMDPKRQIFLDQNMLAVIDELMQALNFNSETVPQK | |
| | SSLEEPDFYKTKIKLCILLHAFRIRAVTIDRVMSYLNASGGGSG | |
| | GGSGGGSGGGSGGGSDAHKSEVAHRFKDLGEENFKALVLIAF | |
| | AQYLQQCPFEDHVKLVNEVTEFAKTCVADESAENCDKSLHT | |
| | LFGDKLCTVATLRETYGEMADCCAKQEPERNECFLQHKDDN | |
| | PNLPRLVRPEVDVMCTAFHDNEETFLKKYLYEIARRHPYFYA | |
| | PELLFFAKRYKAAFTECCQAADKAACLLPKLDELRDEGKASS | |
| | AKQRLKCASLQKFGERAFKAWAVARLSQRFPKAEFAEVSKL | |
| | VTDLTKVHTECCHGDLLECADDRADLAKYICENQDSISSKLK | |
| | ECCEKPLLEKSHCIAEVENDEMPADLPSLAADFVESKDVCKN | |
| | YAEAKDVFLGMFLYEYARRHPDYSVVLLLRLAKTYETTLEK | |
| | CCAAADPHECYAKVFDEFKPLVEEPQNLIKQNCELFEQLGEY | |
| | KFQNALLVRYTKKVPQVSTPTLVEVSRNLGKVGSKCCKHPEA | |
| | KRMPCAEDYLSVVLNQLCVLHEKTPVSDRVTKCCTESLVNR | |
| | RPCFSALEVDETYVPKEFNAETFTFHADICTLSEKERQIKKQT | |
| | ALVELVKHKPKATKEQLKAVMDDFAAFVEKCCKADDKETCF | |
| | AEEGKKLVAASQAALGL | |
| Signal | METDTLLLWVLLLWVPGSTGDRKGPPAALTLPRVQCRASRY | 1 |
| Peptide-IL- | PIAVDCSWTLPPAPNSTSPVSFIATYRLGMAARGHSWPCLQQT | |
| 35-HSA | PTSTSCTITDVQLFSMAPYVLNVTAVHPWGSSSSFVPFITEHIIK | |
| | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESCLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASGGGSGGGSGGGSGGGSGGGSDAHKSE | |
| | VAHRFKDLGEENFKALVLIAFAQYLQQCPFEDHVKLVNEVTE | |
| | FAKTCVADESAENCDKSLHTLFGDKLCTVATLRETYGEMAD | |
| | CCAKQEPERNECFLQHKDDNPNLPRLVRPEVDVMCTAFHDN | |
| | EETFLKKYLYEIARRHPYFYAPELLFFAKRYKAAFTECCQAA | |
| | DKAACLLPKLDELRDEGKASSAKQRLKCASLQKFGERAFKA | |
| | WAVARLSQRFPKAEFAEVSKLVTDLTKVHTECCHGDLLECA | |
| | DDRADLAKYICENQDSISSKLKECCEKPLLEKSHCIAEVENDE | |
| | MPADLPSLAADFVESKDVCKNYAEAKDVFLGMFLYEYARRH | |
| | PDYSVVLLLRLAKTYETTLEKCCAAADPHECYAKVFDEFKPL | |
| | VEEPQNLIKQNCELFEQLGEYKFQNALLVRYTKKVPQVSTPT | |
| | LVEVSRNLGKVGSKCCKHPEAKRMPCAEDYLSVVLNQLCVL | |
| | HEKTPVSDRVTKCCTESLVNRRPCFSALEVDETYVPKEFNAET | |
| | FTFHADICTLSEKERQIKKQTALVELVKHKPKATKEQLKAVM | |
| | DDFAAFVEKCCKADDKETCFAEEGKKLVAASQAALGL | |
| Signal | METDTLLLWVLLLWVPGSTGDRKGPPAALTLPRVQCRASRY | 2 |
| peptide- | PIAVDCSWTLPPAPNSTSPVSFIATYRLGMAARGHSWPCLQQT | |
| EBI3 | PTSTSCTITDVQLFSMAPYVLNVTAVHPWGSSSSFVPFITEHIIK | |
| | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGK | |
| Linker | GGGSGGGSGGGSGGGS | 3 |
| P35 | RNLPVATPDPGMFPCLHHSQNLLRAVSNMLQKARQTLEFYPC | 4 |
| | TSEEIDHEDITKDKTSTVEACLPLELTKNESCLNSRETSFITNGS | |
| | CLASRKTSFMMALCLSSIYEDLKMYQVEFKTMNAKLLMDPK | |
| | RQIFLDQNMLAVIDELMQALNFNSETVPQKSSLEEPDFYKTKI | |
| | KLCILLHAFRIRAVTIDRVMSYLNAS | |
| Linker | GGGSGGGSGGGSGGGSGGGS | 5 |
| HSA | DAHKSEVAHRFKDLGEENFKALVLIAFAQYLQQCPFEDHVKL | 6 |
| | VNEVTEFAKTCVADESAENCDKSLHTLFGDKLCTVATLRETY | |
| | GEMADCCAKQEPERNECFLQHKDDNPNLPRLVRPEVDVMCT | |
| | AFHDNEETFLKKYLYEIARRHPYFYAPELLFFAKRYKAAFTEC | |
| | CQAADKAACLLPKLDELRDEGKASSAKQRLKCASLQKFGER | |
| | AFKAWAVARLSQRFPKAEFAEVSKLVTDLTKVHTECCHGDL | |
| | LECADDRADLAKYICENQDSISSKLKECCEKPLLEKSHCIAEV | |
| | ENDEMPADLPSLAADFVESKDVCKNYAEAKDVFLGMFLYEY | |
| | ARRHPDYSVVLLLRLAKTYETTLEKCCAAADPHECYAKVFD | |
| | EFKPLVEEPQNLIKQNCELFEQLGEYKFQNALLVRYTKKVPQ | |
| | VSTPTLVEVSRNLGKVGSKCCKHPEAKRMPCAEDYLSVVLN | |
| | QLCVLHEKTPVSDRVTKCCTESLVNRRPCFSALEVDETYVPK | |
| | EFNAETFTFHADICTLSEKERQIKKQTALVELVKHKPKATKEQ | |
| | LKAVMDDFAAFVEKCCKADDKETCFAEEGKKLVAASQAAL | |
| | GL | |
| Signal | METDTLLLWVLLLWVPGSTGD | 89 |
| Peptide | | |
| EBI3 | RKGPPAALTLPRVQCRASRYPIAVDCSWTLPPAPNSTSPVSFIA | 90 |
| | TYRLGMAARGHSWPCLQQTPTSTSCTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGK | |
| Delta20 IL- | PIAVDCSWTLPPAPNSTSPVSFIATYRLGMAARGHSWPCLQQT | 28 |
| 35 | PTSTSCTITDVQLFSMAPYVLNVTAVHPWGSSSSFVPFITEHIIK | |
| | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNAS | |
| delta20 IL- | PIAVDCSWTLPPAPNSTSPVSFIATYRLGMAARGHSWPCLQQT | 29 |
| 35-HSA | PTSTSCTITDVQLFSMAPYVLNVTAVHPWGSSSSFVPFITEHIIK | |
| | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASGGGSGGGSGGGSGGGSGGGSDAHKSE | |
| | VAHRFKDLGEENFKALVLIAFAQYLQQAPFEDHVKLVNEVTE | |
| | FAKTCVADESAENCDKSLHTLFGDKLCTVATLRETYGEMAD | |
| | CCAKQEPERNECFLQHKDDNPNLPRLVRPEVDVMCTAFHDN | |
| | EETFLKKYLYEIARRHPYFYAPELLFFAKRYKAAFTECCQAA | |
| | DKAACLLPKLDELRDEGKASSAKQRLKCASLQKFGERAFKA | |
| | WAVARLSQRFPKAEFAEVSKLVTDLTKVHTECCHGDLLECA | |
| | DDRADLAKYICENQDSISSKLKECCEKPLLEKSHCIAEVENDE | |
| | MPADLPSLAADFVESKDVCKNYAEAKDVFLGMFLYEYARRH | |
| | PDYSVVLLLRLAKTYETTLEKCCAAADPHECYAKVFDEFKPL | |
| | VEEPQNLIKQNCELFEQLGEYKFQNALLVRYTKKVPQVSTPT | |
| | LVEVSRNLGKVGSKCCKHPEAKRMPCAEDYLSVVLNQLCVL | |
| | HEKTPVSDRVTKCCTESLVNRRPCFSALEVDETYVPKEFNAET | |
| | FTFHADICTLSEKERQIKKQTALVELVKHKPKATKEQLKAVM | |
| | DDFAAFVEKCCKADDKETCFAEEGKKLVAASQAALGL | |
| delta10 IL- | PRVQCRASRYPIAVDCSWTLPPAPNSTSPVSFIATYRLGMAAR | 30 |
| 35-HSA | GHSWPCLQQTPTSTSCTITDVQLFSMAPYVLNVTAVHPWGSS | |
| | SSFVPFITEHIIKPDPPEGVRLSPLAERQLQVQWEPPGSWPFPEI | |
| | FSLKYWIRYKRQGAARFHRVGPIEATSFILRAVRPRARYYVQ | |
| | VAAQDLTDYGELSDWSLPATATMSLGKGGGSGGGSGGGSGG | |
| | GSRNLPVATPDPGMFPCLHHSQNLLRAVSNMLQKARQTLEFY | |
| | PCTSEEIDHEDITKDKTSTVEACLPLELTKNESSLNSRETSFITN | |
| | GSCLASRKTSFMMALCLSSIYEDLKMYQVEFKTMNAKLLMD | |
| | PKRQIFLDQNMLAVIDELMQALNFNSETVPQKSSLEEPDFYKT | |
| | KIKLCILLHAFRIRAVTIDRVMSYLNASGGGSGGGSGGGSGGG | |
| | SGGGSDAHKSEVAHRFKDLGEENFKALVLIAFAQYLQQAPFE | |
| | DHVKLVNEVTEFAKTCVADESAENCDKSLHTLFGDKLCTVA | |
| | TLRETYGEMADCCAKQEPERNECFLQHKDDNPNLPRLVRPEV | |
| | DVMCTAFHDNEETFLKKYLYEIARRHPYFYAPELLFFAKRYK | |
| | AAFTECCQAADKAACLLPKLDELRDEGKASSAKQRLKCASL | |
| | QKFGERAFKAWAVARLSQRFPKAEFAEVSKLVTDLTKVHTE | |
| | CCHGDLLECADDRADLAKYICENQDSISSKLKECCEKPLLEKS | |
| | HCIAEVENDEMPADLPSLAADFVESKDVCKNYAEAKDVFLG | |
| | MFLYEYARRHPDYSVVLLLRLAKTYETTLEKCCAAADPHEC | |
| | YAKVFDEFKPLVEEPQNLIKQNCELFEQLGEYKFQNALLVRY | |
| | TKKVPQVSTPTLVEVSRNLGKVGSKCCKHPEAKRMPCAEDY | |
| | LSVVLNQLCVLHEKTPVSDRVTKCCTESLVNRRPCFSALEVD | |
| | ETYVPKEFNAETFTFHADICTLSEKERQIKKQTALVELVKHKP | |
| | KATKEQLKAVMDDFAAFVEKCCKADDKETCFAEEGKKLVA | |
| | ASQAALGL | |
| Delta20 IL- | PIAVDCSWTLPPAPNSTSPVSFIATYRLGMAARGHSWPCLQQT | 31 |
| 35-Fc | PTSTSCTITDVQLESMAPYVLNVTAVHPWGSSSSFVPFITEHIIK | |
| | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASEPKSCDKTHTCPPCPAPEFEGGPSVFLF | |
| | PPKPKDTLMISRTPEVTCVVVDVSHEDPEVKFNWYVDGVEV | |
| | HNAKTKPREEQYASTYRVVSVLTVLHQDWLNGKEYKCKVS | |
| | NKALPASIEKTISKAKGQPREPQVYTLPPSRDELTKNQVSLTC | |
| | LVKGFYPSDIAVEWESNGQPENNYKTTPPVLDSDGSFFLYSKL | |
| | TVDKSRWQQGNVFSCSVLHEALHSHYTQKSLSLSPGK | |
| Delta10 IL- | PRVQCRASRYPIAVDCSWTLPPAPNSTSPVSFIATYRLGMAAR | 32 |
| 35-Fc | GHSWPCLQQTPTSTSCTITDVQLFSMAPYVLNVTAVHPWGSS | |
| | SSFVPFITEHIIKPDPPEGVRLSPLAERQLQVQWEPPGSWPFPEI | |
| | FSLKYWIRYKRQGAARFHRVGPIEATSFILRAVRPRARYYVQ | |
| | VAAQDLTDYGELSDWSLPATATMSLGKGGGSGGGSGGGSGG | |
| | GSRNLPVATPDPGMFPCLHHSQNLLRAVSNMLQKARQTLEFY | |
| | PCTSEEIDHEDITKDKTSTVEACLPLELTKNESSLNSRETSFITN | |
| | GSCLASRKTSFMMALCLSSIYEDLKMYQVEFKTMNAKLLMD | |
| | PKRQIFLDQNMLAVIDELMQALNFNSETVPQKSSLEEPDFYKT | |
| | KIKLCILLHAFRIRAVTIDRVMSYLNASEPKSCDKTHTCPPCPA | |
| | PEFEGGPSVFLFPPKPKDTLMISRTPEVTCVVVDVSHEDPEVK | |
| | FNWYVDGVEVHNAKTKPREEQYASTYRVVSVLTVLHQDWL | |
| | NGKEYKCKVSNKALPASIEKTISKAKGQPREPQVYTLPPSRDE | |
| | LTKNQVSLTCLVKGFYPSDIAVEWESNGQPENNYKTTPPVLD | |
| | SDGSFFLYSKLTVDKSRWQQGNVFSCSVLHEALHSHYTQKSL | |
| | SLSPGK | |
| IL-35 1Ser | RAGPPAALTLPRVQSRASRYPIAVDCSWTLPPAPNSTSPVSFIA | 33 |
| | TYRLGMAARGHSWPCLQQTPTSTSCTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESS | |
| | LNSRETSFITNGSCLASRKTSFMMALCLSSIYEDLKMYQVEFK | |
| | TMNAKLLMDPKRQIFLDQNMLAVIDELMQALNENSETVPQK | |
| | SSLEEPDFYKTKIKLCILLHAFRIRAVTIDRVMSYLNAS | |
| IL-35 2Ser | RAGPPAALTLPRVQSRASRYPIAVDSSWTLPPAPNSTSPVSFIA | 34 |
| | TYRLGMAARGHSWPCLQQTPTSTSCTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESS | |
| | LNSRETSFITNGSCLASRKTSFMMALCLSSIYEDLKMYQVEFK | |
| | TMNAKLLMDPKRQIFLDQNMLAVIDELMQALNENSETVPQK | |
| | SSLEEPDFYKTKIKLCILLHAFRIRAVTIDRVMSYLNAS | |
| IL-35 3Ser | RAGPPAALTLPRVQSRASRYPIAVDSSWTLPPAPNSTSPVSFIA | 35 |
| | TYRLGMAARGHSWPSLQQTPTSTSCTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESS | |
| | LNSRETSFITNGSCLASRKTSFMMALCLSSIYEDLKMYQVEFK | |
| | TMNAKLLMDPKRQIFLDQNMLAVIDELMQALNFNSETVPQK | |
| | SSLEEPDFYKTKIKLCILLHAFRIRAVTIDRVMSYLNAS | |
| IL-35 4Ser | RAGPPAALTLPRVQSRASRYPIAVDSSWTLPPAPNSTSPVSFIA | 36 |
| | TYRLGMAARGHSWPSLQQTPTSTSSTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESS | |
| | LNSRETSFITNGSCLASRKTSFMMALCLSSIYEDLKMYQVEFK | |
| | TMNAKLLMDPKRQIFLDQNMLAVIDELMQALNFNSETVPQK | |
| | SSLEEPDFYKTKIKLCILLHAFRIRAVTIDRVMSYLNAS | |
| IL-35 1Ser- | RAGPPAALTLPRVQSRASRYPIAVDCSWTLPPAPNSTSPVSFIA | 37 |
| HSA | TYRLGMAARGHSWPCLQQTPTSTSCTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESS | |
| | LNSRETSFITNGSCLASRKTSFMMALCLSSIYEDLKMYQVEFK | |
| | TMNAKLLMDPKRQIFLDQNMLAVIDELMQALNFNSETVPQK | |
| | SSLEEPDFYKTKIKLCILLHAFRIRAVTIDRVMSYLNASGGGSG | |
| | GGSGGGSGGGSGGGSDAHKSEVAHRFKDLGEENFKALVLIAF | |
| | AQYLQQAPFEDHVKLVNEVTEFAKTCVADESAENCDKSLHT | |
| | LFGDKLCTVATLRETYGEMADCCAKQEPERNECFLQHKDDN | |
| | PNLPRLVRPEVDVMCTAFHDNEETFLKKYLYEIARRHPYFYA | |
| | PELLFFAKRYKAAFTECCQAADKAACLLPKLDELRDEGKASS | |
| | AKQRLKCASLQKFGERAFKAWAVARLSQRFPKAEFAEVSKL | |
| | VTDLTKVHTECCHGDLLECADDRADLAKYICENQDSISSKLK | |
| | ECCEKPLLEKSHCIAEVENDEMPADLPSLAADFVESKDVCKN | |
| | YAEAKDVFLGMFLYEYARRHPDYSVVLLLRLAKTYETTLEK | |
| | CCAAADPHECYAKVFDEFKPLVEEPQNLIKQNCELFEQLGEY | |
| | KFQNALLVRYTKKVPQVSTPTLVEVSRNLGKVGSKCCKHPEA | |
| | KRMPCAEDYLSVVLNQLCVLHEKTPVSDRVTKCCTESLVNR | |
| | RPCFSALEVDETYVPKEFNAETFTFHADICTLSEKERQIKKQT | |
| | ALVELVKHKPKATKEQLKAVMDDFAAFVEKCCKADDKETCF | |
| | AEEGKKLVAASQAALGL | |
| IL-35 2Ser- | RAGPPAALTLPRVQSRASRYPIAVDSSWTLPPAPNSTSPVSFIA | 38 |
| HSA | TYRLGMAARGHSWPCLQQTPTSTSCTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESS | |
| | LNSRETSFITNGSCLASRKTSFMMALCLSSIYEDLKMYQVEFK | |
| | TMNAKLLMDPKRQIFLDQNMLAVIDELMQALNFNSETVPQK | |
| | SSLEEPDFYKTKIKLCILLHAFRIRAVTIDRVMSYLNASGGGSG | |
| | GGSGGGSGGGSGGGSDAHKSEVAHRFKDLGEENFKALVLIAF | |
| | AQYLQQAPFEDHVKLVNEVTEFAKTCVADESAENCDKSLHT | |
| | LFGDKLCTVATLRETYGEMADCCAKQEPERNECFLQHKDDN | |
| | PNLPRLVRPEVDVMCTAFHDNEETFLKKYLYEIARRHPYFYA | |
| | PELLFFAKRYKAAFTECCQAADKAACLLPKLDELRDEGKASS | |
| | AKQRLKCASLQKFGERAFKAWAVARLSQRFPKAEFAEVSKL | |
| | VTDLTKVHTECCHGDLLECADDRADLAKYICENQDSISSKLK | |
| | ECCEKPLLEKSHCIAEVENDEMPADLPSLAADFVESKDVCKN | |
| | YAEAKDVFLGMFLYEYARRHPDYSVVLLLRLAKTYETTLEK | |
| | CCAAADPHECYAKVFDEFKPLVEEPQNLIKQNCELFEQLGEY | |
| | KFQNALLVRYTKKVPQVSTPTLVEVSRNLGKVGSKCCKHPEA | |
| | KRMPCAEDYLSVVLNQLCVLHEKTPVSDRVTKCCTESLVNR | |
| | RPCFSALEVDETYVPKEFNAETFTFHADICTLSEKERQIKKQT | |
| | ALVELVKHKPKATKEQLKAVMDDFAAFVEKCCKADDKETCF | |
| | AEEGKKLVAASQAALGL | |
| IL-35 3Ser- | RAGPPAALTLPRVQSRASRYPIAVDSSWTLPPAPNSTSPVSFIA | 39 |
| HSA | TYRLGMAARGHSWPSLQQTPTSTSCTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESS | |
| | LNSRETSFITNGSCLASRKTSFMMALCLSSIYEDLKMYQVEFK | |
| | TMNAKLLMDPKRQIFLDQNMLAVIDELMQALNFNSETVPQK | |
| | SSLEEPDFYKTKIKLCILLHAFRIRAVTIDRVMSYLNASGGGSG | |
| | GGSGGGSGGGSGGGSDAHKSEVAHRFKDLGEENFKALVLIAF | |
| | AQYLQQAPFEDHVKLVNEVTEFAKTCVADESAENCDKSLHT | |
| | LFGDKLCTVATLRETYGEMADCCAKQEPERNECFLQHKDDN | |
| | PNLPRLVRPEVDVMCTAFHDNEETFLKKYLYEIARRHPYFYA | |
| | PELLFFAKRYKAAFTECCQAADKAACLLPKLDELRDEGKASS | |
| | AKQRLKCASLQKFGERAFKAWAVARLSQRFPKAEFAEVSKL | |
| | VTDLTKVHTECCHGDLLECADDRADLAKYICENQDSISSKLK | |
| | ECCEKPLLEKSHCIAEVENDEMPADLPSLAADFVESKDVCKN | |
| | YAEAKDVFLGMFLYEYARRHPDYSVVLLLRLAKTYETTLEK | |
| | CCAAADPHECYAKVFDEFKPLVEEPQNLIKQNCELFEQLGEY | |
| | KFQNALLVRYTKKVPQVSTPTLVEVSRNLGKVGSKCCKHPEA | |
| | KRMPCAEDYLSVVLNQLCVLHEKTPVSDRVTKCCTESLVNR | |
| | RPCFSALEVDETYVPKEFNAETFTFHADICTLSEKERQIKKQT | |
| | ALVELVKHKPKATKEQLKAVMDDFAAFVEKCCKADDKETCF | |
| | AEEGKKLVAASQAALGL | |
| IL-35 4Ser- | RAGPPAALTLPRVQSRASRYPIAVDSSWTLPPAPNSTSPVSFIA | 40 |
| HSA | TYRLGMAARGHSWPSLQQTPTSTSSTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESS | |
| | LNSRETSFITNGSCLASRKTSFMMALCLSSIYEDLKMYQVEFK | |
| | TMNAKLLMDPKRQIFLDQNMLAVIDELMQALNFNSETVPQK | |
| | SSLEEPDFYKTKIKLCILLHAFRIRAVTIDRVMSYLNASGGGSG | |
| | GGSGGGSGGGSGGGSDAHKSEVAHRFKDLGEENFKALVLIAF | |
| | AQYLQQAPFEDHVKLVNEVTEFAKTCVADESAENCDKSLHT | |
| | LFGDKLCTVATLRETYGEMADCCAKQEPERNECFLQHKDDN | |
| | PNLPRLVRPEVDVMCTAFHDNEETFLKKYLYEIARRHPYFYA | |
| | PELLFFAKRYKAAFTECCQAADKAACLLPKLDELRDEGKASS | |
| | AKQRLKCASLQKFGERAFKAWAVARLSQRFPKAEFAEVSKL | |
| | VTDLTKVHTECCHGDLLECADDRADLAKYICENQDSISSKLK | |
| | ECCEKPLLEKSHCIAEVENDEMPADLPSLAADFVESKDVCKN | |
| | YAEAKDVFLGMFLYEYARRHPDYSVVLLLRLAKTYETTLEK | |
| | CCAAADPHECYAKVFDEFKPLVEEPQNLIKQNCELFEQLGEY | |
| | KFQNALLVRYTKKVPQVSTPTLVEVSRNLGKVGSKCCKHPEA | |
| | KRMPCAEDYLSVVLNQLCVLHEKTPVSDRVTKCCTESLVNR | |
| | RPCFSALEVDETYVPKEFNAETFTFHADICTLSEKERQIKKQT | |
| | ALVELVKHKPKATKEQLKAVMDDFAAFVEKCCKADDKETCF | |
| | AEEGKKLVAASQAALGL | |
| IL-35 1Ser- | RAGPPAALTLPRVQSRASRYPIAVDCSWTLPPAPNSTSPVSFIA | 41 |
| Fc | TYRLGMAARGHSWPCLQQTPTSTSCTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESS | |
| | LNSRETSFITNGSCLASRKTSFMMALCLSSIYEDLKMYQVEFK | |
| | TMNAKLLMDPKRQIFLDQNMLAVIDELMQALNFNSETVPQK | |
| | SSLEEPDFYKTKIKLCILLHAFRIRAVTIDRVMSYLNASEPKSC | |
| | DKTHTCPPCPAPEFEGGPSVFLFPPKPKDTLMISRTPEVTCVVV | |
| | DVSHEDPEVKFNWYVDGVEVHNAKTKPREEQYASTYRVVSV | |
| | LTVLHQDWLNGKEYKCKVSNKALPASIEKTISKAKGQPREPQ | |
| | VYTLPPSRDELTKNQVSLTCLVKGFYPSDIAVEWESNGQPEN | |
| | NYKTTPPVLDSDGSFFLYSKLTVDKSRWQQGNVFSCSVLHEA | |
| | LHSHYTQKSLSLSPGK | |
| IL-35 2Ser- | RAGPPAALTLPRVQSRASRYPIAVDSSWTLPPAPNSTSPVSFIA | 42 |
| Fc | TYRLGMAARGHSWPCLQQTPTSTSCTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESS | |
| | LNSRETSFITNGSCLASRKTSFMMALCLSSIYEDLKMYQVEFK | |
| | TMNAKLLMDPKRQIFLDQNMLAVIDELMQALNFNSETVPQK | |
| | SSLEEPDFYKTKIKLCILLHAFRIRAVTIDRVMSYLNASEPKSC | |
| | DKTHTCPPCPAPEFEGGPSVFLFPPKPKDTLMISRTPEVTCVVV | |
| | DVSHEDPEVKFNWYVDGVEVHNAKTKPREEQYASTYRVVSV | |
| | LTVLHQDWLNGKEYKCKVSNKALPASIEKTISKAKGQPREPQ | |
| | VYTLPPSRDELTKNQVSLTCLVKGFYPSDIAVEWESNGQPEN | |
| | NYKTTPPVLDSDGSFFLYSKLTVDKSRWQQGNVFSCSVLHEA | |
| | LHSHYTQKSLSLSPGK | |
| IL-35 3Ser- | RAGPPAALTLPRVQSRASRYPIAVDSSWTLPPAPNSTSPVSFIA | 43 |
| Fc | TYRLGMAARGHSWPSLQQTPTSTSCTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESS | |
| | LNSRETSFITNGSCLASRKTSFMMALCLSSIYEDLKMYQVEFK | |
| | TMNAKLLMDPKRQIFLDQNMLAVIDELMQALNFNSETVPQK | |
| | SSLEEPDFYKTKIKLCILLHAFRIRAVTIDRVMSYLNASEPKSC | |
| | DKTHTCPPCPAPEFEGGPSVFLFPPKPKDTLMISRTPEVTCVVV | |
| | DVSHEDPEVKFNWYVDGVEVHNAKTKPREEQYASTYRVVSV | |
| | LTVLHQDWLNGKEYKCKVSNKALPASIEKTISKAKGQPREPQ | |
| | VYTLPPSRDELTKNQVSLTCLVKGFYPSDIAVEWESNGQPEN | |
| | NYKTTPPVLDSDGSFFLYSKLTVDKSRWQQGNVFSCSVLHEA | |
| | LHSHYTQKSLSLSPGK | |
| IL-35 4Ser- | RAGPPAALTLPRVQSRASRYPIAVDSSWTLPPAPNSTSPVSFIA | 44 |
| Fc | TYRLGMAARGHSWPSLQQTPTSTSSTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESS | |
| | LNSRETSFITNGSCLASRKTSFMMALCLSSIYEDLKMYQVEFK | |
| | TMNAKLLMDPKRQIFLDQNMLAVIDELMQALNENSETVPQK | |
| | SSLEEPDFYKTKIKLCILLHAFRIRAVTIDRVMSYLNASEPKSC | |
| | DKTHTCPPCPAPEFEGGPSVFLFPPKPKDTLMISRTPEVTCVVV | |
| | DVSHEDPEVKFNWYVDGVEVHNAKTKPREEQYASTYRVVSV | |
| | LTVLHQDWLNGKEYKCKVSNKALPASIEKTISKAKGQPREPQ | |
| | VYTLPPSRDELTKNQVSLTCLVKGFYPSDIAVEWESNGQPEN | |
| | NYKTTPPVLDSDGSFFLYSKLTVDKSRWQQGNVFSCSVLHEA | |
| | LHSHYTQKSLSLSPGK | |
| Delta20 IL- | PIAVDSSWTLPPAPNSTSPVSFIATYRLGMAARGHSWPCLQQT | 45 |
| 35 1Ser-Fc | PTSTSCTITDVQLESMAPYVLNVTAVHPWGSSSSFVPFITEHIIK | |
| | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASEPKSCDKTHTCPPCPAPEFEGGPSVFLF | |
| | PPKPKDTLMISRTPEVTCVVVDVSHEDPEVKFNWYVDGVEV | |
| | HNAKTKPREEQYASTYRVVSVLTVLHQDWLNGKEYKCKVS | |
| | NKALPASIEKTISKAKGQPREPQVYTLPPSRDELTKNQVSLTC | |
| | LVKGFYPSDIAVEWESNGQPENNYKTTPPVLDSDGSFFLYSKL | |
| | TVDKSRWQQGNVFSCSVLHEALHSHYTQKSLSLSPGK | |
| Delta20 IL- | PIAVDSSWTLPPAPNSTSPVSFIATYRLGMAARGHSWPSLQQT | 46 |
| 35 2Ser-Fc | PTSTSCTITDVQLFSMAPYVLNVTAVHPWGSSSSFVPFITEHIIK | |
| | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASEPKSCDKTHTCPPCPAPEFEGGPSVFLF | |
| | PPKPKDTLMISRTPEVTCVVVDVSHEDPEVKFNWYVDGVEV | |
| | HNAKTKPREEQYASTYRVVSVLTVLHQDWLNGKEYKCKVS | |
| | NKALPASIEKTISKAKGQPREPQVYTLPPSRDELTKNQVSLTC | |
| | LVKGFYPSDIAVEWESNGQPENNYKTTPPVLDSDGSFFLYSKL | |
| | TVDKSRWQQGNVFSCSVLHEALHSHYTQKSLSLSPGK | |
| Delta20 IL- | PIAVDSSWTLPPAPNSTSPVSFIATYRLGMAARGHSWPSLQQT | 47 |
| 35 3Ser-Fc | PTSTSSTITDVQLFSMAPYVLNVTAVHPWGSSSSFVPFITEHIIK | |
| | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASEPKSCDKTHTCPPCPAPEFEGGPSVFLF | |
| | PPKPKDTLMISRTPEVTCVVVDVSHEDPEVKFNWYVDGVEV | |
| | HNAKTKPREEQYASTYRVVSVLTVLHQDWLNGKEYKCKVS | |
| | NKALPASIEKTISKAKGQPREPQVYTLPPSRDELTKNQVSLTC | |
| | LVKGFYPSDIAVEWESNGQPENNYKTTPPVLDSDGSFFLYSKL | |
| | TVDKSRWQQGNVFSCSVLHEALHSHYTQKSLSLSPGK | |
| delta20 | PIAVDCSWTLPPAPNSTSPVSFIATYRLGMAARGHSWPSLQQT | 48 |
| 1ser IL-35 | PTSTSCTITDVQLESMAPYVLNVTAVHPWGSSSSFVPFITEHIIK | |
| | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNAS | |
| delta20 | PIAVDCSWTLPPAPNSTSPVSFIATYRLGMAARGHSWPSLQQT | 49 |
| 2ser IL-35 | PTSTSSTITDVQLFSMAPYVLNVTAVHPWGSSSSFVPFITEHIIK | |
| | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNAS | |
| delta20 | PIAVDSSWTLPPAPNSTSPVSFIATYRLGMAARGHSWPSLQQT | 50 |
| 3ser hIL-35 | PTSTSSTITDVQLFSMAPYVLNVTAVHPWGSSSSFVPFITEHIIK | |
| | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNAS | |
| delta20 | PIAVDCSWTLPPAPNSTSPVSFIATYRLGMAARGHSWPSLQQT | 51 |
| 1ser IL-35- | PTSTSCTITDVQLFSMAPYVLNVTAVHPWGSSSSFVPFITEHIIK | |
| HSA | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASGGGSGGGSGGGSGGGSGGGSDAHKSE | |
| | VAHRFKDLGEENFKALVLIAFAQYLQQAPFEDHVKLVNEVTE | |
| | FAKTCVADESAENCDKSLHTLFGDKLCTVATLRETYGEMAD | |
| | CCAKQEPERNECFLQHKDDNPNLPRLVRPEVDVMCTAFHDN | |
| | EETFLKKYLYEIARRHPYFYAPELLFFAKRYKAAFTECCQAA | |
| | DKAACLLPKLDELRDEGKASSAKQRLKCASLQKFGERAFKA | |
| | WAVARLSQRFPKAEFAEVSKLVTDLTKVHTECCHGDLLECA | |
| | DDRADLAKYICENQDSISSKLKECCEKPLLEKSHCIAEVENDE | |
| | MPADLPSLAADFVESKDVCKNYAEAKDVFLGMFLYEYARRH | |
| | PDYSVVLLLRLAKTYETTLEKCCAAADPHECYAKVFDEFKPL | |
| | VEEPQNLIKQNCELFEQLGEYKFQNALLVRYTKKVPQVSTPT | |
| | LVEVSRNLGKVGSKCCKHPEAKRMPCAEDYLSVVLNQLCVL | |
| | HEKTPVSDRVTKCCTESLVNRRPCFSALEVDETYVPKEFNAET | |
| | FTFHADICTLSEKERQIKKQTALVELVKHKPKATKEQLKAVM | |
| | DDFAAFVEKCCKADDKETCFAEEGKKLVAASQAALGL | |
| delta20 | PIAVDSSWTLPPAPNSTSPVSFIATYRLGMAARGHSWPSLQQT | 52 |
| 3ser IL-35- | PTSTSSTITDVQLFSMAPYVLNVTAVHPWGSSSSFVPFITEHIIK | |
| HSA | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASGGGSGGGSGGGSGGGSGGGSDAHKSE | |
| | VAHRFKDLGEENFKALVLIAFAQYLQQAPFEDHVKLVNEVTE | |
| | FAKTCVADESAENCDKSLHTLFGDKLCTVATLRETYGEMAD | |
| | CCAKQEPERNECFLQHKDDNPNLPRLVRPEVDVMCTAFHDN | |
| | EETFLKKYLYEIARRHPYFYAPELLFFAKRYKAAFTECCQAA | |
| | DKAACLLPKLDELRDEGKASSAKQRLKCASLQKFGERAFKA | |
| | WAVARLSQRFPKAEFAEVSKLVTDLTKVHTECCHGDLLECA | |
| | DDRADLAKYICENQDSISSKLKECCEKPLLEKSHCIAEVENDE | |
| | MPADLPSLAADFVESKDVCKNYAEAKDVFLGMFLYEYARRH | |
| | PDYSVVLLLRLAKTYETTLEKCCAAADPHECYAKVFDEFKPL | |
| | VEEPQNLIKQNCELFEQLGEYKFQNALLVRYTKKVPQVSTPT | |
| | LVEVSRNLGKVGSKCCKHPEAKRMPCAEDYLSVVLNQLCVL | |
| | HEKTPVSDRVTKCCTESLVNRRPCFSALEVDETYVPKEFNAET | |
| | FTFHADICTLSEKERQIKKQTALVELVKHKPKATKEQLKAVM | |
| | DDFAAFVEKCCKADDKETCFAEEGKKLVAASQAALGL | |
| CBD-IL- | CSQPLDVILLLDGSSSFPASYFDEMKSFAKAFISKANIGPRLTQ | 53 |
| 35-HSA | VSVLQYGSITTIDVPWNVVPEKAHLLSLVDVMQREGGPSQIG | |
| | DALGFAVRYLTSEMHGARPGASKAVVILVTDVSVDSVDAAA | |
| | DAARSNRVTVFPIGIGDRYDAAQLRILAGPAGDSNVVKLQRIE | |
| | DLPTMVTLGNSFLHKLCSGFVRIGSRAGPPAALTLPRVQCRAS | |
| | RYPIAVDCSWTLPPAPNSTSPVSFIATYRLGMAARGHSWPCLQ | |
| | QTPTSTSCTITDVQLFSMAPYVLNVTAVHPWGSSSSFVPFITEH | |
| | IIKPDPPEGVRLSPLAERQLQVQWEPPGSWPFPEIFSLKYWIRY | |
| | KRQGAARFHRVGPIEATSFILRAVRPRARYYVQVAAQDLTDY | |
| | GELSDWSLPATATMSLGKGGGSGGGSGGGSGGGSRNLPVAT | |
| | PDPGMFPCLHHSQNLLRAVSNMLQKARQTLEFYPCTSEEIDH | |
| | EDITKDKTSTVEACLPLELTKNESSLNSRETSFITNGSCLASRK | |
| | TSFMMALCLSSIYEDLKMYQVEFKTMNAKLLMDPKRQIFLD | |
| | QNMLAVIDELMQALNFNSETVPQKSSLEEPDFYKTKIKLCILL | |
| | HAFRIRAVTIDRVMSYLNASGGGSGGGSGGGSGGGSGGGSD | |
| | AHKSEVAHRFKDLGEENFKALVLIAFAQYLQQAPFEDHVKLV | |
| | NEVTEFAKTCVADESAENCDKSLHTLFGDKLCTVATLRETYG | |
| | EMADCCAKQEPERNECFLQHKDDNPNLPRLVRPEVDVMCTA | |
| | FHDNEETFLKKYLYEIARRHPYFYAPELLFFAKRYKAAFTECC | |
| | QAADKAACLLPKLDELRDEGKASSAKQRLKCASLQKFGERA | |
| | FKAWAVARLSQRFPKAEFAEVSKLVTDLTKVHTECCHGDLL | |
| | ECADDRADLAKYICENQDSISSKLKECCEKPLLEKSHCIAEVE | |
| | NDEMPADLPSLAADFVESKDVCKNYAEAKDVFLGMFLYEYA | |
| | RRHPDYSVVLLLRLAKTYETTLEKCCAAADPHECYAKVFDEF | |
| | KPLVEEPQNLIKQNCELFEQLGEYKFQNALLVRYTKKVPQVS | |
| | TPTLVEVSRNLGKVGSKCCKHPEAKRMPCAEDYLSVVLNQL | |
| | CVLHEKTPVSDRVTKCCTESLVNRRPCFSALEVDETYVPKEF | |
| | NAETFTFHADICTLSEKERQIKKQTALVELVKHKPKATKEQLK | |
| | AVMDDFAAFVEKCCKADDKETCFAEEGKKLVAASQAALGL | |
| CBD-IL-35 | CSQPLDVILLLDGSSSFPASYFDEMKSFAKAFISKANIGPRLTQ | 54 |
| | VSVLQYGSITTIDVPWNVVPEKAHLLSLVDVMQREGGPSQIG | |
| | DALGFAVRYLTSEMHGARPGASKAVVILVTDVSVDSVDAAA | |
| | DAARSNRVTVFPIGIGDRYDAAQLRILAGPAGDSNVVKLQRIE | |
| | DLPTMVTLGNSFLHKLCSGFVRIGSRAGPPAALTLPRVQCRAS | |
| | RYPIAVDCSWTLPPAPNSTSPVSFIATYRLGMAARGHSWPCLQ | |
| | QTPTSTSCTITDVQLFSMAPYVLNVTAVHPWGSSSSFVPFITEH | |
| | IIKPDPPEGVRLSPLAERQLQVQWEPPGSWPFPEIFSLKYWIRY | |
| | KRQGAARFHRVGPIEATSFILRAVRPRARYYVQVAAQDLTDY | |
| | GELSDWSLPATATMSLGKGGGSGGGSGGGSGGGSRNLPVAT | |
| | PDPGMFPCLHHSQNLLRAVSNMLQKARQTLEFYPCTSEEIDH | |
| | EDITKDKTSTVEACLPLELTKNESSLNSRETSFITNGSCLASRK | |
| | TSFMMALCLSSIYEDLKMYQVEFKTMNAKLLMDPKRQIFLD | |
| | QNMLAVIDELMQALNFNSETVPQKSSLEEPDFYKTKIKLCILL | |
| | HAFRIRAVTIDRVMSYLNAS | |
| IL-35-CBD | RAGPPAALTLPRVQCRASRYPIAVDCSWTLPPAPNSTSPVSFIA | 55 |
| | TYRLGMAARGHSWPCLQQTPTSTSCTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESS | |
| | LNSRETSFITNGSCLASRKTSFMMALCLSSIYEDLKMYQVEFK | |
| | TMNAKLLMDPKRQIFLDQNMLAVIDELMQALNFNSETVPQK | |
| | SSLEEPDFYKTKIKLCILLHAFRIRAVTIDRVMSYLNASCSQPL | |
| | DVILLLDGSSSFPASYFDEMKSFAKAFISKANIGPRLTQVSVLQ | |
| | YGSITTIDVPWNVVPEKAHLLSLVDVMQREGGPSQIGDALGF | |
| | AVRYLTSEMHGARPGASKAVVILVTDVSVDSVDAAADAARS | |
| | NRVTVFPIGIGDRYDAAQLRILAGPAGDSNVVKLQRIEDLPTM | |
| | VTLGNSFLHKLCSGFVRIGS | |
| HSA-Ebi3- | DAHKSEVAHRFKDLGEENFKALVLIAFAQYLQQAPFEDHVKL | 56 |
| p35 | VNEVTEFAKTCVADESAENCDKSLHTLFGDKLCTVATLRETY | |
| | GEMADCCAKQEPERNECFLQHKDDNPNLPRLVRPEVDVMCT | |
| | AFHDNEETFLKKYLYEIARRHPYFYAPELLFFAKRYKAAFTEC | |
| | CQAADKAACLLPKLDELRDEGKASSAKQRLKCASLQKFGER | |
| | AFKAWAVARLSQRFPKAEFAEVSKLVTDLTKVHTECCHGDL | |
| | LECADDRADLAKYICENQDSISSKLKECCEKPLLEKSHCIAEV | |
| | ENDEMPADLPSLAADFVESKDVCKNYAEAKDVFLGMFLYEY | |
| | ARRHPDYSVVLLLRLAKTYETTLEKCCAAADPHECYAKVFD | |
| | EFKPLVEEPQNLIKQNCELFEQLGEYKFQNALLVRYTKKVPQ | |
| | VSTPTLVEVSRNLGKVGSKCCKHPEAKRMPCAEDYLSVVLN | |
| | QLCVLHEKTPVSDRVTKCCTESLVNRRPCFSALEVDETYVPK | |
| | EFNAETFTFHADICTLSEKERQIKKQTALVELVKHKPKATKEQ | |
| | LKAVMDDFAAFVEKCCKADDKETCFAEEGKKLVAASQAAL | |
| | GLGGGSGGGSGGGSGGGSGGGSRAGPPAALTLPRVQCRASR | |
| | YPIAVDCSWTLPPAPNSTSPVSFIATYRLGMAARGHSWPCLQ | |
| | QTPTSTSCTITDVQLFSMAPYVLNVTAVHPWGSSSSFVPFITEH | |
| | IIKPDPPEGVRLSPLAERQLQVQWEPPGSWPFPEIFSLKYWIRY | |
| | KRQGAARFHRVGPIEATSFILRAVRPRARYYVQVAAQDLTDY | |
| | GELSDWSLPATATMSLGKGGGSGGGSGGGSGGGSRNLPVAT | |
| | PDPGMFPCLHHSQNLLRAVSNMLQKARQTLEFYPCTSEEIDH | |
| | EDITKDKTSTVEACLPLELTKNESSLNSRETSFITNGSCLASRK | |
| | TSFMMALCLSSIYEDLKMYQVEFKTMNAKLLMDPKRQIFLD | |
| | QNMLAVIDELMQALNFNSETVPQKSSLEEPDFYKTKIKLCILL | |
| | HAFRIRAVTIDRVMSYLNA | |
| HSA-p35- | DAHKSEVAHRFKDLGEENFKALVLIAFAQYLQQAPFEDHVKL | 57 |
| Ebi3 | VNEVTEFAKTCVADESAENCDKSLHTLFGDKLCTVATLRETY | |
| | GEMADCCAKQEPERNECFLQHKDDNPNLPRLVRPEVDVMCT | |
| | AFHDNEETFLKKYLYEIARRHPYFYAPELLFFAKRYKAAFTEC | |
| | CQAADKAACLLPKLDELRDEGKASSAKQRLKCASLQKFGER | |
| | AFKAWAVARLSQRFPKAEFAEVSKLVTDLTKVHTECCHGDL | |
| | LECADDRADLAKYICENQDSISSKLKECCEKPLLEKSHCIAEV | |
| | ENDEMPADLPSLAADFVESKDVCKNYAEAKDVFLGMFLYEY | |
| | ARRHPDYSVVLLLRLAKTYETTLEKCCAAADPHECYAKVFD | |
| | EFKPLVEEPQNLIKQNCELFEQLGEYKFQNALLVRYTKKVPQ | |
| | VSTPTLVEVSRNLGKVGSKCCKHPEAKRMPCAEDYLSVVLN | |
| | QLCVLHEKTPVSDRVTKCCTESLVNRRPCFSALEVDETYVPK | |
| | EFNAETFTFHADICTLSEKERQIKKQTALVELVKHKPKATKEQ | |
| | LKAVMDDFAAFVEKCCKADDKETCFAEEGKKLVAASQAAL | |
| | GLGGGSGGGSGGGSGGGSGGGSRNLPVATPDPGMFPCLHHS | |
| | QNLLRAVSNMLQKARQTLEFYPCTSEEIDHEDITKDKTSTVEA | |
| | CLPLELTKNESSLNSRETSFITNGSCLASRKTSFMMALCLSSIY | |
| | EDLKMYQVEFKTMNAKLLMDPKRQIFLDQNMLAVIDELMQ | |
| | ALNFNSETVPQKSSLEEPDFYKTKIKLCILLHAFRIRAVTIDRV | |
| | MSYLNAGGGSGGGSGGGSGGGSRAGPPAALTLPRVQCRASR | |
| | YPIAVDCSWTLPPAPNSTSPVSFIATYRLGMAARGHSWPCLQ | |
| | QTPTSTSCTITDVQLFSMAPYVLNVTAVHPWGSSSSFVPFITEH | |
| | IIKPDPPEGVRLSPLAERQLQVQWEPPGSWPFPEIFSLKYWIRY | |
| | KRQGAARFHRVGPIEATSFILRAVRPRARYYVQVAAQDLTDY | |
| | GELSDWSLPATATMSLGK | |
| p35-Ebi3 | RNLPVATPDPGMFPCLHHSQNLLRAVSNMLQKARQTLEFYPC | 58 |
| | TSEEIDHEDITKDKTSTVEACLPLELTKNESSLNSRETSFITNGS | |
| | CLASRKTSFMMALCLSSIYEDLKMYQVEFKTMNAKLLMDPK | |
| | RQIFLDQNMLAVIDELMQALNFNSETVPQKSSLEEPDFYKTKI | |
| | KLCILLHAFRIRAVTIDRVMSYLNAGGGSGGGSGGGSGGGSR | |
| | AGPPAALTLPRVQCRASRYPIAVDCSWTLPPAPNSTSPVSFIAT | |
| | YRLGMAARGHSWPCLQQTPTSTSCTITDVQLFSMAPYVLNVT | |
| | AVHPWGSSSSFVPFITEHIIKPDPPEGVRLSPLAERQLQVQWEP | |
| | PGSWPFPEIFSLKYWIRYKRQGAARFHRVGPIEATSFILRAVRP | |
| | RARYYVQVAAQDLTDYGELSDWSLPATATMSLGK | |
| p35-Ebi3- | RNLPVATPDPGMFPCLHHSQNLLRAVSNMLQKARQTLEFYPC | 59 |
| Fc | TSEEIDHEDITKDKTSTVEACLPLELTKNESSLNSRETSFITNGS | |
| | CLASRKTSFMMALCLSSIYEDLKMYQVEFKTMNAKLLMDPK | |
| | RQIFLDQNMLAVIDELMQALNENSETVPQKSSLEEPDFYKTKI | |
| | KLCILLHAFRIRAVTIDRVMSYLNAGGGSGGGSGGGSGGGSR | |
| | AGPPAALTLPRVQCRASRYPIAVDCSWTLPPAPNSTSPVSFIAT | |
| | YRLGMAARGHSWPCLQQTPTSTSCTITDVQLFSMAPYVLNVT | |
| | AVHPWGSSSSFVPFITEHIIKPDPPEGVRLSPLAERQLQVQWEP | |
| | PGSWPFPEIFSLKYWIRYKRQGAARFHRVGPIEATSFILRAVRP | |
| | RARYYVQVAAQDLTDYGELSDWSLPATATMSLGKEPKSCDK | |
| | THTCPPCPAPEFEGGPSVFLFPPKPKDTLMISRTPEVTCVVVDV | |
| | SHEDPEVKFNWYVDGVEVHNAKTKPREEQYASTYRVVSVLT | |
| | VLHQDWLNGKEYKCKVSNKALPASIEKTISKAKGQPREPQVY | |
| | TLPPSRDELTKNQVSLTCLVKGFYPSDIAVEWESNGQPENNY | |
| | KTTPPVLDSDGSFFLYSKLTVDKSRWQQGNVFSCSVLHEALH | |
| | SHYTQKSLSLSPGK | |
| IL35- | RAGPPAALTLPRVQCRASRYPIAVDCSWTLPPAPNSTSPVSFIA | 60 |
| albumin | TYRLGMAARGHSWPCLQQTPTSTSCTITDVQLFSMAPYVLNV | |
| binding | TAVHPWGSSSSFVPFITEHIIKPDPPEGVRLSPLAERQLQVQWE | |
| peptide | PPGSWPFPEIFSLKYWIRYKRQGAARFHRVGPIEATSFILRAVR | |
| | PRARYYVQVAAQDLTDYGELSDWSLPATATMSLGKRNLPVA | |
| | TPDPGGGSGGGSGGGSGGGSRNLPVATPDPGMFPCLHHSQNL | |
| | LRAVSNMLQKARQTLEFYPCTSEEIDHEDITKDKTSTVEACLP | |
| | LELTKNESSLNSRETSFITNGSCLASRKTSFMMALCLSSIYEDL | |
| | KMYQVEFKTMNAKLLMDPKRQIFLDQNMLAVIDELMQALNF | |
| | NSETVPQKSSLEEPDFYKTKIKLCILLHAFRIRAVTIDRVMSYL | |
| | NASDICLPRWGCLW | |
| Delta20 | PIAVDCSWTLPPAPNSTSPVSFIATYRLGMAARGHSWPCLQQT | 61 |
| IL35- | PTSTSCTITDVQLESMAPYVLNVTAVHPWGSSSSFVPFITEHIIK | |
| albumin | PDPPEGVRLSPLAERQLQVQWEPPGSWPFPEIFSLKYWIRYKR | |
| binding | QGAARFHRVGPIEATSFILRAVRPRARYYVQVAAQDLTDYGE | |
| peptide | LSDWSLPATATMSLGKRNLPVATPDPGGGSGGGSGGGSGGG | |
| | SRNLPVATPDPGMFPCLHHSQNLLRAVSNMLQKARQTLEFYP | |
| | CTSEEIDHEDITKDKTSTVEACLPLELTKNESSLNSRETSFITNG | |
| | SCLASRKTSFMMALCLSSIYEDLKMYQVEFKTMNAKLLMDP | |
| | KRQIFLDQNMLAVIDELMQALNFNSETVPQKSSLEEPDFYKT | |
| | KIKLCILLHAFRIRAVTIDRVMSYLNASDICLPRWGCLW | |
| Delta20 | PIAVDCSWTLPPAPNSTSPVSFIATYRLGMAARGHSWPSLQQT | 62 |
| Ser1 IL35- | PTSTSCTITDVQLFSMAPYVLNVTAVHPWGSSSSFVPFITEHIIK | |
| albumin | PDPPEGVRLSPLAERQLQVQWEPPGSWPFPEIFSLKYWIRYKR | |
| binding | QGAARFHRVGPIEATSFILRAVRPRARYYVQVAAQDLTDYGE | |
| peptide | LSDWSLPATATMSLGKRNLPVATPDPGGGSGGGSGGGSGGG | |
| | SRNLPVATPDPGMFPCLHHSQNLLRAVSNMLQKARQTLEFYP | |
| | CTSEEIDHEDITKDKTSTVEACLPLELTKNESSLNSRETSFITNG | |
| | SCLASRKTSFMMALCLSSIYEDLKMYQVEFKTMNAKLLMDP | |
| | KRQIFLDQNMLAVIDELMQALNFNSETVPQKSSLEEPDFYKT | |
| | KIKLCILLHAFRIRAVTIDRVMSYLNASDICLPRWGCLW | |
| Delta20 | PIAVDSSWTLPPAPNSTSPVSFIATYRLGMAARGHSWPSLQQT | 63 |
| Ser2 IL35- | PTSTSCTITDVQLESMAPYVLNVTAVHPWGSSSSFVPFITEHIIK | |
| albumin | PDPPEGVRLSPLAERQLQVQWEPPGSWPFPEIFSLKYWIRYKR | |
| binding | QGAARFHRVGPIEATSFILRAVRPRARYYVQVAAQDLTDYGE | |
| peptide | LSDWSLPATATMSLGKRNLPVATPDPGGGSGGGSGGGSGGG | |
| | SRNLPVATPDPGMFPCLHHSQNLLRAVSNMLQKARQTLEFYP | |
| | CTSEEIDHEDITKDKTSTVEACLPLELTKNESSLNSRETSFITNG | |
| | SCLASRKTSFMMALCLSSIYEDLKMYQVEFKTMNAKLLMDP | |
| | KRQIFLDQNMLAVIDELMQALNFNSETVPQKSSLEEPDFYKT | |
| | KIKLCILLHAFRIRAVTIDRVMSYLNASDICLPRWGCLW | |
| Delta20 | PIAVDSSWTLPPAPNSTSPVSFIATYRLGMAARGHSWPSLQQT | 64 |
| Ser3 IL35- | PTSTSSTITDVQLFSMAPYVLNVTAVHPWGSSSSFVPFITEHIIK | |
| albumin | PDPPEGVRLSPLAERQLQVQWEPPGSWPFPEIFSLKYWIRYKR | |
| binding | QGAARFHRVGPIEATSFILRAVRPRARYYVQVAAQDLTDYGE | |
| peptide | LSDWSLPATATMSLGKRNLPVATPDPGGGSGGGSGGGSGGG | |
| | SGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNADICLPRWGCLW | |
| IL-35- | RAGPPAALTLPRVQCRASRYPIAVDCSWTLPPAPNSTSPVSFIA | 65 |
| GGGG- | TYRLGMAARGHSWPCLQQTPTSTSCTITDVQLFSMAPYVLNV | |
| HSA | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PRARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESS | |
| | LNSRETSFITNGSCLASRKTSFMMALCLSSIYEDLKMYQVEFK | |
| | TMNAKLLMDPKRQIFLDQNMLAVIDELMQALNENSETVPQK | |
| | SSLEEPDFYKTKIKLCILLHAFRIRAVTIDRVMSYLNASGGGG | |
| | DAHKSEVAHRFKDLGEENFKALVLIAFAQYLQQAPFEDHVKL | |
| | VNEVTEFAKTCVADESAENCDKSLHTLFGDKLCTVATLRETY | |
| | GEMADCCAKQEPERNECFLQHKDDNPNLPRLVRPEVDVMCT | |
| | AFHDNEETFLKKYLYEIARRHPYFYAPELLFFAKRYKAAFTEC | |
| | CQAADKAACLLPKLDELRDEGKASSAKQRLKCASLQKFGER | |
| | AFKAWAVARLSQRFPKAEFAEVSKLVTDLTKVHTECCHGDL | |
| | LECADDRADLAKYICENQDSISSKLKECCEKPLLEKSHCIAEV | |
| | ENDEMPADLPSLAADFVESKDVCKNYAEAKDVFLGMFLYEY | |
| | ARRHPDYSVVLLLRLAKTYETTLEKCCAAADPHECYAKVFD | |
| | EFKPLVEEPQNLIKQNCELFEQLGEYKFQNALLVRYTKKVPQ | |
| | VSTPTLVEVSRNLGKVGSKCCKHPEAKRMPCAEDYLSVVLN | |
| | QLCVLHEKTPVSDRVTKCCTESLVNRRPCFSALEVDETYVPK | |
| | EFNAETFTFHADICTLSEKERQIKKQTALVELVKHKPKATKEQ | |
| | LKAVMDDFAAFVEKCCKADDKETCFAEEGKKLVAASQAAL | |
| | GL | |
| delta20 IL- | PIAVDCSWTLPPAPNSTSPVSFIATYRLGMAARGHSWPCLQQT | 66 |
| 35-GGGG- | PTSTSCTITDVQLFSMAPYVLNVTAVHPWGSSSSFVPFITEHIIK | |
| HSA | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASGGGGDAHKSEVAHRFKDLGEENFKAL | |
| | VLIAFAQYLQQAPFEDHVKLVNEVTEFAKTCVADESAENCDK | |
| | SLHTLFGDKLCTVATLRETYGEMADCCAKQEPERNECFLQHK | |
| | DDNPNLPRLVRPEVDVMCTAFHDNEETFLKKYLYEIARRHPY | |
| | FYAPELLFFAKRYKAAFTECCQAADKAACLLPKLDELRDEGK | |
| | ASSAKQRLKCASLQKFGERAFKAWAVARLSQRFPKAEFAEVS | |
| | KLVTDLTKVHTECCHGDLLECADDRADLAKYICENQDSISSK | |
| | LKECCEKPLLEKSHCIAEVENDEMPADLPSLAADFVESKDVC | |
| | KNYAEAKDVFLGMFLYEYARRHPDYSVVLLLRLAKTYETTL | |
| | EKCCAAADPHECYAKVFDEFKPLVEEPQNLIKQNCELFEQLG | |
| | EYKFQNALLVRYTKKVPQVSTPTLVEVSRNLGKVGSKCCKHP | |
| | EAKRMPCAEDYLSVVLNQLCVLHEKTPVSDRVTKCCTESLV | |
| | NRRPCFSALEVDETYVPKEFNAETFTFHADICTLSEKERQIKK | |
| | QTALVELVKHKPKATKEQLKAVMDDFAAFVEKCCKADDKE | |
| | TCFAEEGKKLVAASQAALGL | |
| delta20 | PIAVDCSWTLPPAPNSTSPVSFIATYRLGMAARGHSWPSLQQT | 67 |
| Ser1 IL-35- | PTSTSCTITDVQLFSMAPYVLNVTAVHPWGSSSSFVPFITEHIIK | |
| GGGG- | PDPPEGVRLSPLAERQLQVQWEPPGSWPFPEIFSLKYWIRYKR | |
| HSA | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASGGGGDAHKSEVAHRFKDLGEENFKAL | |
| | VLIAFAQYLQQAPFEDHVKLVNEVTEFAKTCVADESAENCDK | |
| | SLHTLFGDKLCTVATLRETYGEMADCCAKQEPERNECFLQHK | |
| | DDNPNLPRLVRPEVDVMCTAFHDNEETFLKKYLYEIARRHPY | |
| | FYAPELLFFAKRYKAAFTECCQAADKAACLLPKLDELRDEGK | |
| | ASSAKQRLKCASLQKFGERAFKAWAVARLSQRFPKAEFAEVS | |
| | KLVTDLTKVHTECCHGDLLECADDRADLAKYICENQDSISSK | |
| | LKECCEKPLLEKSHCIAEVENDEMPADLPSLAADFVESKDVC | |
| | KNYAEAKDVFLGMFLYEYARRHPDYSVVLLLRLAKTYETTL | |
| | EKCCAAADPHECYAKVFDEFKPLVEEPQNLIKQNCELFEQLG | |
| | EYKFQNALLVRYTKKVPQVSTPTLVEVSRNLGKVGSKCCKHP | |
| | EAKRMPCAEDYLSVVLNQLCVLHEKTPVSDRVTKCCTESLV | |
| | NRRPCFSALEVDETYVPKEFNAETFTFHADICTLSEKERQIKK | |
| | QTALVELVKHKPKATKEQLKAVMDDFAAFVEKCCKADDKE | |
| | TCFAEEGKKLVAASQAALGL | |
| delta20 IL- | PIAVDSSWTLPPAPNSTSPVSFIATYRLGMAARGHSWPSLQQT | 68 |
| 35-GGGG- | PTSTSCTITDVQLESMAPYVLNVTAVHPWGSSSSFVPFITEHIIK | |
| HSA | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASGGGGDAHKSEVAHRFKDLGEENFKAL | |
| | VLIAFAQYLQQAPFEDHVKLVNEVTEFAKTCVADESAENCDK | |
| | SLHTLFGDKLCTVATLRETYGEMADCCAKQEPERNECFLQHK | |
| | DDNPNLPRLVRPEVDVMCTAFHDNEETFLKKYLYEIARRHPY | |
| | FYAPELLFFAKRYKAAFTECCQAADKAACLLPKLDELRDEGK | |
| | ASSAKQRLKCASLQKFGERAFKAWAVARLSQRFPKAEFAEVS | |
| | KLVTDLTKVHTECCHGDLLECADDRADLAKYICENQDSISSK | |
| | LKECCEKPLLEKSHCIAEVENDEMPADLPSLAADFVESKDVC | |
| | KNYAEAKDVFLGMFLYEYARRHPDYSVVLLLRLAKTYETTL | |
| | EKCCAAADPHECYAKVFDEFKPLVEEPQNLIKQNCELFEQLG | |
| | EYKFQNALLVRYTKKVPQVSTPTLVEVSRNLGKVGSKCCKHP | |
| | EAKRMPCAEDYLSVVLNQLCVLHEKTPVSDRVTKCCTESLV | |
| | NRRPCFSALEVDETYVPKEFNAETFTFHADICTLSEKERQIKK | |
| | QTALVELVKHKPKATKEQLKAVMDDFAAFVEKCCKADDKE | |
| | TCFAEEGKKLVAASQAALGL | |
| delta20 | PIAVDSSWTLPPAPNSTSPVSFIATYRLGMAARGHSWPSLQQT | 69 |
| Ser3 IL-35- | PTSTSSTITDVQLFSMAPYVLNVTAVHPWGSSSSFVPFITEHIIK | |
| GGGG- | PDPPEGVRLSPLAERQLQVQWEPPGSWPFPEIFSLKYWIRYKR | |
| HSA | QGAARFHRVGPIEATSFILRAVRPRARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESSLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASGGGGDAHKSEVAHRFKDLGEENFKAL | |
| | VLIAFAQYLQQAPFEDHVKLVNEVTEFAKTCVADESAENCDK | |
| | SLHTLFGDKLCTVATLRETYGEMADCCAKQEPERNECFLQHK | |
| | DDNPNLPRLVRPEVDVMCTAFHDNEETFLKKYLYEIARRHPY | |
| | FYAPELLFFAKRYKAAFTECCQAADKAACLLPKLDELRDEGK | |
| | ASSAKQRLKCASLQKFGERAFKAWAVARLSQRFPKAEFAEVS | |
| | KLVTDLTKVHTECCHGDLLECADDRADLAKYICENQDSISSK | |
| | LKECCEKPLLEKSHCIAEVENDEMPADLPSLAADFVESKDVC | |
| | KNYAEAKDVFLGMFLYEYARRHPDYSVVLLLRLAKTYETTL | |
| | EKCCAAADPHECYAKVFDEFKPLVEEPQNLIKQNCELFEQLG | |
| | EYKFQNALLVRYTKKVPQVSTPTLVEVSRNLGKVGSKCCKHP | |
| | EAKRMPCAEDYLSVVLNQLCVLHEKTPVSDRVTKCCTESLV | |
| | NRRPCFSALEVDETYVPKEFNAETFTFHADICTLSEKERQIKK | |
| | QTALVELVKHKPKATKEQLKAVMDDFAAFVEKCCKADDKE | |
| | TCFAEEGKKLVAASQAALGL | |
| IL-35-Fc2 | RAGPPAALTLPRVQCRASRYPIAVDCSWTLPPAPNSTSPVSFIA | 70 |
| | TYRLGMAARGHSWPCLQQTPTSTSCTITDVQLFSMAPYVLNV | |
| | TAVHPWGSSSSFVPFITEHIIKPDPPEGVRLSPLAERQLQVQWE | |
| | PPGSWPFPEIFSLKYWIRYKRQGAARFHRVGPIEATSFILRAVR | |
| | PAARYYVQVAAQDLTDYGELSDWSLPATATMSLGKGGGSG | |
| | GGSGGGSGGGSRNLPVATPDPGMFPCLHHSQNLLRAVSNML | |
| | QKARQTLEFYPCTSEEIDHEDITKDKTSTVEACLPLELTKNESC | |
| | LNSRETSFITNGSCLASRKTSFMMALCLSSIYEDLKMYQVEFK | |
| | TMNAKLLMDPKRQIFLDQNMLAVIDELMQALNFNSETVPQK | |
| | SSLEEPDFYKTKIKLCILLHAFRIRAVTIDRVMSYLNASGGGSG | |
| | GGSDKTHTCPPCPAPEFEGGPSVFLFPPKPKDTLMISRTPEVTC | |
| | VVVDVSHEDPEVKFNWYVDGVEVHNAKTKPREEQYASTYR | |
| | VVSVLTVLHQDWLNGKEYKCKVSNKALPASIEKTISKAKGQP | |
| | REPQVYTLPPSREEMTKNQVSLTCLVKGFYPSDIAVEWESNG | |
| | QPENNYKTTPPVLDSDGSFFLYSKLTVDKSRWQQGNVFSCSV | |
| | MHEALHNHYTQKSLSLSPGK | |
| Delta20 IL- | PIAVDCSWTLPPAPNSTSPVSFIATYRLGMAARGHSWPCLQQT | 71 |
| 35-Fc2 | PTSTSCTITDVQLFSMAPYVLNVTAVHPWGSSSSFVPFITEHIIK | |
| | PDPPEGVRLSPLAERQLQVQWEPPGSWPFPEIFSLKYWIRYKR | |
| | QGAARFHRVGPIEATSFILRAVRPAARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESCLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASGGGSGGGSDKTHTCPPCPAPEFEGGPS | |
| | VFLFPPKPKDTLMISRTPEVTCVVVDVSHEDPEVKFNWYVDG | |
| | VEVHNAKTKPREEQYASTYRVVSVLTVLHQDWLNGKEYKC | |
| | KVSNKALPASIEKTISKAKGQPREPQVYTLPPSREEMTKNQVS | |
| | LTCLVKGFYPSDIAVEWESNGQPENNYKTTPPVLDSDGSFFLY | |
| | SKLTVDKSRWQQGNVFSCSVMHEALHNHYTQKSLSLSPGK | |
| Delta20 | PIAVDCSWTLPPAPNSTSPVSFIATYRLGMAARGHSWPSLQQT | 72 |
| Ser1 IL-35- | PTSTSCTITDVQLFSMAPYVLNVTAVHPWGSSSSFVPFITEHIIK | |
| Fc2 | PDPPEGVRLSPLAERQLQVQWEPPGSWPFPEIFSLKYWIRYKR | |
| | QGAARFHRVGPIEATSFILRAVRPAARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESCLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASGGGSGGGSDKTHTCPPCPAPEFEGGPS | |
| | VFLFPPKPKDTLMISRTPEVTCVVVDVSHEDPEVKFNWYVDG | |
| | VEVHNAKTKPREEQYASTYRVVSVLTVLHQDWLNGKEYKC | |
| | KVSNKALPASIEKTISKAKGQPREPQVYTLPPSREEMTKNQVS | |
| | LTCLVKGFYPSDIAVEWESNGQPENNYKTTPPVLDSDGSFFLY | |
| | SKLTVDKSRWQQGNVFSCSVMHEALHNHYTQKSLSLSPGK | |
| Delta20 | PIAVDSSWTLPPAPNSTSPVSFIATYRLGMAARGHSWPSLQQT | 73 |
| Ser2 IL-35- | PTSTSCTITDVQLFSMAPYVLNVTAVHPWGSSSSFVPFITEHIIK | |
| Fc2 | PDPPEGVRLSPLAERQLQVQWEPPGSWPFPEIFSLKYWIRYKR | |
| | QGAARFHRVGPIEATSFILRAVRPAARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESCLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASGGGSGGGSDKTHTCPPCPAPEFEGGPS | |
| | VFLFPPKPKDTLMISRTPEVTCVVVDVSHEDPEVKFNWYVDG | |
| | VEVHNAKTKPREEQYASTYRVVSVLTVLHQDWLNGKEYKC | |
| | KVSNKALPASIEKTISKAKGQPREPQVYTLPPSREEMTKNQVS | |
| | LTCLVKGFYPSDIAVEWESNGQPENNYKTTPPVLDSDGSFFLY | |
| | SKLTVDKSRWQQGNVFSCSVMHEALHNHYTQKSLSLSPGK | |
| Delta20 | PIAVDSSWTLPPAPNSTSPVSFIATYRLGMAARGHSWPSLQQT | 74 |
| Ser3 IL-35- | PTSTSSTITDVQLFSMAPYVLNVTAVHPWGSSSSFVPFITEHIIK | |
| Fc2 | PDPPEGVRLSPLAERQLQVQWEPPGSWPFPEIFSLKYWIRYKR | |
| | QGAARFHRVGPIEATSFILRAVRPAARYYVQVAAQDLTDYGE | |
| | LSDWSLPATATMSLGKGGGSGGGSGGGSGGGSRNLPVATPD | |
| | PGMFPCLHHSQNLLRAVSNMLQKARQTLEFYPCTSEEIDHEDI | |
| | TKDKTSTVEACLPLELTKNESCLNSRETSFITNGSCLASRKTSF | |
| | MMALCLSSIYEDLKMYQVEFKTMNAKLLMDPKRQIFLDQNM | |
| | LAVIDELMQALNFNSETVPQKSSLEEPDFYKTKIKLCILLHAFR | |
| | IRAVTIDRVMSYLNASGGGSGGGSDKTHTCPPCPAPEFEGGPS | |
| | VFLFPPKPKDTLMISRTPEVTCVVVDVSHEDPEVKFNWYVDG | |
| | VEVHNAKTKPREEQYASTYRVVSVLTVLHQDWLNGKEYKC | |
| | KVSNKALPASIEKTISKAKGQPREPQVYTLPPSREEMTKNQVS | |
| | LTCLVKGFYPSDIAVEWESNGQPENNYKTTPPVLDSDGSFFLY | |
| | SKLTVDKSRWQQGNVFSCSVMHEALHNHYTQKSLSLSPGK | |
| Fc | EPKSCDKTHTCPPCPAPEFEGGPSVFLFPPKPKDTLMISRTPEV | 86 |
| | TCVVVDVSHEDPEVKFNWYVDGVEVHNAKTKPREEQYAST | |
| | YRVVSVLTVLHQDWLNGKEYKCKVSNKALPASIEKTISKAKG | |
| | QPREPQVYTLPPSRDELTKNQVSLTCLVKGFYPSDIAVEWESN | |
| | GQPENNYKTTPPVLDSDGSFFLYSKLTVDKSRWQQGNVFSCS | |
| | VLHEALHSHYTQKSLSLSPGK | |
| Fc2 | DKTHTCPPCPAPEFEGGPSVFLFPPKPKDTLMISRTPEVTCVV | 87 |
| | VDVSHEDPEVKFNWYVDGVEVHNAKTKPREEQYASTYRVVS | |
| | VLTVLHQDWLNGKEYKCKVSNKALPASIEKTISKAKGQPREP | |
| | QVYTLPPSREEMTKNQVSLTCLVKGFYPSDIAVEWESNGQPE | |
| | NNYKTTPPVLDSDGSFFLYSKLTVDKSRWQQGNVFSCSVMH | |
| | EALHNHYTQKSLSLSPGK | |
| Von | CSQPLDVILLLDGSSSFPASYFDEMKSFAKAFISKANIGPRLTQ | 88 |
| Willebrand | VSVLQYGSITTIDVPWNVVPEKAHLLSLVDVMQREGGPSQIG | |
| factor A3 | DALGFAVRYLTSEMHGARPGASKAVVILVTDVSVDSVDAAA | |
| domain | DAARSNRVTVFPIGIGDRYDAAQLRILAGPAGDSNVVKLQRIE | |
| (collagen | DLPTMVTLGNSFLHKLCSGFVRIGS | |
| binding domain: | | |
| CBD)-GS | | |
| Delta20 | PIAVDCSWTLPPAPNSTSPVSFIATYRLGMAARGHSWPCLQQT | 92 |
| EBI3 | PTSTSCTITDVQLFSMAPYVLNVTAVHPWGSSSSFVPFITEHIIK | |
| | PDPPEGVRLSPLAERQLQVQWEPPGSWPFPEIFSLKYWIRYKR | |
| | QGAARFHRVGPIEATSFILRAVRPRARYYVQVAAQDLTDYGE | |
| | LSDWSLPATATMSLGK | |
| Albumin | DICLPRWGCLW | 93 |
| Binding Protein | | |

### VIII. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

In the examples, figures, and figure descriptions, amino acids K22, R196, R73, and R194 refer to the amino acids at positions 23, 197, 74, and 194, respectively of the IL-35 amino acid sequence shown in SEQ ID NO:1 and the EBI3 subunit sequence shown in SEQ ID NO:2.

### Example 1: Secretion Assay of IL-35-HSA Mutants

The expression plasmids were constructed based on pcDNA 3.1 (Thermo Fisher Scientific, US). The cDNA fragment encoding the full length (SEQ ID NO:1) was synthesized by GeneArt Gene Synthesis Service (Thermo Fisher Scientific, US). To generate the expression plasmid pcDNA-IL35-HSA/WT, cDNA fragment, after digestion with NheI and EcoRV, was cloned into pcDNA 3.1 cut with the same restriction enzymes. To construct each expression plasmid of IL-35-HSA mutant, site-directed mutagenesis was performed using standard method of inverse PCR with the template of pcDNA-IL35-HSA/WT. Sequencing confirmed that the mutation was correctly introduced. The constructed mutants are shown in Table 1.

The expression plasmids were linearized with restriction enzyme ScaI and transfected into CHO cells according to the manual of Lipofectamine 2000 (Thermo Fisher Scientific, US). Twenty-four hours after transfection, antibiotic selection was performed with 500 μg/ml of G418 (Thermo Fisher Scientific, US) to construct the stable expression pools.

The constructed stable expression pools were seeded into a 125 mL flask at a seeding density of 2×10⁶ cells/mL and incubated for 72 hours. The culture media were centrifuged at 10,000g for 10 min and the supernatants were collected.

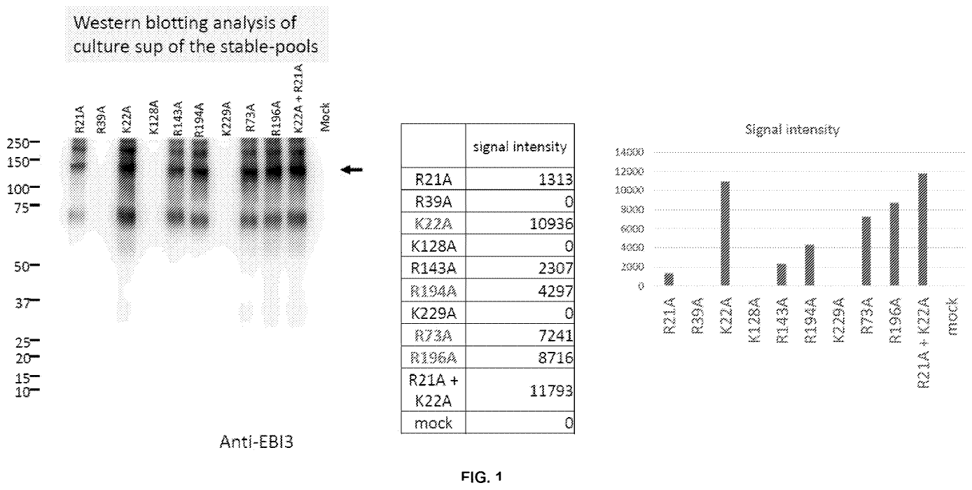

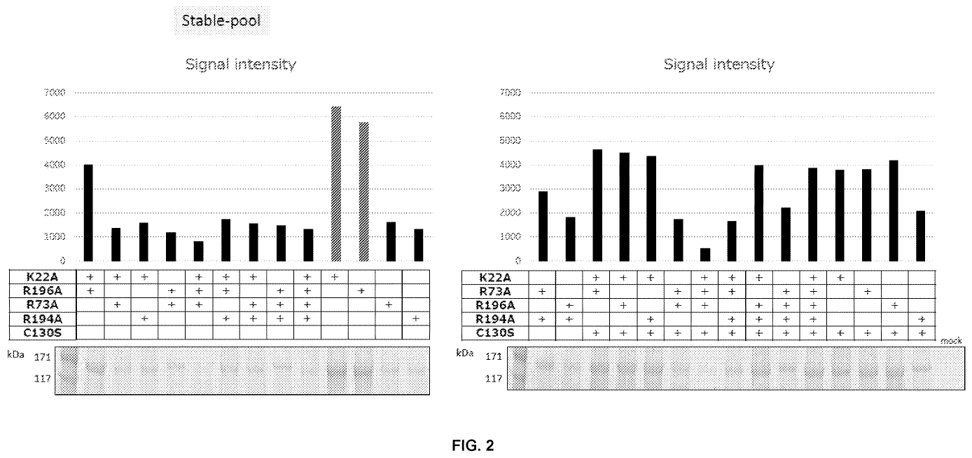

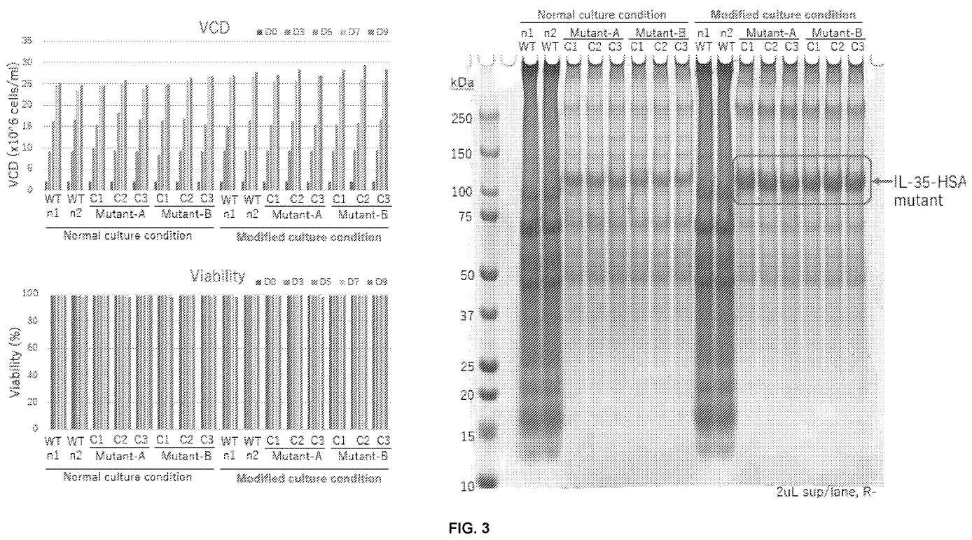

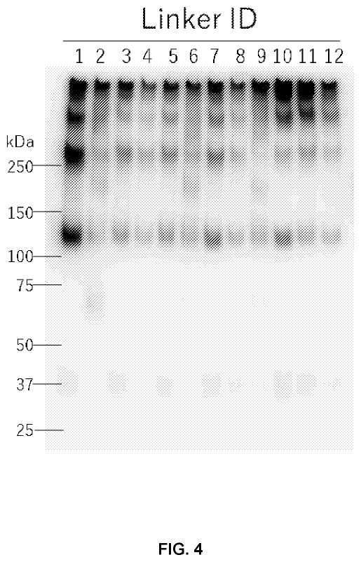

Each secretion levels of IL-35-HSA WT and IL-35-HSA mutants were then analyzed by SDS-PAGE and image analysis of the obtained signal intensities was performed with ImageJ software (found online at//imagej.nih.gov/ij/) to evaluate the amount of secreted proteins. The results showed increased secretion in the K22A and R196A mutants (Table 1, FIGS. 1-3).

TABLE 1

| Plasmid | | | | | | Secretion level, |
|---|---|---|---|---|---|---|
| ID | K22 | R73 | R194 | R196 | C320 | fold increase |
| 1 | — | — | — | — | — | 0.00 |
| 2 | A | — | — | — | — | 12.03 |
| 3 | — | A | — | — | — | 3.00 |
| 4 | — | — | A | — | — | 2.48 |
| 5 | — | — | — | A | — | 10.82 |
| 6 | A | A | — | — | — | 2.55 |
| 7 | A | — | A | — | — | 2.95 |
| 8 | A | — | — | A | — | 7.49 |
| 9 | A | — | — | — | A | 7.05 |
| 10 | — | A | A | — | — | 3.41 |
| 11 | — | A | — | A | — | 2.24 |
| 12 | — | A | — | — | A | 7.80 |
| 13 | — | — | A | A | — | 5.42 |
| 14 | — | — | A | — | A | 3.88 |
| 15 | — | — | — | A | A | 7.12 |
| 16 | A | A | A | — | — | 2.91 |
| 17 | A | A | — | A | — | 1.50 |
| 18 | A | A | — | — | A | 8.43 |
| 19 | A | — | A | A | — | 3.24 |
| 20 | A | — | A | — | A | 8.13 |
| 21 | A | — | — | A | A | 8.70 |
| 22 | — | A | A | A | — | 2.73 |
| 23 | | A | — | A | A | 3.26 |
| 24 | A | A | A | A | — | 2.51 |
| 25 | A | A | — | A | A | 1.00 |
| 26 | A | — | A | A | A | 3.06 |
| 27 | A | A | A | — | A | 7.43 |
| 28 | — | A | A | A | A | 4.18 |
| 29 | A | A | A | A | A | 7.21 |

### Example 2: Comprehensive Evaluation of IL-35-HSA, IL-35-Fc, and IL-35 Mutants in Secretion

The inventors made amino acid substitutions to all 19 different amino acids to compare IL-35 protein expressions.

The expression plasmids were constructed with 19 different amino acid substitutions for each of the 4 sites (K22, R196, R73, R194) of the EBI3 (Table 2) using the same method described in Example 1. The constructed mutants are shown in Table 2. The expression plasmids were transfected into CHO cells according to the manual of Lipofectamine 2000 (5×10⁴ cells/well, 96-well-plate, DNA 1 μg/well). The transfection method was followed the manual of Lipofectamine 2000. For IL-35, the p35/WT plasmid was also transfected simultaneously. Transfectants were incubated for 72 hours. The culture media were centrifuged at 10,000g for 10 min and the supernatants were collected. The amount of protein secreted into the culture supernatant was quantified by the Human EBI3 DuoSet ELISA (R&D SYSTEMS, P #: DY6456-05) (n=3). From the secreted protein concentration, the relative amount of secreted protein was calculated with the respective control as 1 (Table 3-5). Among the four site mutations, introduction of mutations to K22 and R196 markedly increased the secreted amount of IL-35-HSA, IL-35-Fc and IL-35. As for the introduction of mutations to R73 and P194, the effect on the secreted amount of IL-35-HSA, IL-35-Fc and IL-35 increased slightly. Among 19 amino acids, the most secreted amino acids were A, G>V, I>L>H, and S>T. Nonpolar amino acids were found to be preferred. The inventors also made and included different version of Fc as different blood half-life expected (SEQ ID NOs: 70-74)

TABLE 2

| Substituted | IL-35-HSA | | | | IL-35-Fc | | | | IL-35 | | | |
|---|---|---|---|---|---|---|---|---|---|---|---|---|
| amino acid | K22 | R196 | R73 | R194 | K22 | R196 | R73 | R194 | K22 | R196 | R73 | R194 |
| A | K22A | R196A | R73A | R194A | K22A | R196A | R73A | R194A | K22A | R196A | R73A | R194A |
| C | K22C | R196C | R73C | R194C | K22C | R196C | R73C | R194C | K22C | R196C | R73C | R194C |
| D | K22D | R196D | R73D | R194D | K22D | R196D | R73D | R194D | K22D | R196D | R73D | R194D |
| E | K22E | R196E | R73E | R194E | K22E | R196E | R73E | R194E | K22E | R196E | R73E | R194E |
| F | K22F | R196F | R73F | R194F | K22F | R196F | R73F | R194F | K22F | R196F | R73F | R194F |
| G | K22G | R196G | R73G | R194G | K22G | R196G | R73G | R194G | K22G | R196G | R73G | R194G |
| H | K22H | R196H | R73H | R194H | K22H | R196H | R73H | R194H | K22H | R196H | R73H | R194H |
| I | K22I | R196I | R73I | R194I | K22I | R196I | R73I | R194I | K22I | R196I | R73I | R194I |
| K | K22K | R196K | R73K | R194K | K22K | R196K | R73K | R194K | K22K | R196K | R73K | R194K |
| L | K22L | R196L | R73L | R194L | K22L | R196L | R73L | R194L | K22L | R196L | R73L | R194L |
| M | K22M | R196M | R73M | R194M | K22M | R196M | R73M | R194M | K22M | R196M | R73M | R194M |
| N | K22N | R196N | R73N | R194N | K22N | R196N | R73N | R194N | K22N | R196N | R73N | R194N |
| P | K22P | R196P | R73P | R194P | K22P | R196P | R73P | R194P | K22P | R196P | R73P | R194P |
| Q | K22Q | R196Q | R73Q | R194Q | K22Q | R196Q | R73Q | R194Q | K22Q | R196Q | R73Q | R194Q |
| R | K22R | R196R | R73R | R194R | K22R | R196R | R73R | R194R | K22R | R196R | R73R | R194R |
| S | K22S | R196S | R73S | R194S | K22S | R196S | R73S | R194S | K22S | R196S | R73S | R194S |
| T | K22T | R196T | R73T | R194T | K22T | R196T | R73T | R194T | K22T | R196T | R73T | R194T |
| V | K22V | R196V | R73V | R194V | K22V | R196V | R73V | R194V | K22V | R196V | R73V | R194V |
| W | K22W | R196W | R73W | R194W | K22W | R196W | R73W | R194W | K22W | R196W | R73W | R194W |
| Y | K22Y | R196Y | R73Y | R194Y | K22Y | R196Y | R73Y | R194Y | K22Y | R196Y | R73Y | R194Y |

TABLE 3
IL-35-HSA secretion (relative ratio, sorted)

| K22 | | R196 | | R73 | | R194 | |
|---|---|---|---|---|---|---|---|
| K22G | 10.6 | R196A | 9.9 | R73G | 2.9 | R194G | 2.6 |
| K22A | 10.5 | R196G | 9.7 | R73A | 2.9 | R194A | 2.4 |
| K22V | 8.9 | R196V | 8.2 | R73V | 2.9 | R194V | 2.3 |
| K22I | 8.9 | R196I | 7.9 | R73L | 2.6 | R194T | 2.1 |
| K22L | 8 | R196L | 7 | R73I | 2.5 | R194H | 2.1 |
| K22S | 6.3 | R196H | 5.5 | R73T | 2.1 | R194S | 2 |
| K22H | 6.2 | R196S | 5.5 | R73S | 1.9 | R194I | 1.9 |
| K22T | 5.6 | R196T | 4.5 | R73H | 1.8 | R194L | 1.9 |
| K22W | 2.9 | R196Y | 1.9 | R73F | 1.7 | R194F | 1.6 |
| K22Y | 2.8 | R196W | 1.9 | R73Y | 1.5 | R194Y | 1.6 |
| K22F | 2.7 | R196Q | 1.7 | R73Q | 1.5 | R194W | 1.5 |
| K22M | 2.6 | R196F | 1.6 | R73P | 1.5 | R194N | 1.5 |
| K22P | 2.2 | R196P | 1.6 | R73W | 1.4 | R194P | 1.4 |
| K22N | 2.1 | R196M | 1.6 | R73N | 1.4 | R194M | 1.3 |
| K22Q | 1.9 | R196N | 1.4 | R73D | 1.2 | R194Q | 1.2 |
| K22E | 1.4 | R196D | 1.3 | R73M | 1.2 | R194C | 1.2 |
| K22D | 1.2 | R196E | 1.3 | R73C | 1.2 | R194E | 1.2 |
| K22R | 1.2 | R196C | 1.2 | R73K | 1.1 | R194D | 1.1 |
| K22C | 1.2 | R196K | 1.2 | R73R | 1 | R194K | 1.1 |
| K22K | 1 | R196R | 1 | R73E | 0.9 | R194R | 1 |

TABLE 4
IL-35-Fc secretion (relative ratio, sorted)

| K22 | | R196 | | R73 | | R194 | |
|---|---|---|---|---|---|---|---|
| K22A | 9.5 | R196G | 9.2 | R73V | 2.8 | R194G | 2.2 |
| K22G | 9.4 | R196A | 9 | R73G | 2.7 | R194A | 2.2 |
| K22I | 7.8 | R196V | 7.7 | R73A | 2.5 | R194V | 2.1 |
| K22V | 7.8 | R196I | 7.5 | R73I | 2.2 | R194H | 2.1 |
| K22L | 7.3 | R196L | 6.6 | R73L | 2.2 | R194S | 2.1 |
| K22H | 5.8 | R196H | 5.1 | R73T | 2 | R194T | 1.8 |
| K22S | 5.7 | R196S | 5 | R73H | 1.9 | R194I | 1.8 |
| K22T | 4.7 | R196T | 4.3 | R73S | 1.9 | R194L | 1.8 |
| K22F | 2.6 | R196W | 2 | R73Y | 1.8 | R194Y | 1.5 |
| K22W | 2.5 | R196Y | 2 | R73F | 1.7 | R194W | 1.4 |
| K22Y | 2.4 | R196F | 2 | R73W | 1.5 | R194Q | 1.3 |
| K22M | 2.2 | R196P | 1.6 | R73C | 1.3 | R194F | 1.3 |
| K22P | 2 | R196N | 1.5 | R73N | 1.3 | R194E | 1.2 |
| K22Q | 1.9 | R196K | 1.4 | R73K | 1.2 | R194C | 1.1 |
| K22N | 1.6 | R196Q | 1.4 | R73M | 1.2 | R194P | 1 |
| K22E | 1.3 | R196M | 1.3 | R73Q | 1.2 | R194R | 1 |
| K22R | 1.2 | R196D | 1.1 | R73P | 1.2 | R194K | 1 |
| K22D | 1.1 | R196C | 1.1 | R73R | 1 | R194M | 1 |
| K22C | 1.1 | R196E | 1 | R73E | 0.9 | R194D | 0.9 |
| K22K | 1 | R196R | 1 | R73D | 0.9 | R194N | 0.8 |

TABLE 5
IL-35 secretion (relative ratio, sorted)

| K22 | | R196 | | R73 | | R194 | |
|---|---|---|---|---|---|---|---|
| K22G | 10.7 | R196G | 10.1 | R73G | 3.1 | R194G | 2.6 |
| K22A | 10.4 | R196A | 9.9 | R73V | 3.1 | R194V | 2.5 |
| K22I | 8.7 | R196V | 8.2 | R73A | 3 | R194A | 2.2 |
| K22V | 8.7 | R196I | 8 | R73I | 2.6 | R194S | 2.2 |
| K22L | 8 | R196L | 7.2 | R73L | 2.5 | R194I | 2.1 |
| K22H | 6.4 | R196S | 5.7 | R73S | 2.2 | R194T | 2.1 |
| K22S | 6.1 | R196H | 5.3 | R73H | 2.2 | R194L | 1.9 |
| K22T | 5.5 | R196T | 4.4 | R73T | 2.1 | R194H | 1.9 |
| K22Y | 3.2 | R196W | 2.1 | R73F | 1.7 | R194W | 1.6 |
| K22F | 3 | R196Y | 2.1 | R73W | 1.6 | R194Y | 1.6 |
| K22W | 2.8 | R196F | 2 | R73N | 1.6 | R194F | 1.4 |
| K22M | 2.7 | R196M | 1.8 | R73Y | 1.5 | R194C | 1.4 |
| K22Q | 2 | R196P | 1.7 | R73K | 1.5 | R194M | 1.4 |
| K22N | 2 | R196Q | 1.6 | R73E | 1.3 | R194K | 1.2 |
| K22P | 1.9 | R196N | 1.6 | R73P | 1.3 | R194N | 1.2 |
| K22C | 1.5 | R196K | 1.4 | R73M | 1.2 | R194E | 1.1 |
| K22D | 1.4 | R196D | 1.4 | R73C | 1.1 | R194D | 1.1 |
| K22R | 1.4 | R196E | 1.2 | R73D | 1 | R194P | 1 |
| K22E | 1.2 | R196R | 1 | R73R | 1 | R194Q | 1 |
| K22K | 1 | R196C | 0.9 | R73Q | 1 | R194R | 1 |

### Example 3: Validation of a Linker Connecting IL-35 and HSA

The inventors made different linker amino acid sequence between IL35 and HSA to compare IL-35 protein expressions.

IL-35 and HSA were connected using the linkers shown in Table 6, and the amount of IL-35-HSA secreted was evaluated. Expression vectors connecting IL-35 and HSA with each linker were constructed using the PCR method. The expression plasmids were transfected into CHO cells according to the manual of Lipofectamine 2000 (5×10⁴ cells/well, 96-well-plate, DNA 1 μg/well). The transfection method was followed the manual of Lipofectamine 2000. Transfectants were incubated for 24 hours. The culture media were centrifuged at 10,000g for 10 min and the supernatants were collected. Each secretion levels of IL-35-HSA were then analyzed by western blotting and image analysis of the obtained signal intensities was performed with ImageJ software (found online at imagej.nih.gov/ij/) to evaluate the amount of secreted proteins. The relative amount of secreted IL-35-HSA was calculated with the linker-12 as 1 (Table 6). Secretion of IL-35-HSA was significantly increased in linkers 1, 10, and 11 compared to linker 12.

TABLE 6

| | | | secretion, |
|---|---|---|---|
| | | | fold |
| Linker ID | Type | Sequence | increase |
| Linker-1 | flexible | GGGG | 3.14 |
| Linker-2 | flexible | GGGGGGGG | 1.45 |
| Linker-3 | flexible | GGGGS | 1.26 |
| Linker-4 | flexible | GGGGSGGGGS | 0.77 |
| Linker-5 | flexible | GGGGSGGGGSGGGGS | 1.19 |
| Linker-6 | rigid | PAPAP | 1.26 |
| Linker-7 | rigid | EAAAK | 1.49 |
| Linker-8 | rigid | EAAAKEAAAK | 0.89 |
| Linker-9 | rigid | EAAAKEAAAKEAAAK | 1.32 |
| Linker-10 | rigid | AEAAAKEAAAKEAAAKA | 1.73 |
| Linker-11 | rigid | AEAAAKEAAAKALEAEA | 1.64 |
| | | AAKEAAAKA | |
| Linker-12 | flexible | GGGSGGGSGGGSGGGS | 1.00 |

Table 6 shows SEQ ID NOs: 75-85 and 3, respectively.

### Example 4: Evaluation of Mutants in which Four Cysteines in EBI3 were Replaced with Serine

The inventors made cysteine substitutions to serine in EBI3 part to compare IL-35 protein expressions.

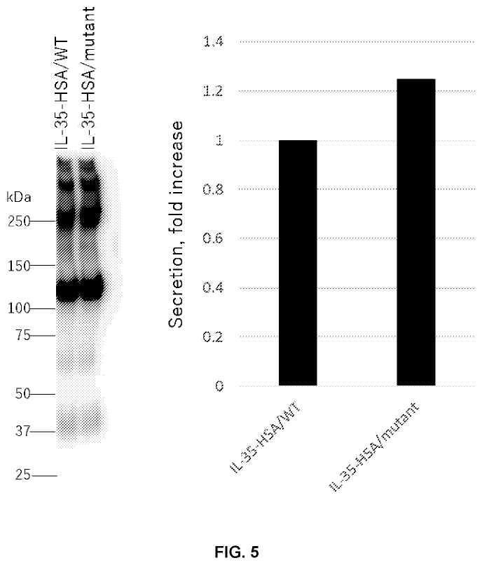

Expression vectors of the mutant, in which four cysteines in EBI3 were replaced with serine, were produced in the same manner as described in Example 1. The expression plasmids were transfected into CHO cells according to the manual of Lipofectamine 2000 (5×10⁴ cells/well, 96-well-plate, DNA 1 μg/well). The transfection method was followed the manual of Lipofectamine 2000. Transfectants were incubated for 24 hours. The culture media were centrifuged at 10,000g for 10 min and the supernatants were collected. Each secretion levels of IL-35-HSA were then analyzed by western blotting and image analysis of the obtained signal intensities was performed with ImageJ software (found online at imagej.nih.gov/ij/) to evaluate the amount of secreted proteins. The relative amount of secreted protein was calculated with the IL-35-HSA/WT as 1. Replacing cysteine with serine at four locations in EBI3 slightly increased IL-35-HSA secretion (FIG. 5).

### Example 5: Monomerization of IL-35-HSA with Additives

The inventors tested additives to prevent dimerization of IL35.

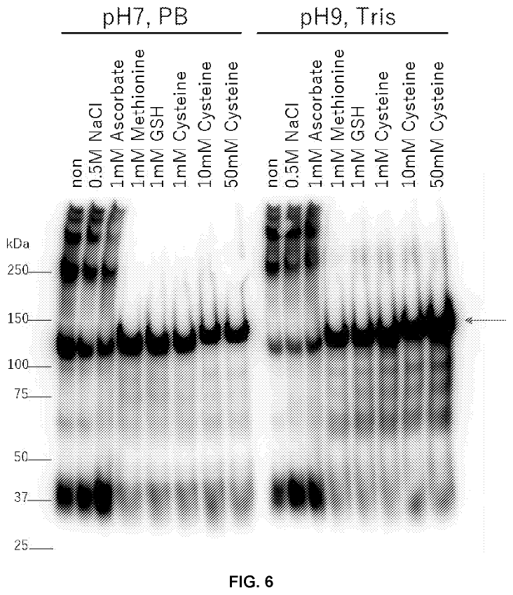

IL-35-HSA was observed to multimerize in the culture medium and during the purification process. In order to monomerize the multimerized IL-35-HSA, additives were investigated. Culture supernatants of CHO cells stably expressing IL-35-HSA were concentrated by ultrafiltration membrane and replaced with 30 mM phosphate buffer (pH 7) and 50 mM Tris buffer (pH 9), respectively. After equal amounts were dispensed into each tube, each additive was added. The additives used were ascorbic acid (final 1 mM), methionine (final 1 mM), glutathione (final 1 mM), and cysteine (final 1, 10, 50 mM), respectively. The samples were then incubated at 37° C. for 15 minutes. Each secretion profile of IL-35-HSA were then analyzed by western blotting (FIG. 6). As a result, under pH 7 and pH 9 conditions, the multimer consisting of IL-35-HSA completely disappeared upon addition of methionine, glutathione, and cysteine, and all became monomer. The inventors also tested anionic cyctlodextrins (0.001-1000 mM), reducing agents (mercaptoethanol and DTT) (0.001-1000 mM), and cationic agents (Arginine 0.001-1000 mM), PEG polymer (0.001-1000 mM).

### Example 6

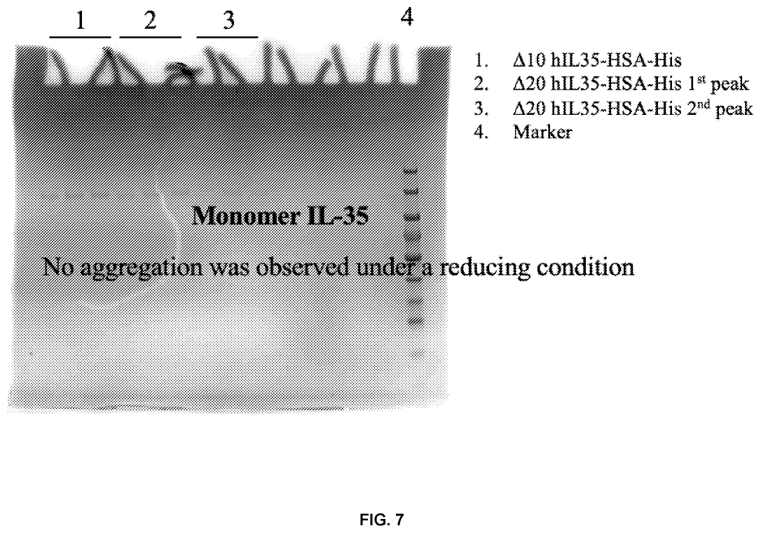

Reducing conditions can prevent aggregation and dimerization of His6-tagged IL-35-HSA. Culture supernatants of His6-tagged IL-35-HSA transfected HEK293 cells was purified using His-tag purification Ni2+column and then further purified with size exclusion column in PBS. Reducing condition of SDS-PAGE after 50 mM DTT reaction shows the multimer consisting of IL-35-HSA completely disappeared upon and all became monomer. (FIG. 7)

### Example 7 Deletion of 20 Amino Acids in the N-Terminus Ebi3

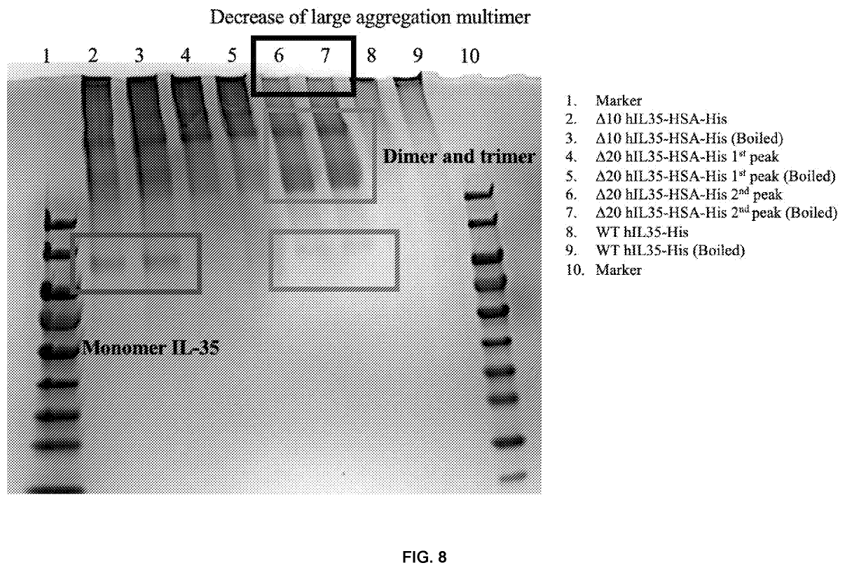

N-terminus of Ebi3 protein does not contribute to protein structure, analyzed by Alfafold2. Therefore the inventors made N-terminus deletion variants (10 amino acids deletion: delta10 and 20 amino acids deletion: delta20). Aggregation and dimerization of His6-tagged IL-35-HSA. Culture supernatants of His6-tagged IL-35-HSA transfected HEK293 cells was purified using His-tag purification Ni2+column and then further purified with size exclusion column in PBS. Non-Reducing condition of SDS-PAGE shows that 20 amino acid deletion, not 10 amino acid deletion solve the ultra-large aggregation. (FIG. 8)

### Example 8 IL-35-HSA was Detected in Lymph Node 8 Days after Subcutaneous Injection of IL-35-HSA

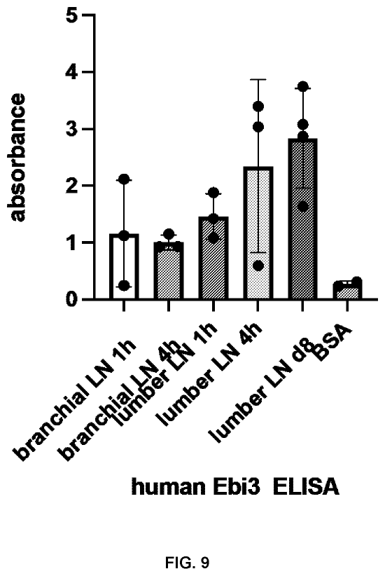

Mice were injected 40 μg of IL-35-HSA subcutaneously in the back skin. 4 hours and 8 days after injection, the inventors collected lymph nodes (FIG. 9). After hominization of lymph node, they tested the existence of human IL-35 using a ELISA kit (R and D systems DY6456-05) to detect human EBI3 protein. This kit does not detect mouse endogenous EBI3 protein. Signal of 450 nm shows that IL-35-HSA is detectable and existing in lymph nodes for long-term.

### Example 9 IL-35-HSA Injection Decreases Cytokine Release Syndrome

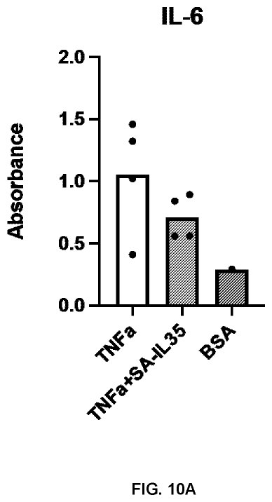

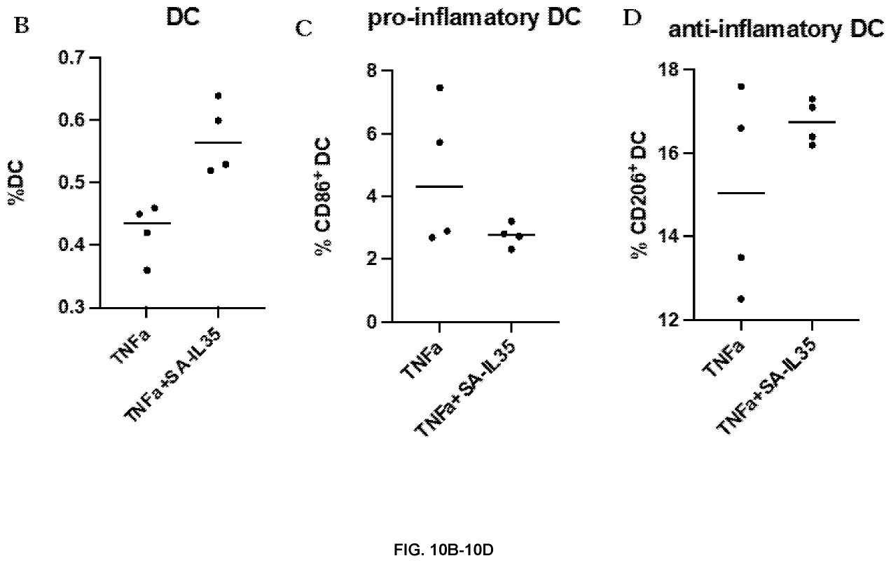

10 μg IL-35-HSA was injected twice subcutaneously on day 0 and day 7. On day 9, 2.5 μg TNF alpha protein was injected intrapreneurial. 2 hours after TNF alpha injection, blood was collected and serum IL-6 was analyzed by ELISA (FIG. 10A). TNF alpha-induced IL-6 concentration in the serum was decreased by IL-35-HSA injections. Flow cytometric analyses show that in the lymph node, dendritic cells (DCs) were increased and they show tolerogenic CD206+phenotype, rather than pro-inflammatory phenotype (FIG. 10B-D)

### Example 10 Collagen Binding Domain Fusion to IL-35 for Localized

The inventors previously found that collagen binding domain derived from von Willebrand factor A3 domain prolongs tissue retention after injection through collagen affinity in the tissue. Prolonged retention of IL-35 at the disease site may be beneficial for extending The inventors have produced collagen binding domain fused IL-35 protein (SEQ ID NOs: 53-55). Collagen-binding IL35 may be injected various injection routs including intravenously, intradermally and subcutaneously.

### Example 11 Evaluation of SA IL-35 Efficacy in the Collagen Antibody Induced Arthritis Model

### A. Results:

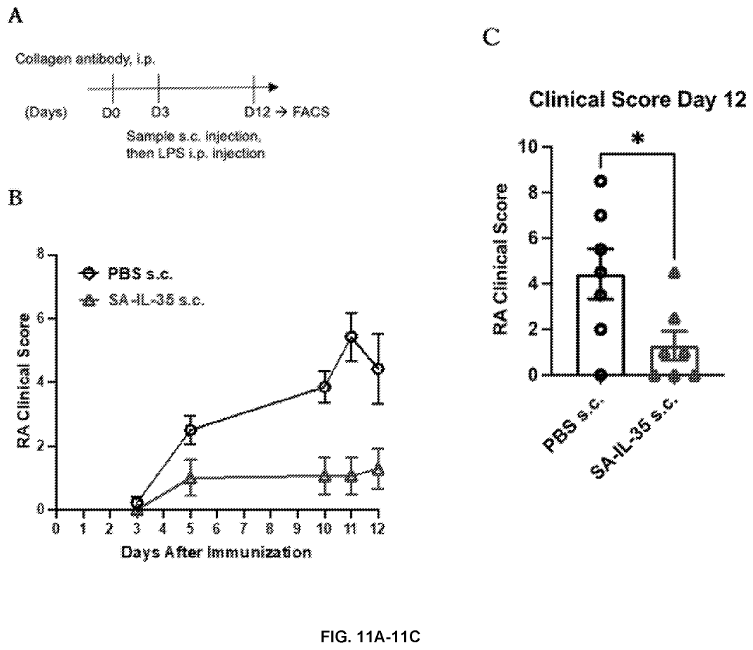

CHO-cell derived mutated SA IL-35 supernatant was purified via cation exchange in 50 mM acetic acid buffer (pH 5) and eluted with 50 mM acetic acid with 1M NaCl (pH 5). The resultant protein was further purified via size exclusion chromatography and is depicted in FIG. 1. Mutated SA IL-35 was then evaluated as a prophylactic treatment to prevent the onset of collagen antibody induced arthritis (CAIA). The CAIA model was induced, as depicted in FIG. 11A, on day 0, by intraperitoneally injecting a collagen-II antibody cocktail in 8-week-old female BALB/c mice (Jackson laboratories). The antibody cocktail consists of four arthritis-inducing monoclonal antibodies that are specific for epitopes located on type II collagen [1]. Antibody binding to the epitopes induces immune complex formation, followed by deposition in the cartilage and the synovium and results in a complement-dependent, inflammatory reaction that causes joint swelling [1]. On day 3 after collagen II antibody immunization, CAIA mice were treated with 40 μg mutated SA IL-35 (wild type IL-35 molar equivalent) by subcutaneous injection in the flank of the back, followed by an intraperitoneal injection of 25 μg LPS. From day 3 until day 12 the clinical score of the mice's front and hind paws were recorded daily. The severity of joint inflammation ranged from a score 0 to 4 where a score of 0 refers to healthy paw, 1 refers to swelling and/or redness in one joint, 2 refers to swelling and/or redness in more than one joint, 3 refers to swelling and/or redness in the entire paw, and 4 refers to maximal swelling. The clinical score results of the experiment are depicted in FIGS. 11B-C. Prophylactic treatment with a single dose of mutated SA IL-35 prevented the onset of severe disease in the CAIA mouse model of arthritis.

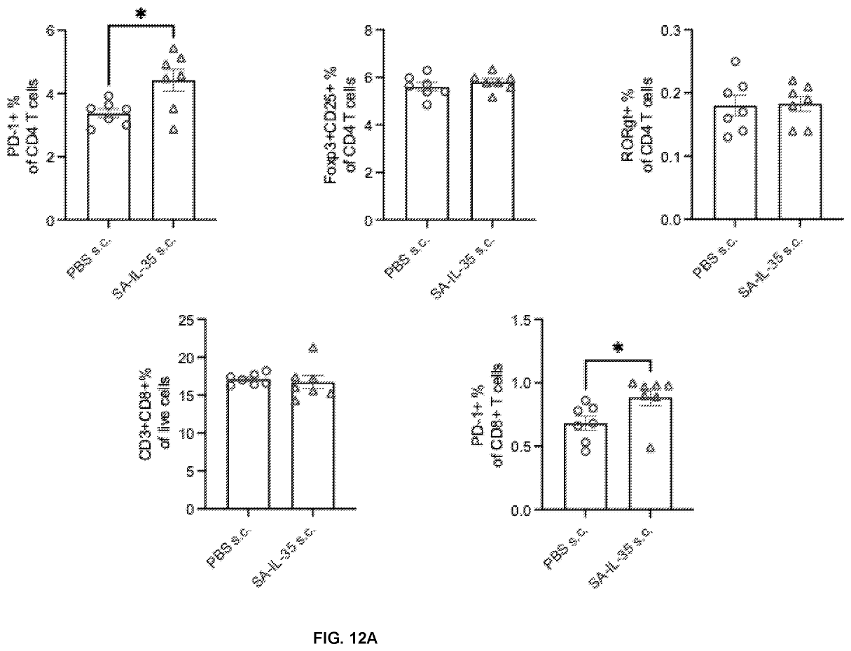

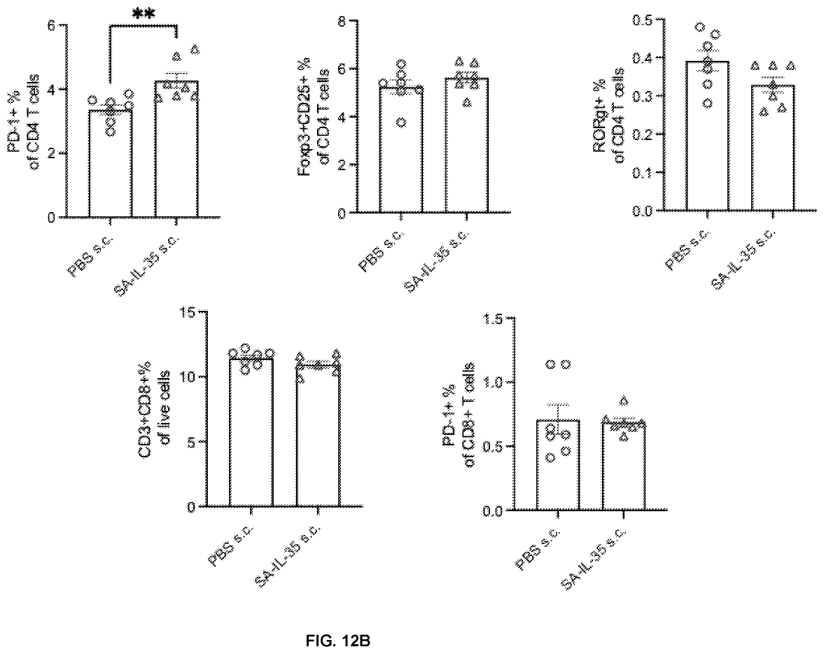

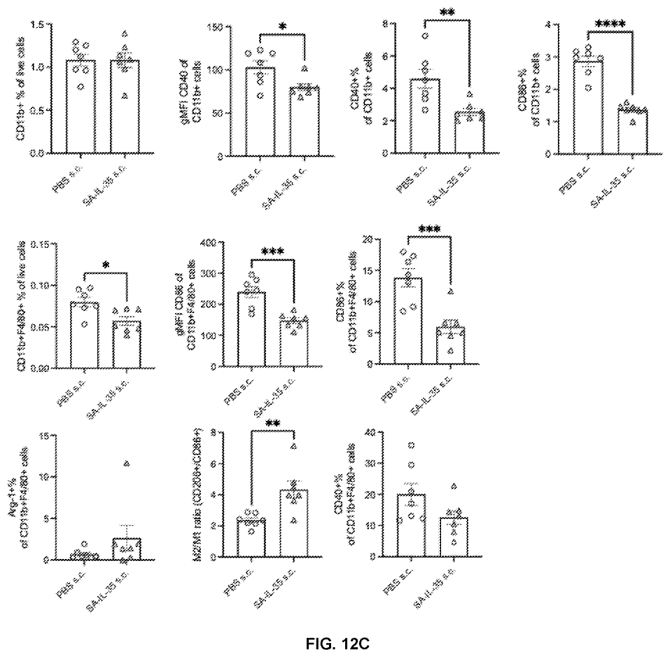

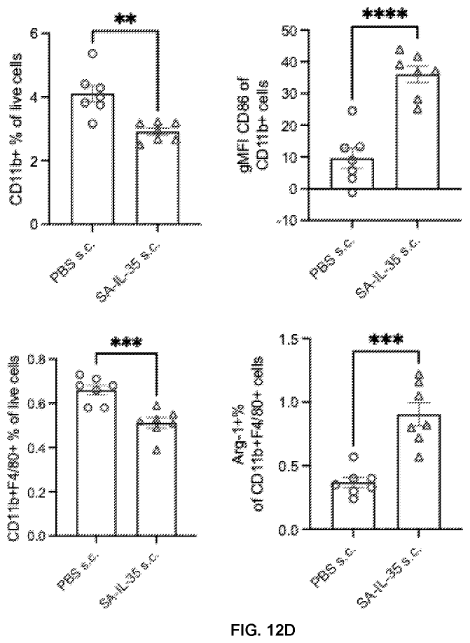

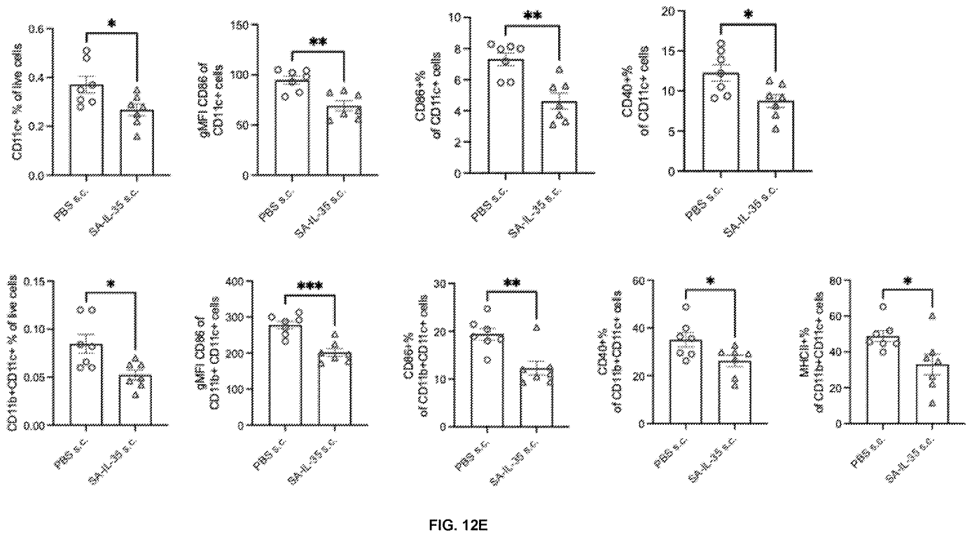

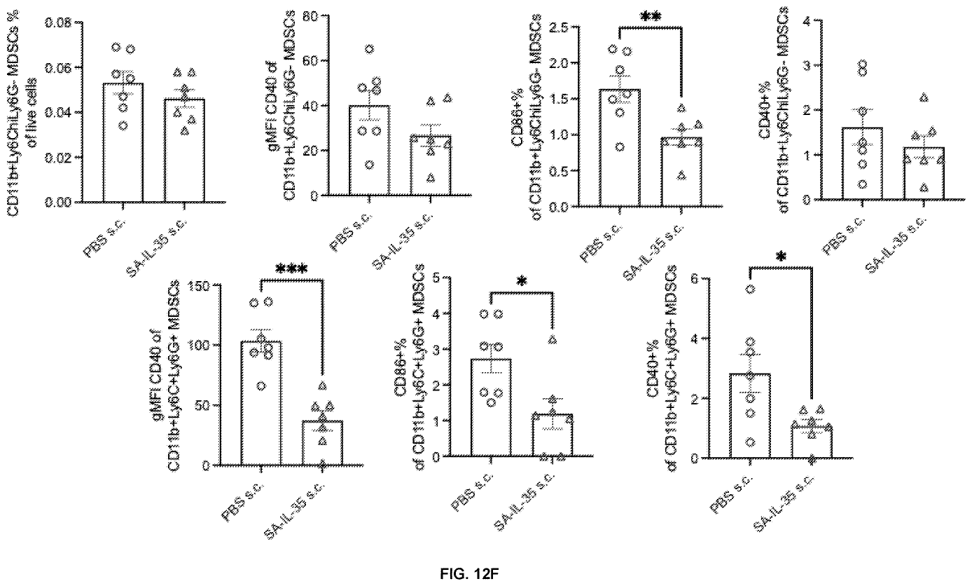

To evaluate the effect of mutated SA IL-35 on immune cell populations in the secondary lymphoid organs, single-cell suspensions from the draining lymph nodes (popliteal) and spleen were harvested at endpoint, and stained for various markers of T cell and myeloid phenotype and activation as described previously [2]. In the T-cell compartment, mutated SA IL-35 treatment elevated the expression of PD-1 in CD4+ and CD8+ T cells in the lymph nodes (FIG. 12A), as well as the expression of PD-1 in CD4+ T cells in the spleen (FIG. 12B). In the myeloid compartment, mutated SA IL-35 treatment reduces the expression of CD40 and CD86 on CD11b+ myeloid cells in the lymph nodes (FIG. 12C). Treatment also reduces the percentage of CD11b+F4/80+macrophages in the lymph nodes as well as the expression of CD86 on macrophages, while increasing the M2 to M1 macrophage ratio (FIG. 12C). In the spleen, mutated SA IL-35 treatment reduces the percentage of CD11b+myeloid cells and the expression of CD86 on CD11b+ cells (FIG. 12D). Treatment also reduces the percentage of CD11b+F4/80+ macrophages while increasing the percentage of Arg1+M2 macrophages (FIG. 12D). In the dendritic cell compartment, mutated SA IL-35 treatment reduces the percentage of CD11c+ dendritic cells and the expression of CD86 and CD40 on CD11c+ dendritic cells in the lymph nodes (FIG. 12E). Treatment also reduces the percentage of CD11c+CD11b+ dendritic cells and the expression of CD86, CD40 and MHC class II on these cells (FIG. 12E). In the myeloid-derived suppressor cell compartment, mutated SA IL-35 treatment reduced the expression of CD86 and CD40 on CD11b+Ly6Chi Ly6G-MDSCs (FIG. 12F).

### B. Methods:

1. Collagen-Induced Arthritis Model: (Adapted from Yuba et al 2021)

Arthritis was induced in 8-week-old female BALB/c mice (Jackson Labs) as descripted previously [2]. On day 0, mice were intraperitoneally injected with 1 mg/mouse anti-collagen antibody cocktail (Chondrex), followed by 25 μg lipopolysaccharide (Chondrex). On day 3 after collagen II antibody immunization, CAIA mice were treated with 40 μg mutated SA IL-35 mutated SA IL-35 (wild type IL-35 molar equivalent) by subcutaneous injection in the flank of the back, followed by an intraperitoneal injection of 25 μg LPS. From day 3 until day 12 the clinical score of the mice's front and hind paws were recorded daily according to the manufacturer's protocol (Chondrex).

### 2. Flow Cytometric Analysis:

The popliteal lymph node and spleen tissues were digested in DMEM medium supplemented with 2% FBS, 2 mg ml-1 collagenase D (Sigma-Aldrich) for 45 min at 37° C. Single-cell suspensions were obtained by gentle disruption through a 70-μm cell strainer. For the spleen, red blood cells in the blood were lysed with ACK lysing buffer (Quality Biological), followed by antibody staining for flow cytometry. Cells were blocked with CD16/32 antibody and stained with Live/dead fixable aqua (eBioscience) on ice for 20 minutes according to the manufacturer's instructions. Following a washing step, the cells were then stained with surface antibodies for 20 min on ice. Intracellular staining was performed using a FoxP3 staining kit according to the manufacturer's instructions (BioLegend). After washing once, the cells were stained with intracellular antibodies overnight at room temperature. Flow cytometric analysis was performed using a Fortessa flow cytometer (BD Biosciences) and analyzed using FlowJo software (Tree Star).

Although certain embodiments have been described above with a certain degree of particularity, or with reference to one or more individual embodiments, those skilled in the art could make numerous alterations to the disclosed embodiments without departing from the scope of this invention. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments. Any reference to a patent publication or other publication is a herein a specific incorporation by reference of the disclosure of that publication. The claims are not to be interpreted as including means-plus- or step-plus-function limitations, unless such a limitation is explicitly recited in a given claim using the phrase(s) “means for” or “step for,” respectively.

### REFERENCES

[1] Chondrex, Inc. (2023). Collagen antibody induced arthritis. Chondrex. Retrieved from https://www.chondrex.com/collagen-antibody-arthritis
[2] Yuba, E., Budina, E., Katsumata, K., Ishihara, A., Mansurov, A., Alpar, A. T., Watkins, E. A., Hosseinchi, P., Reda, J. W., Lauterbach, A. L., Nguyen, M., Solanki, A., Kageyama, T., Swartz, M. A., Ishihara, J., & Hubbell, J. A. (2021). Suppression of Rheumatoid Arthritis by Enhanced Lymph Node Trafficking of Engineered Interleukin-10 in Murine Models. Arthritis and Rheumatology, 73 (5), pp. 769-778.

1. A polypeptide comprising the amino acid sequence of SEQ ID NO:2 with one or more amino acid substitutions relative to SEQ ID NO:2, wherein the one or more amino acid substitutions comprise K23, R74, R195, R197, or combinations thereof.

2. The polypeptide of claim 1, wherein the polypeptide comprises an amino acid sequence that has at least 70% sequence identity to SEQ ID NO:2.

3. The polypeptide of claim 1 or 2, wherein the polypeptide comprises substitutions of one or more of C36, C47, C80, and C90, relative to SEQ ID NO:2.

4. The polypeptide of claim 3, wherein the substitution(s) are with serine.

5. A polypeptide comprising the amino acid sequence of SEQ ID NO:4 with one or more amino acid substitutions relative to SEQ ID NO:4, wherein the one or more amino acid substitutions comprise C74.

6. The polypeptide of claim 5, wherein the polypeptide comprises an amino acid sequence that has at least 70% sequence identity to SEQ ID NO:4.

7. A polypeptide comprising the amino acid sequence of SEQ ID NO:6 with one or more amino acid substitutions relative to SEQ ID NO:6, wherein the one or more amino acid substitutions comprise C34.

8. The polypeptide of claim 7, wherein the polypeptide comprises an amino acid sequence that has at least 70% sequence identity to SEQ ID NO:6.

9. A polypeptide comprising the amino acid sequence of SEQ ID NO:90 with one or more amino acid substitutions relative to SEQ ID NO:90, wherein the one or more amino acid substitutions comprise K2, R53, R174, R176, or combinations thereof.

10. The polypeptide of claim 9, wherein the polypeptide comprises an amino acid sequence that has at least 70% sequence identity to SEQ ID NO:90.

11. The polypeptide of claim 9 or 10, wherein the polypeptide comprises substitutions of one or more of C15, C26, C59, and C69, relative to SEQ ID NO:90.

12. The polypeptide of claim 11, wherein the substitution(s) are with serine.

13. A polypeptide comprising an EBI3 subunit of IL-35, wherein the EBI3 subunit comprises fewer than 200 amino acids and comprises the amino acid sequence of SEQ ID NO: 92 or an amino acid sequence having at least 70% sequence identity to SEQ ID NO:92.

14. The polypeptide of claim 13, wherein the EBI3 subunit comprises one or more amino acid substitutions and wherein the one or more amino acid substitutions comprise R33, R154, R156, or combinations thereof, relative to SEQ ID NO:92.

15. The polypeptide of claim 13 or 14, wherein the EBI3 subunit consists of 189 amino acids.

16. The polypeptide of any one of claims 13-15, wherein the polypeptide comprises substitutions of C6, C39, C49, or combinations thereof, relative to SEQ ID NO:92.

17. The polypeptide of claim 16, wherein the substitution(s) are with serine.

18. A polypeptide comprising the amino acid sequence of SEQ ID NO:1 with one or more amino acid substitutions relative to SEQ ID NO:1, wherein the one or more amino acid substitutions comprise K23, R74, R195, R197, C320, C497, or combinations thereof.

19. The polypeptide of claim 18, wherein the polypeptide comprises an amino acid sequence that has at least 70% sequence identity to SEQ ID NO:1.

20. The polypeptide of claim 18 or 19, wherein the one or more amino acid substitutions comprise K23, C320, and C497, relative to SEQ ID NO:1.

21. A polypeptide comprising the amino acid sequence of SEQ ID NO:91 with one or more amino acid substitutions relative to SEQ ID NO:91, wherein the one or more amino acid substitutions comprise K2, R53, R174, R176, C299, C476, or combinations thereof.

22. The polypeptide of claim 21, wherein the polypeptide comprises an amino acid sequence that has at least 70% sequence identity to SEQ ID NO:91.

23. The polypeptide of claim 21 or 22, wherein the one or more amino acid substitutions comprise K2, C299, and C476, relative to SEQ ID NO:91.

24. The polypeptide of any one of claims 21-23, wherein the polypeptide further comprises amino acid substitutions of C15, C26, C59, C69, or combinations thereof, relative to SEQ ID NO: 91.

25. The polypeptide of claim 24, wherein the amino acid substitution is with a serine.

26. The polypeptide of any one of claims 1-25, wherein the polypeptide further comprises an ECM-affinity peptide, serum protein, albumin binding protein (ABP), and/or Fc peptide.

27. The polypeptide of claim 26, wherein the ECM-affinity peptide comprises an amino acid sequence of one of SEQ ID NOs: 9-27 or 88 or an amino acid sequence having at least 70% sequence identity to one of SEQ ID NOs: 9-27 or 88.

28. The polypeptide of claim 26 or 27, wherein the Fc peptide comprises an amino acid sequence of SEQ ID NO:86 or 87 or an amino acid sequence having at least 70% sequence identity to SEQ ID NO:86 or 87.

29. The polypeptide of any one of claims 26-28, wherein the ABP comprises the amino acid sequence of SEQ ID NO:93 or an amino acid sequence having at least 70% sequence identity to SEQ ID NO:93.

30. The polypeptide of any one of claims 26-29, wherein the ECM-affinity peptide, serum protein, ABP, and/or Fc peptide is amino-proximal to the polypeptide comprising the substituted amino acid sequence of one of SEQ ID NO:1, 2, 4, 6, 90, 91 or the EBI3 subunit.

31. The polypeptide of any one of claims 26-28, wherein the ECM-affinity peptide, serum protein, and/or Fc peptide is carboxy-proximal to the polypeptide comprising the substituted amino acid sequence of one of SEQ ID NO:1, 2, 4, 6, 90, 91 or the EBI3 subunit.

32. The polypeptide of any one of claims 26-31, wherein the serum protein comprises a polypeptide having the amino acid sequence of SEQ ID NO:6 or a polypeptide with at least 70% sequence identity to a polypeptide having the amino acid sequence of SEQ ID NO:6.

33. The polypeptide of claim 32, wherein the polypeptide comprises one or more amino acid substitutions relative to SEQ ID NO:6, wherein the one or more amino acid substitutions comprise C34.

34. The polypeptide of claim 33, wherein the amino acid substitution at C34 is with an alanine or serine amino acid residue.

35. A chimeric or multimeric polypeptide comprising:
(i) the amino acid sequence of SEQ ID NO:2 with one or more amino acid substitutions relative to SEQ ID NO:2, wherein the one or more amino acid substitutions relative to SEQ ID NO: 2 comprise K23, R74, R195, R197, or combinations thereof; and
(ii) the polypeptide of SEQ ID NO:4, a polypeptide having at least 70% sequence identity to SEQ ID NO:4; or a polypeptide comprising SEQ ID NO:4 with one or more substitutes relative to SEQ ID NO:4.

36. The chimeric or multimeric polypeptide of claim 35, wherein the polypeptide comprising a substituted amino acid sequence of SEQ ID NO:2 comprises at least 70% sequence identity to SEQ ID NO:2.

37. The chimeric or multimeric polypeptide of claim 35 or 36, wherein the polypeptide comprises substitutions of C36, C47, C80, C90, or combinations thereof, relative to the amino acid sequence of SEQ ID NO:2.

38. The polypeptide of claim 37, wherein the amino acid substitution(s) are with serine.

39. The chimeric or multimeric polypeptide of any one of claims 35-38, wherein the polypeptide comprises a polypeptide comprising SEQ ID NO:4 with one or more amino acid substitutions relative to SEQ ID NO:4 and wherein the one or more amino acid substitutions comprise C74.

40. The chimeric or multimeric polypeptide of any one of claims 35-39, wherein the polypeptide comprising the substituted amino acid sequence of SEQ ID NO:2 is amino-proximal to the polypeptide with the amino acid sequence of SEQ ID NO:4, the polypeptide with an amino acid sequence having at least 70% sequence identity to SEQ ID NO:4, or the polypeptide comprising the substituted amino acid sequence of SEQ ID NO:4.

41. The chimeric or multimeric polypeptide of any one of claims 35-39, wherein the polypeptide comprising the substituted amino acid sequence of SEQ ID NO:2 is carboxy-proximal to the polypeptide with the amino acid sequence of SEQ ID NO:4, the polypeptide with an amino acid sequence having at least 70% sequence identity to SEQ ID NO:4, or the polypeptide comprising the substituted amino acid sequence of SEQ ID NO:4.

42. The chimeric or multimeric polypeptide of any one of claims 35-41, wherein the one or more amino acid substitutions relative to SEQ ID NO:2 comprise K23 and wherein the one or more amino acid substitutions relative to SEQ ID NO:4 comprise C74.

43. The chimeric or multimeric polypeptide of claim 42, wherein the amino acid substitutions at K23 and/or C74 are substitutions with an alanine and/or serine amino acid residue.

44. The chimeric or multimeric polypeptide of claim 43, wherein the substitutions comprise K23A and C74S.

45. A chimeric or multimeric polypeptide comprising:
(i) the amino acid sequence of SEQ ID NO:90 with one or more amino acid substitutions relative to SEQ ID NO:90, wherein the one or more amino acid substitutions relative to SEQ ID NO:90 comprise K2, R53, R174, R176, or combinations thereof; and
(ii) the polypeptide of SEQ ID NO:4, a polypeptide having at least 70% sequence identity to SEQ ID NO:4; or a polypeptide comprising SEQ ID NO:4 with one or more amino acid substitutions relative to SEQ ID NO:4.

46. The chimeric or multimeric polypeptide of claim 45, wherein the polypeptide comprising a substituted amino acid sequence of SEQ ID NO:90 comprises at least 70% sequence identity to SEQ ID NO:90.

47. The chimeric or multimeric polypeptide of claim 45 or 46, wherein the polypeptide comprises amino acid substitutions of C15, C26, C59, C69, or combinations thereof, relative to SEQ ID NO:90.

48. The chimeric or multimeric polypeptide of claim 47, wherein the amino acid substitution(s) are with serine.

49. The chimeric or multimeric polypeptide of any one of claims 45-48, wherein the polypeptide comprises a polypeptide comprising SEQ ID NO:4 with one or more substitutes relative to SEQ ID NO:4 and wherein the one or more amino acid substitutions comprise C74.

50. The chimeric or multimeric polypeptide of any one of claims 45-49, wherein the polypeptide comprising the substituted amino acid sequence of SEQ ID NO:90 is amino-proximal to the polypeptide with the amino acid sequence of SEQ ID NO:4, the polypeptide with an amino acid sequence having at least 70% sequence identity to SEQ ID NO:4, or the polypeptide comprising the substituted amino acid sequence of SEQ ID NO:4.

51. The chimeric or multimeric polypeptide of any one of claims 45-49, wherein the polypeptide comprising the substituted amino acid sequence of SEQ ID NO:90 is carboxy-proximal to the polypeptide with the amino acid sequence of SEQ ID NO:4, the polypeptide with an amino acid sequence having at least 70% sequence identity to SEQ ID NO:4, or the polypeptide comprising the substituted amino acid sequence of SEQ ID NO:4.

52. The chimeric or multimeric polypeptide of any one of claims 45-51, wherein the one or more amino acid substitutions relative to SEQ ID NO:90 comprises K2 and wherein the one or more amino acid substitutions relative to SEQ ID NO:4 comprises C74.

53. The chimeric or multimeric polypeptide of claim 52, wherein the substitutions at K2 and/or C74 are amino acid substitutions with an alanine and/or serine amino acid residue.

54. The chimeric or multimeric polypeptide of claim 53, wherein the substitutions comprise K2A and C74S.

55. A chimeric or multimeric polypeptide comprising:
(i) an EBI3 subunit of IL-35, wherein the EBI3 subunit comprises less than 200 amino acids and comprises the amino acid sequence of SEQ ID NO:92 or an amino acid sequence having at least 70% sequence identity ot SEQ ID NO:92 and
(ii) the polypeptide of SEQ ID NO:4, a polypeptide having at least 70% sequence identity to SEQ ID NO:4; or a polypeptide comprising SEQ ID NO:4 with one or more amino acid substitutions relative to SEQ ID NO:4.

56. The chimeric or multimeric polypeptide of claim 55, wherein the EBI3 subunit comprises one or more amino acid substitutions and wherein the one or more amino acid substitutions comprise R33, R154, R156, or combinations thereof, relative to SEQ ID NO:92.

57. The chimeric or multimeric polypeptide of claim 55 or 56, wherein the EBI3 subunit consists of 189 amino acids.

58. The chimeric or multimeric polypeptide of any one of claims 55-57, wherein the EBI3 subunit comprises substitutions of C6, C39, C49, or combinations thereof, relative to SEQ ID NO: 92.

59. The chimeric or multimeric polypeptide of claim 58, wherein the amino acid substitution(s) are with a serine.

60. The chimeric or multimeric polypeptide of any one of claims 55-59, wherein the polypeptide comprises a polypeptide comprising SEQ ID NO:4 with one or more amino acid substitutions relative to SEQ ID NO:4 and wherein the one or more amino acid substitutions comprise C74.

61. The chimeric or multimeric polypeptide of claim 60, wherein the amino acid substitution comprises C74S.

62. The chimeric or multimeric polypeptide of any one of claims 55-61, wherein the EBI3 subunit is amino-proximal to the polypeptide with the amino acid sequence of SEQ ID NO:4, the polypeptide with an amino acid sequence having at least 70% sequence identity to SEQ ID NO: 4, or the polypeptide comprising the substituted amino acid sequence of SEQ ID NO:4.

63. The chimeric or multimeric polypeptide of any one of claims 55-60, wherein the EBI3 subunit is carboxy-proximal to the polypeptide with the amino acid sequence of SEQ ID NO:4, the polypeptide with an amino acid sequence having at least 70% sequence identity to SEQ ID NO: 4, or the polypeptide comprising the substituted amino acid sequence of SEQ ID NO:4.

64. The chimeric or multimeric polypeptide of any one of claims 35-63, wherein the chimeric polypeptide comprises a linker between (i) and (ii).

65. The chimeric or multimeric polypeptide of claim 64, wherein the linker comprises or consists of the amino acid sequence of one of SEQ ID NOs: 3, 5, 7, 8, or 75-85.

66. The chimeric or multimeric polypeptide of claim 64 or 65, wherein the linker comprises glycine and serine residues.

67. The chimeric or multimeric polypeptide of claim 66, wherein the linker comprises a (G3S) n linker, wherein n is an integer from 1 to 10.

68. The chimeric or multimeric polypeptide of claim 67, wherein the linker comprises or consists of the amino acid sequence of SEQ ID NO:3.

69. The chimeric or multimeric polypeptide of any one of claims 35-68, wherein the chimeric polypeptide further comprises a serum albumin (SA) polypeptide.

70. The chimeric or multimeric polypeptide of claim 69, wherein the SA polypeptide comprises a human SA (HSA) polypeptide.

71. The chimeric or multimeric polypeptide of claim 69 or 70, wherein the SA polypeptide comprises a polypeptide having the amino acid sequence of SEQ ID NO:6 or a polypeptide with at least 70% sequence identity to a polypeptide with the amino acid sequence of SEQ ID NO: 6.

72. The chimeric or multimeric polypeptide of claim 71, wherein the polypeptide comprises one or more amino acid substitutions relative to the amino acid sequence of SEQ ID NO:6, wherein the one or more amino acid substitutions comprise C34.

73. The chimeric or multimeric polypeptide of claim 72, wherein the amino acid substitution at C34 is with an alanine or serine amino acid residue.

74. The chimeric or multimeric polypeptide of any one of claims 69-72, wherein the SA polypeptide is carboxy-proximal to (i) and/or (ii).

75. The chimeric or multimeric polypeptide of any one of claims 69-72, wherein the SA polypeptide is amino-proximal to (i) and/or (ii).

76. The chimeric or multimeric polypeptide of any one of claims 69-75, wherein the chimeric polypeptide comprises a linker between the SA polypeptide and the polypeptide of (i) and/or (ii).

77. The chimeric or multimeric polypeptide of claim 76, wherein the linker comprises or consists of one of SEQ ID NOs: 3, 5, 7, 8, or 75-85.

78. The chimeric or multimeric polypeptide of claim 76 or 77, wherein the linker comprises glycine and serine residues.

79. The chimeric or multimeric polypeptide of claim 78, wherein the linker comprises a (G3S) n linker, wherein n is an integer from 1 to 10 or a linker of SEQ ID NO:75.

80. The chimeric or multimeric polypeptide of any one of claims 76-79, wherein the linker comprises SEQ ID NO:5 or SEQ ID NO:75.

81. The chimeric or multimeric polypeptide of any one of claims 76-80, wherein the linker consists of SEQ ID NO:5 or SEQ ID NO:75.

82. The chimeric or multimeric polypeptide of any one of claims 35-81, wherein the chimeric polypeptide comprises an amino acid sequence having at least 70% sequence identity to SEQ ID NO:1 or 91.

83. The chimeric or multimeric polypeptide of any one of claims 35-82, wherein the chimeric or multimeric polypeptide further comprises an ECM-affinity peptide.

84. The chimeric or multimeric polypeptide of claim 83, wherein the ECM-affinity peptide is carboxy-proximal to (i) and/or (ii).

85. The chimeric or multimeric polypeptide of claim 83, wherein the ECM-affinity peptide is amino-proximal to (i) and/or (ii).

86. The chimeric or multimeric polypeptide of any one of claims 83-85, wherein the ECM-affinity peptide comprises the amino acid sequence of one of SEQ ID NOs: 9-27 or 88 or an amino acid sequence that has at least 70% sequence identity to one of SEQ ID NOs: 9-27 or 88.

87. The chimeric or multimeric polypeptide of any one of claims 83-86, wherein the chimeric polypeptide comprises a linker between the ECM-affinity peptide polypeptide and (i) and/or (ii).

88. The chimeric or multimeric polypeptide of claim 87, wherein the linker comprises or consists of one of SEQ ID NOs: 3, 7, 8, or 75-85.

89. The chimeric or multimeric polypeptide of any one of claims 35-88, wherein the chimeric or multimeric polypeptide further comprises a Fc peptide.

90. The chimeric or multimeric polypeptide of claim 89, wherein the Fc peptide is carboxy-proximal to to (i) and/or (ii).

91. The chimeric or multimeric polypeptide of claim 89, wherein the Fc peptide is amino-proximal to (i) and/or (ii).

92. The chimeric or multimeric polypeptide of any one of claims 89-91, wherein the Fc peptide comprises the amino acid sequence of SEQ ID NO:86 or 87 or an amino acid sequence that has at least 70% sequence identity to SEQ ID NO:86 or 87.

93. The chimeric or multimeric polypeptide of any one of claims 89-92, wherein the chimeric polypeptide comprises a linker between the Fc peptide and (i) and/or (ii).

94. The chimeric or multimeric polypeptide of claim 93, wherein the linker comprises or consists of one of SEQ ID NOs: 3, 7, 8, or 75-85.

95. The chimeric or multimeric polypeptide of any one of claims 35-94, wherein the chimeric or multimeric polypeptide further comprises an albumin binding protein (ABP).

96. The chimeric or multimeric polypeptide of claim 89, wherein the ABP is carboxy-proximal to (i) and/or (ii).

97. The chimeric or multimeric polypeptide of claim 89, wherein the ABP is amino-proximal to (i) and/or (ii).

98. The chimeric or multimeric polypeptide of any one of claims 95-97, wherein the ABP comprises the amino acid sequence of SEQ ID NO:93 or an amino acid sequence having at least 70% sequence identity to SEQ ID NO:93.

99. The chimeric or multimeric polypeptide of any one of claims 95-98, wherein the chimeric polypeptide comprises a linker between the ABP and (i) and/or (ii).

100. The chimeric or multimeric polypeptide of claim 99, wherein the linker comprises or consists of one of SEQ ID NOs: 3, 7, 8, or 75-85.

101. The polypeptide of any one of claims 1-100, wherein the polypeptide comprises the amino acid sequence of one of SEQ ID NOs: 1-93 or an amino acid sequence having at least 70% sequence identity to one of SEQ ID NOs: 1-93.

102. The polypeptide of any one of claims 1-101, wherein the substitution is a substitution with a nonpolar amino acid.

103. The polypeptide of any one of claims 1-102, the substitution is a substitution with alanine, leucine, isoleucine, proline, tryptophan, valine, phenylalanine, or methionine.

104. The polypeptide of claim 103, wherein the substitution is a amino acid substitution with an alanine.

105. The polypeptide of any one of claims 1-102, wherein the amino acid substitution is a substitution with a serine or glycine.

106. The polypeptide of any one of claims 1-102, wherein the amino acid substitution excludes a substitution with a lysine or arginine.

107. A nucleic acid encoding the polypeptide of any one of claims 1-106.

108. An expression vector comprising the nucleic acid of claim 107.

109. A host cell comprising the polypeptide of any one of claims 1-103, the nucleic acid of claim 107, or the expression vector of claim 108.

110. A pharmaceutical composition comprising the polypeptide of any one of claims 1-106, the nucleic acid of claim 107, the expression vector of claim 108, or the host cell of claim 109.

111. A method of making a cell comprising transferring the nucleic acid of claim 107 or the expression vector of claim 108 into a cell.

112. A method for making a polypeptide comprising transferring the expression vector of claim 108 into a cell and incubating the cell under conditions sufficient for expression of the polypeptide encoded on the expression vector.

113. A method for treating an autoimmune or inflammatory condition in a subject comprising administering the polypeptide of any one of claims 1-106 or the pharmaceutical composition of claim 110 to the subject.

114. A method for reducing inflammation, for delivering cytokine therapy to lymph nodes, and/or for increasing tolerogenic dendritic cells in the lymph nodes in a subject comprising administering the polypeptide of any one of claims 1-106 or the pharmaceutical composition of claim 110 to the subject.

115. The method of claim 114, wherein the inflammation is associated with an autoimmune or inflammatory condition.

116. The method of claim 113 or 115, wherein the autoimmune or inflammatory condition comprises cytokine release syndrome, cytokine storm, arthritis, inflammatory bowel disease, scleroderma, inflammatory bowel disease, idiopathic pulmonary fibrosis, multiple sclerosis, type 1 diabetes, Crohn's disease, psoriasis, acute inflammation, chronic inflammation, neuroinflammation, acute respiratory distress syndrome, rheumatoid arthritis, fibrosis, infection, allergy, inflammatory therapy-related adverse events, ischemic stroke, pulmonary hypertension, ischemia, cardiac stroke, brain stroke, atherosclerosis, wound healing, peripheral artery disease, cerebral ischemia-reperfusion (I/R) injury, myocardial infarction, and related inflammatory illness.

117. The method of claim 116, wherein the autoimmune or inflammatory condition comprises multiple sclerosis.

118. The method of claim 116, wherein the autoimmune or inflammatory condition comprises rheumatoid arthritis.

119. The method of claim 116, wherein the autoimmune or inflammatory condition comprises cytokine release syndrome.

120. The method of any one of claims 113-118, wherein the composition is administered systemically.

121. The method of claim 120, wherein the composition is administered by intravenous injection.

122. The method of any one of claims 113-118, wherein the composition is administered locally.

123. The method of claim 122, wherein the composition is administered to or adjacent to a site of inflammation.

124. The method of any one of claims 113-123, wherein the subject has been previously treated with an anti-inflammatory agent, anti-inflammatory therapy, or autoimmune therapy.

125. The method of claim 124, wherein the subject has been determined to be non-responsive to the previous treatment.

126. The method of any one of claims 113-123, wherein the subject has not been treated previously for the inflammatory or autoimmune disease.

127. The method of any one of claims 113-126, wherein the method further comprises administration of an additional inflammatory or autoimmune therapy.

128. The method of claim 127, wherein the additional inflammatory or autoimmune therapy comprises an anti-cytokine agent or a steroid.

129. The method of claim 128, wherein the additional inflammatory or autoimmune therapy comprises one or more of steroids, corticosteroids, anti-TNF-alpha therapy, anti-integrin therapy, infliximab, mesalamine, and vedolizumab, fingolimod, interferon-β, dimethyl fumarate, teriflunomide, integrin α4β1, an anti-αLβ2 antibody, an anti-IL-6R agent, an anti-IL-6 agent, and a Janus kinase inhibitor (e.g., tofacitinib, baricitinib, upadacitinib).

130. The method of claim 129, wherein the steroid comprises dexamethasone, methylprednisolone, and/or prednisolone

131. The method of any one of claims 113-130, wherein the subject is a human subject.

132. The method of any of claims 113-131, wherein the polypeptide or composition decreases a number of Th17 cells in the subject.

133. The method of any of claims 113-132, wherein the polypeptide or composition inhibits a function of Th17 cells in the subject.

134. The method of any of claims 113-133, wherein the polypeptide or composition is administered to the subject via a pre-filled syringe.

135. The method of any of claims 113-134, wherein the polypeptide or composition is administered at a dose of between 0.1 mg/kg and 50 mg/kg.

136. The method of any of claims 113-135, further comprising detecting the anti-inflammatory cytokine in a lymph node of the subject.

137. The method of claim 136, wherein the detecting comprises obtaining a lymph sample from the subject.

138. The method of claim 137, wherein the detecting comprises detecting the presence of the anti-inflammatory cytokine in the lymph sample.


SEQUENCE LISTING (93 sequences)

SEQ ID NO: 1 - protein, 1048 aa, synthetic construct
METDTLLLWVLLLWVPGSTGDRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSGGGSGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 2 - protein, 230 aa, synthetic construct
METDTLLLWVLLLWVPGSTGDRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 3 - protein, 16 aa, synthetic construct
GGGSGGGSGGGSGGGS

SEQ ID NO: 4 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 5 - protein, 20 aa, synthetic construct
GGGSGGGSGGGSGGGSGGGS

SEQ ID NO: 6 - protein, 585 aa, synthetic construct [sequence omitted]

SEQ ID NO: 7 - protein, 8 aa, synthetic construct
GGGSGGGS

SEQ ID NO: 8 - protein, 4 aa, synthetic construct
GGGS

SEQ ID NO: 9 - protein, 10 aa, synthetic construct
LRELHLNNNC

SEQ ID NO: 10 - protein, 10 aa, synthetic construct
LRELHLDNNC

SEQ ID NO: 11 - protein, 344 aa, synthetic construct
CGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 12 - protein, 222 aa, synthetic construct
CQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLC

SEQ ID NO: 13 - protein, 196 aa, synthetic construct
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICTG

SEQ ID NO: 14 - protein, 193 aa, synthetic construct
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRI

SEQ ID NO: 15 - protein, 193 aa, synthetic construct
CSQPLDVVLLLDGSSSLPESSFDKMKSFAKAFISKANIGPHLTQVSVIQYGSINTIDVPWNVVQEKAHLQSLVDLMQQEGGPSQIGDALAFAVRYVTSQIHGARPGASKAVVIIIMDTSLDPVDTAADAARSNRVAVFPVGVGDRYDEAQLRILAGPGASSNVVKLQQVEDLSTMATLGNSFFHKLCSGFSGV

SEQ ID NO: 16 - protein, 2813 aa, synthetic construct
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 17 - protein, 205 aa, synthetic construct
CSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSG

SEQ ID NO: 18 - protein, 170 aa, synthetic construct
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 19 - protein, 30 aa, synthetic construct
RRRPKGRGKRRREKQRPTDCHLCGDAVPRR

SEQ ID NO: 20 - protein, 22 aa, synthetic construct
RRRPKGRGKRRREKQRPTDCHL

SEQ ID NO: 21 - protein, 18 aa, synthetic construct
RRPKGRGKRRREKQRPTD

SEQ ID NO: 22 - protein, 15 aa, synthetic construct
RRRPKGRGKRRREKQ

SEQ ID NO: 23 - protein, 8 aa, synthetic construct
GKRRREKQ

SEQ ID NO: 24 - protein, 8 aa, synthetic construct
RRRPKGRG

SEQ ID NO: 25 - protein, 21 aa, synthetic construct
RRKTKGKRKRSRNSQTEEPHP

SEQ ID NO: 26 - protein, 98 aa, synthetic construct
KPVSLSYRCPCRFFESHVARANVKHLKILNTPNCALQIVARLKNNNRQVCIDPKLKWIQEYLEKALNKGRREEKVGKKEKIGKKKRQKKRKAAQKRKN

SEQ ID NO: 27 - protein, 30 aa, synthetic construct
GRREEKVGKKEKIGKKKRQKKRKAAQKRKN

SEQ ID NO: 28 - protein, 402 aa, synthetic construct
PIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 29 - protein, 1007 aa, synthetic construct
PIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSGGGSGGGSGGGSDAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 30 - protein, 1017 aa, synthetic construct
PRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSGGGSGGGSGGGSDAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 31 - protein, 634 aa, synthetic construct
PIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASEPKSCDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 32 - protein, 644 aa, synthetic construct
PRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASEPKSCDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 33 - protein, 422 aa, synthetic construct
RAGPPAALTLPRVQSRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 34 - protein, 422 aa, synthetic construct
RAGPPAALTLPRVQSRASRYPIAVDSSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 35 - protein, 422 aa, synthetic construct
RAGPPAALTLPRVQSRASRYPIAVDSSWTLPPAPNSTSPVSFIATYRLGMAARGHSWPSLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 36 - protein, 422 aa, synthetic construct
RAGPPAALTLPRVQSRASRYPIAVDSSWTLPPAPNSTSPVSFIATYRLGMAARGHSWPSLQQTPTSTSSTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 37 - protein, 1027 aa, synthetic construct
RAGPPAALTLPRVQSRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSGGGSGGGSGGGSDAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 38 - protein, 1027 aa, synthetic construct
RAGPPAALTLPRVQSRASRYPIAVDSSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSGGGSGGGSGGGSDAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 39 - protein, 1027 aa, synthetic construct
RAGPPAALTLPRVQSRASRYPIAVDSSWTLPPAPNSTSPVSFIATYRLGMAARGHSWPSLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSGGGSGGGSGGGSDAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 40 - protein, 1027 aa, synthetic construct
RAGPPAALTLPRVQSRASRYPIAVDSSWTLPPAPNSTSPVSFIATYRLGMAARGHSWPSLQQTPTSTSSTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSGGGSGGGSGGGSDAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 41 - protein, 654 aa, synthetic construct
RAGPPAALTLPRVQSRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASEPKSCDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 42 - protein, 654 aa, synthetic construct
RAGPPAALTLPRVQSRASRYPIAVDSSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASEPKSCDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 43 - protein, 654 aa, synthetic construct
RAGPPAALTLPRVQSRASRYPIAVDSSWTLPPAPNSTSPVSFIATYRLGMAARGHSWPSLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASEPKSCDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 44 - protein, 654 aa, synthetic construct
RAGPPAALTLPRVQSRASRYPIAVDSSWTLPPAPNSTSPVSFIATYRLGMAARGHSWPSLQQTPTSTSSTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASEPKSCDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 45 - protein, 634 aa, synthetic construct
PIAVDSSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASEPKSCDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 46 - protein, 634 aa, synthetic construct
PIAVDSSWTLPPAPNSTSPVSFIATYRLGMAARGHSWPSLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASEPKSCDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 47 - protein, 634 aa, synthetic construct
PIAVDSSWTLPPAPNSTSPVSFIATYRLGMAARGHSWPSLQQTPTSTSSTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASEPKSCDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 48 - protein, 402 aa, synthetic construct
PIAVDCSWTLPPAPNSTSPVSFIATYRLGMAARGHSWPSLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 49 - protein, 402 aa, synthetic construct
PIAVDCSWTLPPAPNSTSPVSFIATYRLGMAARGHSWPSLQQTPTSTSSTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 50 - protein, 402 aa, synthetic construct
PIAVDSSWTLPPAPNSTSPVSFIATYRLGMAARGHSWPSLQQTPTSTSSTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 51 - protein, 1007 aa, synthetic construct
PIAVDCSWTLPPAPNSTSPVSFIATYRLGMAARGHSWPSLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSGGGSGGGSGGGSDAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 52 - protein, 1007 aa, synthetic construct
PIAVDSSWTLPPAPNSTSPVSFIATYRLGMAARGHSWPSLQQTPTSTSSTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSGGGSGGGSGGGSDAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 53 - protein, 1222 aa, synthetic construct
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIGSRAGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSGGGSGGGSGGGSDAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 54 - protein, 617 aa, synthetic construct
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIGSRAGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 55 - protein, 617 aa, synthetic construct
RAGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIGS

SEQ ID NO: 56 - protein, 1026 aa, synthetic construct
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSGGGSGGGSGGGSGGGSRAGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNA

SEQ ID NO: 57 - protein, 1026 aa, synthetic construct
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAGGGSGGGSGGGSGGGSRAGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 58 - protein, 421 aa, synthetic construct
RNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAGGGSGGGSGGGSGGGSRAGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 59 - protein, 653 aa, synthetic construct
RNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAGGGSGGGSGGGSGGGSRAGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKEPKSCDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 60 - protein, 443 aa, synthetic construct
RAGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKRNLPVATPDPGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASDICLPRWGCLW

SEQ ID NO: 61 - protein, 423 aa, synthetic construct
PIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKRNLPVATPDPGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASDICLPRWGCLW

SEQ ID NO: 62 - protein, 423 aa, synthetic construct
PIAVDCSWTLPPAPNSTSPVSFIATYRLGMAARGHSWPSLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKRNLPVATPDPGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASDICLPRWGCLW

SEQ ID NO: 63 - protein, 423 aa, synthetic construct
PIAVDSSWTLPPAPNSTSPVSFIATYRLGMAARGHSWPSLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKRNLPVATPDPGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASDICLPRWGCLW

SEQ ID NO: 64 - protein, 423 aa, synthetic construct
PIAVDSSWTLPPAPNSTSPVSFIATYRLGMAARGHSWPSLQQTPTSTSSTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKRNLPVATPDPGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASDICLPRWGCLW

SEQ ID NO: 65 - protein, 1011 aa, synthetic construct
RAGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGDAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 66 - protein, 991 aa, synthetic construct
PIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGDAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 67 - protein, 991 aa, synthetic construct
PIAVDCSWTLPPAPNSTSPVSFIATYRLGMAARGHSWPSLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGDAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 68 - protein, 991 aa, synthetic construct
PIAVDSSWTLPPAPNSTSPVSFIATYRLGMAARGHSWPSLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGDAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 69 - protein, 991 aa, synthetic construct
PIAVDSSWTLPPAPNSTSPVSFIATYRLGMAARGHSWPSLQQTPTSTSSTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGDAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 70 - protein, 657 aa, synthetic construct
RAGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPAARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 637 aa, synthetic construct
PIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPAARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 637 aa, synthetic construct
PIAVDCSWTLPPAPNSTSPVSFIATYRLGMAARGHSWPSLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPAARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 637 aa, synthetic construct
PIAVDSSWTLPPAPNSTSPVSFIATYRLGMAARGHSWPSLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPAARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 637 aa, synthetic construct
PIAVDSSWTLPPAPNSTSPVSFIATYRLGMAARGHSWPSLQQTPTSTSSTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPAARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 4 aa, synthetic construct
GGGG

SEQ ID NO: 76 - protein, 8 aa, synthetic construct
GGGGGGGG

SEQ ID NO: 77 - protein, 5 aa, synthetic construct
GGGGS

SEQ ID NO: 78 - protein, 10 aa, synthetic construct
GGGGSGGGGS

SEQ ID NO: 79 - protein, 15 aa, synthetic construct
GGGGSGGGGSGGGGS

SEQ ID NO: 80 - protein, 5 aa, synthetic construct
PAPAP

SEQ ID NO: 81 - protein, 5 aa, synthetic construct
EAAAK

SEQ ID NO: 82 - protein, 10 aa, synthetic construct
EAAAKEAAAK

SEQ ID NO: 83 - protein, 15 aa, synthetic construct
EAAAKEAAAKEAAAK

SEQ ID NO: 84 - protein, 17 aa, synthetic construct
AEAAAKEAAAKEAAAKA

SEQ ID NO: 85 - protein, 26 aa, synthetic construct
AEAAAKEAAAKALEAEAAAKEAAAKA

SEQ ID NO: 86 - protein, 232 aa, synthetic construct
EPKSCDKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 87 - protein, 227 aa, synthetic construct
DKTHTCPPCPAPEFEGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 195 aa, synthetic construct
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIGS

SEQ ID NO: 89 - protein, 21 aa, synthetic construct
METDTLLLWVLLLWVPGSTGD

SEQ ID NO: 90 - protein, 209 aa, synthetic construct
RKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 91 - protein, 1027 aa, synthetic construct
RKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSGGGSGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 92 - protein, 189 aa, synthetic construct
PIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 93 - protein, 11 aa, synthetic construct
DICLPRWGCLW